SEQUENCE LISTING (43 sequences)

SEQ ID NO: 1 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRYLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 2 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 3 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRYLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 4 - protein, 579 aa, Unknown
  Description of Unknown Neurospora crassa sequence
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 5 - protein, 918 aa, Saccharomyces cerevisiae [sequence omitted]

SEQ ID NO: 6 - protein, 884 aa, Saccharomyces cerevisiae
MDPNSNSSSETLRQEKQGFLDKALQRVKGIALRRNNSNKDHTTDDTTGSIRTPTSLQRQNSDRQSNMTSVFTDDISTIDDNSILFSEPPQKQSMMMSICVGVFVAVGGFLFGYDTGLINSITSMNYVKSHVAPNHDSFTAQQMSILVSFLSLGTFFGALTAPFISDSYGRKPTIIFSTIFIFSIGNSLQVGAGGITLLIVGRVISGIGIGAISAVVPLYQAEATHKSLRGAIISTYQWAITWGLLVSSAVSQGTHARNDASSYRIPIGLQYVWSSFLAIGMFFLPESPRYYVLKDKLDEAAKSLSFLRGVPVHDSGLLEELVEIKATYDYEASFGSSNFIDCFISSKSRPKQTLRMFTGIALQAFQQFSGINFIFYYGVNFFNKTGVSNSYLVSFITYAVNVVFNVPGLFFVEFFGRRKVLVVGGVIMTIANFIVAIVGCSLKTVAAAKVMIAFICLFIAAFSATWGGVVWVISAELYPLGVRSKCTAICAAANWLVNFICALITPYIVDTGSHTSSLGAKIFFIWGSLNAMGVIVVYLTVYETKGLTLEEIDELYIKSSTGVVSPKFNKDIRERALKFQYDPLQRLEDGKNTFVAKRNNFDDETPRNDFRNTISGEIDHSPNQKEVHSIPERVDIPTSTEILESPNKSSGMTVPVSPSLQDVPIPQTTEPAEIRTKYVDLGNGLGLNTYNRGPPSLSSDSSEDYTEDEIGGPSSQGDQSNRSTMNDINDYMARLIHSTSTASNTTDKFSGNQSTLRYHTASSHSDTTEEDSNLMDLGNGLALNAYNRGPPSILMNSSDEEANGGETSDNLNTAQDLAGMKERMAQFAQSYIDKRGGLEPETQSNILSTSLSVMADTNEHNNEILHSSEENATNQPVNENNDLK

SEQ ID NO: 7 - protein, 763 aa, Saccharomyces cerevisiae
MNDSQNCLRQREENSHLNPGNDFGHHQGAECTINHNNMPHRNAYTESTNDTEAKSIVMCDDPNAYQISYTNNEPAGDGAIETTSILLSQPLPLRSNVMSVLVGIFVAVGGFLFGYDTGLINSITDMPYVKTYIAPNHSYFTTSQIAILVSFLSLGTFFGALIAPYISDSYGRKPTIMFSTAVIFSIGNSLQVASGGLVLLIVGRVISGIGIGIISAVVPLYQAEAAQKNLRGAIISSYQWAITIGLLVSSAVSQGTHSKNGPSSYRIPIGLQYVWSSILAVGMIFLPESPRYYVLKDELNKAAKSLSFLRGLPIEDPRLLEELVEIKATYDYEASFGPSTLLDCFKTSENRPKQILRIFTGIAIQAFQQASGINFIFYYGVNFFNNTGVDNSYLVSFISYAVNVAFSIPGMYLVDRIGRRPVLLAGGVIMAIANLVIAIVGVSEGKTVVASKIMIAFICLFIAAFSATWGGVVWVVSAELYPLGVRSKCTAICAAANWLVNFTCALITPYIVDVGSHTSSMGPKIFFIWGGLNVVAVIVVYFAVYETRGLTLEEIDELFRKAPNSVISSKWNKKIRKRCLAFPISQQIEMKTNIKNAGKLDNNNSPIVQDDSHNIIDVDGFLENQIQSNDHMIAADKGSGSLVNIIDTAPLTSTEFKPVEHPPVNYVDLGNGLGLNTYNRGPPSIISDSTDEFYEENDSSYYNNNTERNGANSVNTYMAQLINSSSTTSNDTSFSPSHNSNARTSSNWTSDLASKHSQYTSPQ

SEQ ID NO: 8 - protein, 961 aa, Saccharomyces cerevisiae
MITEGFPPNLNALKGSSLLEKRVDSLRQLNTTTVNQLLGLPGMTSTFTAPQLLQLRIIAITASAVSLIAGCLGMFFLSKMDKRRKVFRHDLIAFLIICDFLKAFILMIYPMIILINNSVYATPAFFNTLGWFTAFAIEGADMAIMIFAIHFAILIFKPNWKWRNKRSGNMEGGLYKKRSYIWPITALVPAILASLAFINYNKLNDDSDTTIILDNNNYNFPDSPRQGGYKPWSAWCYLPPKPYWYKIVLSWGPRYFIIIFIFAVYLSIYIFITSESKRIKAQIGDFNHNVLEEEKEKKKLFGLGHWGKAKWYFRSYFKLPLLHLLRNLKNFFTISFIDPNEETDDSGSSNGTFNFGESSNEIPTLFRKTNTGSDENVSASGGVRLLDYNSAKPLDMSKYAMSEQPDLERNNPFDCENDITLNPSELVSKQKEHKVTFSVENEGLDTRKSSMLGHQTFSCQNSLESPLAMYDNKNDNSDITSNIKEKGGIINNNSNNDDDDNNNNNDNDNDNNNSNNNNNNNNNNNNNNNNNNNNNNNNNNNNNNSNNIKNNVDNNNTNPADNIPTLSNEAFTPSQQFSQERVNNNADRCENSSFTNVQQHFQAQTYKQMKKRRAQIQKNLRAIFIYPLSYIGIWLFPIIADALQYNHEIKHGPTMWVTYIDTCVRPLSCLVDVIVYLFKEKPWNYSWAKTESKYLIEKYILKGELGEKEILKFCHSNWGKRGWYYRGKWKKRKCWKYSTNPLKRILWFVERFFKQLFELKLHFSFYDNCDDFEYWENYYSAKDSNDNKRTESDETKTNSSDRSLPSNSLELQAMLNNITAEEVEVPLFWRIIHHIPMLGGIDLDELNRLLKIRYNNDHFSLPGLKFALNQNKSHDKHQDVSTNSMVKSSFFSSNIVTNDDENSIEEDKNLRYSDASASENYLVKPTIPGTTPDPIIEAQNDNDSSDSSGIDLIAFLRNGPL

SEQ ID NO: 9 - protein, 525 aa, Unknown
  Description of Unknown Neurospora crassa sequence
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA

SEQ ID NO: 10 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDASLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 11 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWLGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 12 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFVPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 13 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIAGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 14 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLAATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 15 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRWLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 16 - DNA, 1740 bp, Artificial Sequence
  Description of Artificial Sequence Synthetic polynucleotide
atgtcctctcatggttctcatgatggtgcttctactgaaaaacatttggccactcatgatattgctccaactcatgatgctatcaagatcgtgcccaagggccatggccagacagccacaaagcccggcgcccaagagaaggaggtccgcaacgccgccctatttgcggccatcaaggagtccaatatcaagccctggagcaaggagtccatccacctctatttcgccatcttcgtcgccttttgttgtgcatgcgccaacggttacgatggttcactcatgaccggaatcatcgctatggacaagttccagaaccaattccacactggtgacactggtcctaaagtctctgtcatcttttctctctataccgtgggtgccatggttggagctcccttcgctgctatcctctctgatcgttttggccgtaagaagggcatgttcatcggtggtatctttatcattgtcggctccattattgttgctagctcctccaagctcgctcagtttgtcgttggccgcttcgttcttggcctcggtatcgccatcatgaccgttgctgccccggcctactccatcgaaatcgcccctcctcactggcgcggccgctgcactggcttctacagctgcggttggttcggaggttcgattcctgccgcctgcatcacctatggctgctacttcattaagagcaactggtcatggcgtatccccttgatccttcaggctttcacgtgccttatcgtcatgtcctccgtcttcttcctcccagaatcccctcgctacctatttgccaacggccgcgacgctgaggctgttgcctttcttgtcaagtatcacggcaacggcgatcccaattccaagctggtgttgctcgagactgaggagatgagggacggtatcaggaccgacggtgtcgacaaggtctggtgggattaccgcccgctcttcatgacccacagcggccgctggcgcatggcccaggtgctcatgatctccatctttggccagttctccggcaacggtctcggttacttcaataccgtcatcttcaagaacattggtgtcaccagcacctcccaacagctcgcctacaacatcctcaactccgtcatctccgctatcggtgccttgaccgccgtctccatgactgatcgtatgccccgccgcgcggtgctcattatcggtaccttcatgtgcgccgctgctcttgccaccaactcgggtctttcggctactctcgacaagcagactcaaagaggcacgcaaatcaacctgaaccagggtatgaacgagcaggatgccaaggacaacgcctacctccacgtcgacagcaactacgccaagggtgccctggccgcttacttcctcttcaacgtcatcttctccttcacctacactcccctccagggtgttattcccaccgaggctctcgagaccaccatccgtggcaagggtcttgccctttccggcttcattgtcaacgccatgggcttcatcaaccagttcgctggccccatcgctctccacaacattggctacaagtacatctttgtctttgtcggctgggatcttatcgagaccgtcgcttggtacttctttggtgtcgaatcccaaggccgtaccctcgagcagctcgaatgggtctacgaccagcccaaccccgtcaaggcctccctaaaagtcgaaaaggtcgtcgtccaggccgacggccatgtgtccgaagctatcgttgcttaa

SEQ ID NO: 17 - protein, 349 aa, Neisseria meningitidis
MPSEAFRRHRAYRENKLQPLVSVLICAYNVEKYFAQSLAAVVNQTWRNLDILIVDDGSTDGTLAIAQRFQEQDGRIRILAQPRNSGLIPSLNIGLDELAKSGGGGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 18 - protein, 339 aa, Neisseria polysaccharea
MICAYNVEKYFAQALDAVVGQTWRNLDILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAFFPFGNPIHNNTMIMRRSVIDGGLRYNTERDWAEDYQFWYDVSKLGRLAYYPEALVKYRLHANQVSSKHSIRQHEIAQGIQKTARNDFLQSMGFKTRFDSLEYRQTKAAAYELLEKDLPEEDFERARRFLYQCFKRTDTPPAGAWLDFAADGRMRRLFTLRQYFSILHRLIKNRRQARSDSAGKEQEI

SEQ ID NO: 19 - protein, 395 aa, Homo sapiens
MSVGRRRIKLLGILMMANVFIYFIMEVSKSSSQEKNGKGEVIIPKEKFWKISTPPEAYWNREQEKLNRQYNPILSMLTNQTGEAGRLSNISHLNYCEPDLRVTSVVTGFNNLPDRFKDFLLYLRCRNYSLLIDQPDKCAKKPFLLLAIKSLTPHFARRQAIRESWGQESNAGNQTVVRVFLLGQTPPEDNHPDLSDMLKFESEKHQDILMWNYRDTFFNLSLKEVLFLRWVSTSCPDTEFVFKGDDDVFVNTHHILNYLNSLSKTKAKDLFIGDVIHNAGPHRDKKLKYYIPEVVYSGLYPPYAGGGGFLYSGHLALRLYHITDQVHLYPIDDVYTGMCLQKLGLVPEKHKGFRTFDIEEKNKNNICSYVDLMLVHSRKPQEMIDIWSQLQSAHL

SEQ ID NO: 20 - protein, 265 aa, Escherichia coli
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 21 - protein, 326 aa, Homo sapiens
MASKVSCLYVLTVVCWASALWYLSITRPTSSYTGSKPFSHLTVARKNFTFGNIRTRPINPHSFEFLINEPNKCEKNIPFLVILISTTHKEFDARQAIRETWGDENNFKGIKIATLFLLGKNADPVLNQMVEQESQIFHDIIVEDFIDSYHNLTLKTLMGMRWVATFCSKAKYVMKTDSDIFVNMDNLIYKLLKPSTKPRRRYFTGYVINGGPIRDVRSKWYMPRDLYPDSNYPPFCSGTGYIFSADVAELIYKTSLHTRLLHLEDVYVGLCLRKLGIHPFQNSGFNHWKMAYSLCRYRRVITVHQISPEEMHRIWNDMSSKKHLRC

SEQ ID NO: 22 - protein, 247 aa, Salmonella enterica
MLPVNKFNPYLDRAIHSILSQSYPSIELIIIANNCTNDFFDALKKRECETIKVLRTNIAYLPYCLNKGLDLCNGDFVARMDSDDISHPERIDRQVDFLINNPDIDVVGTNAVYIDEDDVELEKSNLPENNNAIKKMLPYKCCLVHPSVMFRKNVVISSGGYMFANYSEDYELWNRLAVEGRTFYNLSEYLLYYRLHNNQSTSKNNLFMVMVNDVAIKVKYFLLTKKVSYLLGIIRTVFSVFYCKYIK

SEQ ID NO: 23 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 24 - protein, 279 aa, Neisseria polysaccharea
MQNHVISLSSAAERRAHIAATFGAHGIPFQFFDALMPSERLEQAMAELVLGLSAHPYLSGVEKACFMSHAVLWKQALDEGTPYIAVFEDDVLLSEGAEQFLAEDAWLQERFDPDSAFIVRLETMFMHVLTSPSGVADYCGRAFPLLESEHWGTAGYIISKKAMRFFLDRFAALPSEGLHPIDWMMFGNPDDRERMPVFQLNPALCAQELHYAKFHDQNSALGSLIEHDRCLNRKQQRRDSPANTFKHRLIRALTKISREREKRRQRREQLIGKIIVPFQ

SEQ ID NO: 25 - protein, 382 aa, Homo sapiens
MSVLRRMMRVSNRSLLAFIFFFSLSSSCLYFIYVAPGIANTYLFMVQARGIMLRENVKTIGHMIRLYTNKNSTLNGTDYPEGNNSSDYLVQTTTYLPENFTYSPYLPCPEKLPYMRGFLNVNVSEVSFDEIHQLFSKDLDIEPGGHWRPKDCKPRWKVAVLIPFRNRHEHLPIFFLHLIPMLQKQRLEFAFYVIEQTGTQPFNRAMLFNVGFKEAMKDSVWDCVIFHDVDHLPENDRNYYGCGEMPRHFAAKLDKYMYILPYKEFFGGVSGLTVEQFRKINGFPNAFWGWGGEDDDLWNRVHYAGYNVTRPEGDLGKYKSIPHHHRGEVQFLGRYKLLRYSKERQYIDGLNNLIYRPKILVDRLYTNISVNLMPELAPIEDY

SEQ ID NO: 26 - protein, 343 aa, Campylobacter jejuni
MQIKIDKLTISQKNPLIIPEIGINHNGSLEIAKLMVDAAKRAGAKIIKHQTHIVEDEMSQEAKNVIPGNANISIYEIMEQCALNYKDELALKEYVEKQGLVYLSTPFSRAAANRLEDMGVSAYKIGSGECNNYPLIKHIAQFKKPMIISTGMNSIESIKPTVKILRDYEIPFVLLHTTNLYPTPSHLVRLQAMLELYKEFNCLYGLSDHTTNNLACIGAIALGASVLERHFTDTMDRKGPDIVCSMDESTLKDLINQTQEMVLLRGDNNKNPLKEEQVTIDFAFASVVSIKDIKKGEILSMDNIWVKRPSKGGISAKDFEAILGKRAKKDIKNNIQLTWDDFE

SEQ ID NO: 27 - protein, 346 aa, Escherichia coli
MSNIYIVAEIGCNHNGSVDIAREMILKAKEAGVNAVKFQTFKADKLISAIAPKAEYQIKNTGELESQLEMTKKLEMKNHYYLHLMEYAVSLNLDVFSTPFDEDSIDFLASLKQKIWKIPSGELLNLPYLEKIAKLPIPDKKIIISTGMATIDEIKQSVSIFINNKVPVGNITILHCNTEYPTPFEDVNLNAINDLKKHFPKNNIGFSDHSSGFYAAIAAVPYGITFIEKHFTLDKSMSGPDHLASIEPDELKHLCIGVRCVEKSLGSNSKVVTASERKNKIVARKSIIAKTEIKKGEVFSEKNITTKRPGNGISPMEWYNLLGKIAEQDFIPDELIIHSEFKNQGE

SEQ ID NO: 28 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 29 - protein, 228 aa, Neisseria meningitidis
MEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 30 - protein, 221 aa, Campylobacter jejuni
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHIDKAFNLYKNSNANALISVSECDNKILKAFVCNDYGDLAGICNDEYPFMPRQKLPKTYMSNGAIYILKIKEFLNNPSFLQNKTKHFLMDESSSLDIDCLEDLKKVEQIWKK

SEQ ID NO: 31 - protein, 382 aa, Neisseria meningitidis
MFNLSEWSFRDMGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 32 - protein, 642 aa, Unknown
  Description of Unknown Trypanosoma cruzi sequence
MLAPGSSRVELFKRQSSKVPFEKDGKVTERVVHSFRLPALVNVDGVMVAIADARYETSNDNSLIDTVVKYSVDDGETWETQIAIKNSRASSVSRVVDPTVIVKGNKLYVLVGSYNSSRSYWTSHGDARDWNILLAVGEVTKSTAGGKITASIKWGSPVSLKEFFPAEMEGMHTNQFLGGAGVAIVASNGNLVYPVQVTNKRKQVFSKIFYSEDDGKTWKFGKGRSDFGCSEPVALEWEGKLIINTRVDYRRRLVYESSDMGNSWVEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPADPAASSSERGCGPAVTTVGLVGFLSHSATKTEWEDAYRCVNASMANAERVPNGLKFAGVGGGALWPVSQQGQNQRYRFANHAFTVVASVTIHEVPSVASPLLGASLDSSGGKKLLGLSYDEKHQWQPIYGSTPVTPTGSWETGKRYHVVLTMANKIGSVYIDGEPLEGSGQTVVPDERTPDISHFYVGGYKRSDMPTISHVTVNNVLLYNRQLNAEEIRTLFLSQDLIGTEAHMDSSSDTSA

SEQ ID NO: 33 - protein, 371 aa, Neisseria gonorrhoeae
MGLKKVCLTVLCLIVFCFGIFYTFDRVNQGERNAVSLLKDKLFNEEGKPVNLIFCYTILQMKVAERIMAQHPGERFYVVLMSENRNEKYDYYFNQIKDKAERAYFFYLPYGLNKSFNFIPTMAELKVKSMLLPKVKRIYLASLEKVSIAAFLSTYPDAEIKTFDDGTNNLIRESSYLGGEFAVNGAIKRNFARMMVGDWSIAKTRNASDEHYTIFKGLKNIMDDGRRKMTYLPLFDASELKAGDETGGTVRILLGSPDKEMKEISEKAAKNFNIQYVAPHPRQTYGLSGVTALNSPYVIEDYILREIKKNPHTRYEIYTFFSGAALTMKDFPNVHVYALKPASLPEDYWLKPVYALFRQADIPILTFDDKN

SEQ ID NO: 34 - protein, 514 aa, Photobacterium sp.
MKNFLLLTLILLTACNNSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 35 - protein, 412 aa, Pasteurella multocida
MKNRRLNFKLFFLIIFSLFSTLSWSKTITLYLDPASLPALNQLMDFTQNNEDKTHPRIFGLSRFKIPDNIITQYQNIHFVELKDNRPTEALFTILDQYPGNIELNIHLNIAHSVQLIRPILAYRFKHLDRVSIQQLNLYDDGSMEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAEGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 36 - protein, 391 aa, Synechocystis sp.
MIAHRRQELAQQYYQALHQDVLPFWEKYSLDRQGGGYFTCLDRKGQVFDTDKFIWLQNRQVWQFAVFYNRLEPKPQWLEIARHGADFLARHGRDQDGNWYFALDQEGKPLRQPYNVFSDCFAAMAFSQYALASGAQEAKAIALQAYNNVLRRQHNPKGQYEKSYPGTRPLKSLAVPMILANLTLEMEWLLPPTTVEEVLAQTVREVMTDFLDPEIGLMREAVTPTGEFVDSFEGRLLNPGHGIEAMWFMMDIAQRSGDRQLQEQAIAVVLNTLEYAWDEEFGGIFYFLDRQGHPPQQLEWDQKLWWVHLETLVALAKGHQATGQEKCWQWFERVHDYAWSHFADPEYGEWFGYLNRRGEVLLNLKGGKWKGCFHVPRALWLCAETLQLPVS

SEQ ID NO: 37 - protein, 391 aa, Escherichia coli
MKKILYVTGSRAEYGIVRRLLTMLRETPEIQLDLAVTGMHCDNAYGNTIHIIEQDNFNIIKVVDININTTSHTHILHSMSVCLNSFGDFFSNNTYDAVMVLGDRYEIFSVAIAASMHNIPLIHIHGGEKTLANYDEFIRHSITKMSKLHLTSTEEYKKRVIQLGEKPGSVFNIGSLGAENALSLHLPNKQELELKYGSLLKRYFVVVFHPETLSTQSVNDQIDELLSAISFFKNTHDFIFIGSNADTGSDIIQRKVKYFCKEYKFRYLISIRSEDYLAMIKYSCGLIGNSSSGLIEVPSLKVATINIGDRQKGRVRGASVIDVPVEKNAIVRGINISQDEKFISVVQSSSNPYFKENALINAVRIIKDFIKSKNKDYKDFYDIPECTTSYD

SEQ ID NO: 38 - protein, 378 aa, Acinetobacter baumannii
MKKIAVFTGTRAEYGLLYWLMKDIQQDPELELQILATAMHYSPEHGETWKTIVQDGFEITESVEMLLSSDTSSAVVKSMGVGLLGFADALKRMQPNLLVVLGDRFEALAVTQAALIMQVPVAHLHGGEITEGAYDESIRHAITKMSNIHFAAAEEYKKRIIQLGEQPEKVFNVGALGLDHIQRTTFKNIAELSELYDFDFSKPYFLITYHPETNLLEENVSPLFDALKQIKDVNFVFSYPNADNGNTNIVKAMLDLKAQLPDRVLLVKSFGIQNYLSVLKNALAMVGNSSSGLSEAPALQVPTVNIGDRQKGRLRCESILDVKLDENEILEALQKAINFPDDQLSQVVPPLGLGNTSQKIIELIKTTDFRKKAPFYDL

SEQ ID NO: 39 - protein, 377 aa, Neisseria meningitidis
MKRILCITGTRADFGKLKPLLAYIENHPDLELHLIVTGMHMMKTYGRTYKEVTRENYQHTYLFSNQIQGEPMGAVLGNTITFISRLSDEIEPDMVMIHGDRLEALAGAAVGALSSRLVCHIEGGELSGTVDDSIRHSISKLSHIHLVANEQAVTRLVQMGEKRKHIHIIGSPDLDVMASSTLPSLEEVKEYYGLPYENYGISMFHPVTTEAHLMPQYAAQYFKALELSGQNIISIYPNNDTGTESILQELLKYQSDKFIAFPSIRFEYFLVLLKHAKFMVGNSSAGIREAPLYGVPSIDVGTRQNNRHMGKSIIHTDYETKNIFDAIQQACSLGKFEADDTFNGGDTRTSTERFAEVINNPETWNVSAQKRFIDLNL

SEQ ID NO: 40 - protein, 384 aa, Streptococcus agalactiae
MKKICLVTGSRAEYGIMKPLIQRLSKDKEVNLQIIATAMHLEEKYGYTYRQIEEDGFDIAYKVPLHLYDTDRRTVSTAMAHLQLGLTKIFDKEDYDLVIILGDRYEMLPVVNVALIYNVPVCHLHGGETSLGNFDEYIRHAITKMSHLHLVSTEDFRQRVIQMGEQPQFVINTGALGVENALSIPSLTKEAIEKQLGIVLEESYFVVLYHPVTFEQGKSAGEQMKAVLSALSKFGVQCLFIGSNSDTGSDDIAKAINTYLINHENSYCFASLSTQLYHSLIRHSLGLIGNSSSGLIEVPSLMKPTLNIGDRQKGRLHGESVVSVPVETPSVLEGLSKLNEVTNFDNPYYKENASSIAYEAIKLYLKDEPSISQPFYDLKENNLK

SEQ ID NO: 41 - protein, 587 aa, Kluyveromyces sp.
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 42 - protein, 333 aa, Neisseria meningitidis
MQPLVSVLICAYNVEKYFAQSLAAVVNQTWRNLDILIVDDGSTDGTLAIAQRFQEQDGRIRILAQPRNSGLIPSLNIGLDELAKSGGGGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 43 - protein, 5 aa, Unknown
  Description of Unknown PESPR motif sequence
PESPR

(19) United States
(12) Patent Application Publication
(10) Pub. No.: US 2023/0183767 A1
(43) Pub. Date: Jun. 15, 2023

(54) METHODS FOR PRODUCTION OF OLIGOSACCHARIDES

(71) Applicant: Zimitech, Inc., Alameda, CA (US)

(72) Inventors: Jingjing Liu, Moraga, CA (US); James Harrison Doudna Cate, Berkeley, CA (US)

(21) Appl. No.: 17/916,695

(22) PCT Filed: Apr. 1, 2021
(86) PCT No.: PCT/US21/25394

§ 371 Date: Oct. 3, 2022

Related U.S. Application Data
(60) Provisional application No. 63/003,590, filed on Apr. 1, 2020.

(51) Int. Cl. C12P 19/18; C12N 9/10; C12N 9/12; C12N 9/90; A23L 33/135; A23L 33/125; A23L 33/00; A23K 10/18; A23K 20/163

(52) U.S. Cl. CPC C12P 19/18; C12N 9/1051; C12N 9/1085; C12Y 205/01056; C12N 9/1081; C12Y 204/99001; C12Y 204/99004; C12N 9/1241; C12Y 207/07043; C12N 9/90; C12Y 501/03008; A23L 33/135; A23L 33/125; A23L 33/40; A23K 10/18; A23K 20/163

(57) ABSTRACT

Disclosed herein are genetically modified microorganisms and related methods for the enhanced production and export of oligosaccharides. The microorganisms described herein express major facility superfamily proteins such as CDT-1, which allows for the export of oligosaccharides. Variants of CDT-1 exhibit higher activity regarding oligosaccharide export. The microorganisms described herein express formation enzymes for the production of oligosaccharides. Means to export oligosaccharides into the growth medium are provided herein.

### RELATED APPLICATIONS

This application claims the benefit of priority to U.S. Patent Application Ser. No. 63/003,590, filed Apr. 1, 2020, which is incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 30, 2021, is named ZTW-00425_SL.txt and is 171,287 bytes in size.

### BACKGROUND

Functional oligosaccharides have emerged as valuable components of food and dietary supplements. Their resistance to digestion and fermentation by colonic microbes has given oligosaccharides a nutritional edge. Apart from implications as dietary fibers, sweeteners, and humectants, they are hailed as prebiotics. Their beneficial effects extend from anti-oxidant, anti-inflammatory, immunomodulatory, anti-hypertensive, and anti allergic to anti-cancer, neuroprotective, and improvement of the skin barrier function and hydration. The rising popularity of bioactive oligosaccharides has accelerated the search for their generation from new, sustainable sources.

Oligosaccharides may be obtained from natural sources and may also be synthesized. Various natural sources of oligosaccharides include milk, honey, sugarcane juice, rye, barley, wheat, soybean, lentils, mustard, fruits, and vegetables such as onion, asparagus, sugar beet, artichoke, chicory, leek, garlic, banana, yacon, tomato, and bamboo shoots. Common oligosaccharide manufacturing methods include hydrolysis of polysaccharides, chemical, and enzymatic polymerization from disaccharide or monosaccharide substrates. Acid, alkali, and enzymatic hydrolysis of polysaccharides can generate oligosaccharides of desired structure and functional properties. In certain cases, enzymatic methods are preferred for oligosaccharide synthesis due to their high selectivity and yields, and environmentally-friendly nature. In other cases, oligosaccharide-producing microbial strains may be engineered by introducing exogenous genes to enable oligosaccharide production.

### SUMMARY OF THE INVENTION

Oligosaccharides produced in microorganisms will accumulate intracellularly if not actively transported out of the cell into the medium from where they can be further isolated. Accumulation within the cells in the absence of export processes requires isolation of the oligosaccharide from biomass and limits conversion of the substrate to fermentation product or oligosaccharide. The lack of export of fermentation products out of cells also increases costs of the fermentation processes since fermentation runs effectively have to be stopped once the cells accumulate significant amounts of oligosaccharide in order to recover the latter. In addition, recovery of oligosaccharide from cells require additional processes such as extraction or breakage of cells, or both, which might additionally increase costs and require significant purification steps to remove contaminating cell debris, or both.

Exporter proteins for oligosaccharides are not readily available since organisms typically evolved mechanisms to import, not export, substrates for consumption, sensing or both. The identification of functional substrate transporters allowing for oligosaccharide export which is functional in eukaryotic cells is thus paramount for the production of oligosaccharides in yeasts and other eukaryotic production hosts.

It has been discovered that substrate importers might act as exporters. For example, if oligosaccharides accumulate to high concentrations within cells, this along with the appropriate transporter may drive substrate flow out of the cell where the concentration is lower. Additionally, mutagenized versions of transporters might be impaired in regulation of transport processes in such a way that substrate export along a concentration gradient is facilitated. Additionally, modification of the same substrate transporter can lead to higher fermentation product or oligosaccharide export rates if expressed in an organism accumulating a suitable substrate within the cell.

Accordingly, provided herein are transporters that can function as a substrate exporter, particularly for oligosaccharides. Such transporters can also function as importers, and import oligosaccharides, such as an oligosaccharide different from that exported.

CDT-1 (XP_963801.1) from the fungus Neurospora crassa is a substrate transporter from the major facilitator superfamily (MFS) that imports cellobiose into the cell. Unexpectedly, expression of a cellodextrin transporter in an engineered Saccharomyces cerevisiae strain capable of producing a lactose-based oligosaccharide, such as an Lacto-N-Triose II (LNTII)-derived HMO or a sialylated HMO, leads to an increase of an Lacto-N-Triose II (LNTII)-derived HMO or a sialylated HMO released into the culture medium. In such circumstances, CDT-1 acts as an exporter facilitating transport of oligosaccharides, such as a Lacto-N-Triose II (LNTII)-derived HMO or a sialylated HMO, out of the cell. Moreover, mutated versions of CDT-1 can act as Lacto-N-Triose II (LNTII)-derived HMO or sialylated HMO exporters and in some cases, such mutations further increase Lacto-N-Triose II (LNTII)-derived HMO or sialylated HMO export out of the cell, if compared to the non-mutated version of this transporter.

In certain aspects, the present disclosure provides Lacto-N-Triose II (LNTII)-derived HMO or sialylated HMO production strains expressing a transporter for export of the HMO from a cell of the production strain. In some embodiments, the transporter is a CDT such as CDT-1 or a or a variant of CDT1 (i.e., having one or more alterations in a CDT amino acid sequence).

In one aspect, an engineered microorganism capable of producing a human milk oligosaccharide (HMO) is provided. Numerous embodiments are further provided that can be applied to any aspect of the present invention described herein. For example, in some embodiments, the microorganism comprises a first heterologous gene encoding an HMO formation enzyme. In some embodiments, the microorganism further comprises a second heterologous gene encoding a transporter. In some embodiments, the transporter is CDT-1 or a variant thereof. In some embodiments, the HMO is a Lacto-N-Triose II (LNTII)-derived HMO or a sialylated HMO.

Compared to the parental microorganisms, the microorganisms described herein have an increased ability to produce oligosaccharide products of interest. Accordingly, methods of producing products of interest by culturing the microorganisms of the present disclosure in media containing the oligosaccharides and obtaining the products of interest from the media are provided.

In some embodiments, a CDT mutant is CDT-1SY. These strains show increased export of oligosaccharides if compared to their parental strains not expressing CDT-1 or a CDT-1 analogue.

In certain aspects, the present disclosure provides methods of producing oligosaccharides by culturing the microorganisms disclosed herein. In some embodiments, the microorganisms are bacteria or fungi, for example, filamentous fungi or yeasts. In some embodiments, the microorganisms are yeast, for example, Saccharomyces cerevisiae.

In one aspect a method of producing an oligosaccharide comprising culturing a microorganism described herein in a culture medium and recovering the oligosaccharide is provided herein. In another aspect, a method of isolating an HMO comprising: providing a culture medium with at least one carbon source; providing a microorganism described herein; and culturing the microorganism in the culture medium; wherein a substantial portion of the HMO is exported into the culture medium is provided. In another aspect, a method of isolating an HMO comprising: providing a culture medium with at least one carbon source; providing a microorganism capable of producing and exporting an HMO, wherein the microorganism comprises a heterologous transporter and one or more heterologous HMO production gene(s); and culturing the microorganism in the culture medium; wherein a substantial portion of the HMO is exported into the culture medium is provided.

In another aspect, a product suitable for animal consumption comprising the HMO produced by the microorganism described herein or according to the method described herein and at least one additional ingredient acceptable for animal consumption.

In another aspect, a product suitable for animal consumption comprising the microorganism described herein and optionally at least one additional ingredient acceptable for animal consumption.

In one aspect, provided herein is an engineered microorganism capable of producing a human milk oligosaccharide (HMO) comprising: a first heterologous gene encoding an HMO formation enzyme and a second heterologous gene encoding a variant of CDT-1, wherein the CDT-1 variant comprises a sequence having one or more amino acid replacements at positions corresponding to amino acid positions 91, 209, 213, 256, 262, 335, 411 of SEQ ID NO:4, or the CDT-1 variant is selected from the group consisting of CDT-1 N209S F262Y, CDT-1 G91A, CDT-1 F213L, CDT-1 L256V, CDT-1 F335A, CDT-1 S411A, and CDT-1 N209S F262W, or the CDT-1 variant comprises an amino acid replacement at a position near the sugar substrate binding pocket and/or the PESPR motif (SEQ ID NO: 43), such as 6336, Q337, N341, or G471; and wherein the engineered microorganism produces an HMO and is improved in the uptake of lactose into the microorganism as compared to a parent microorganism that lacks CDT-1, or variant thereof.

### BRIEF DESCRIPTION OF THE DRAWINGS

FIG. 1 shows exemplary pathways and exemplary formation enzymes for the production of HMOs derived from LNTII. Abbreviations: UDP-GlcNAc: Uridine diphosphate N-acetylglucosamine; UDP-Gal: Uridine diphosphate galactose.

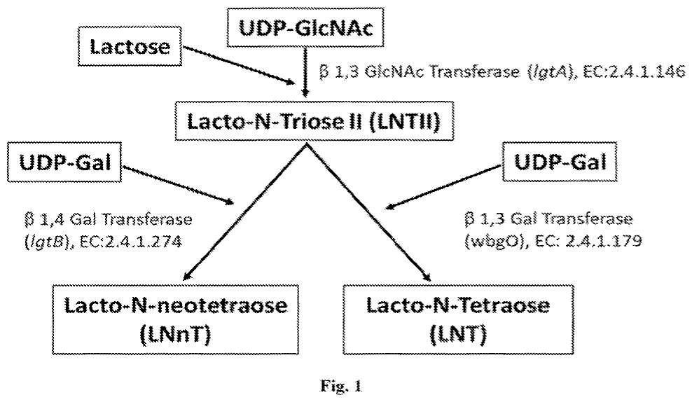

FIG. 2 shows exemplary pathways and exemplary formation enzymes for the production of sialylated HMOs. Abbreviations: GlucNAc: Glucose-N-Acetate=N-Acteyl-Glucosamine; ManNAc: Mannose-N-Acetate=N-Acteyl-Mannosamine; NeuNAc: N acetyl neuraminic acid (Neu5NAc=sialic acid); SL: Sialyl-Lactose

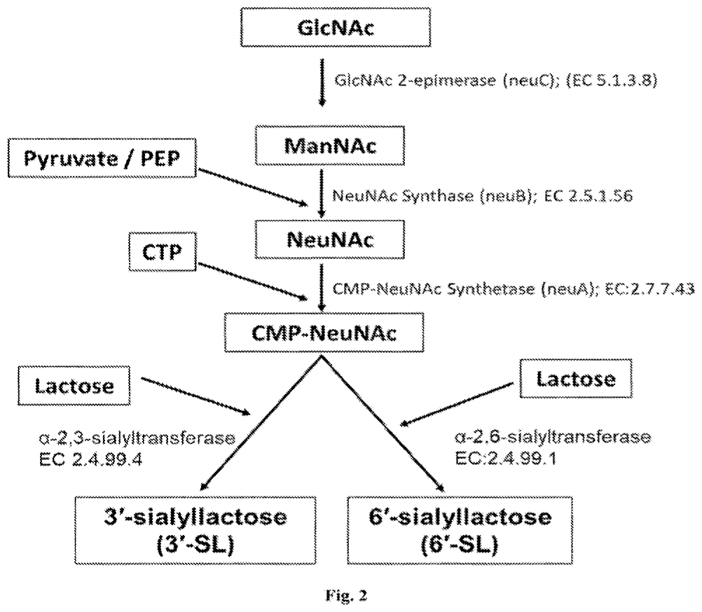

FIG. 3 shows detection of LNnT/LNT. (A) Total ion chromatogram for daughter-ion fragment abundance generated from a 708.3 m/z intact precursor for LNnT/LNT detected by MRM triple quadrupole mass spectrometry. An exemplary sample of the extracellular medium from CDT-1 F335A is shown in grey, LNnT standard is shown in black, and the extracellular medium of a negative control strain lacking CDT-1 is shown as a dashed line. (B) Mass spectra of daughter ion abundance of qualifier (204.0 m/z) and quantifier (366.0 m/z) ions are shown for the CDT-1 F335A extracellular sample and compared to (C) a pure LNnT standard.

FIG. 4 shows detection of 3′-SL. (A) Total ion chromatogram for daughter-ion fragment abundance generated from a 634.2 m/z intact precursor for 3′-SL detected by MRM triple quadrupole mass spectrometry. An exemplary sample of the extracellular medium from codon optimized CDT-1 N209S/F262Y is shown in grey, 3′-SL standard is shown in black, and the extracellular medium of a negative control strain lacking CDT-1 is shown as a dashed line. (B) Mass spectra of daughter ion abundance of qualifier (274.0 m/z) and quantifier (292.0 m/z) ions are shown for the codon optimized CDT-1 N209S/F262Y extracellular sample and compared to (C) a pure 3′-SL standard.

### DETAILED DESCRIPTION

In one aspect, an engineered microorganism capable of producing a human milk oligosaccharide (HMO) is provided. Numerous embodiments are further provided that can be applied to any aspect of the present invention described herein. For example, in some embodiments, the microorganism comprises a first heterologous gene encoding an HMO formation enzyme. In some embodiments, the microorganism further comprises a second heterologous gene encoding a transporter, where the transporter facilitates the export of the produced HMO from the cell. In some embodiments, the transporter is CDT-1 or a variant thereof. In some embodiments, the HMO is a Lacto-N-Triose II (LNTII)-derived HMO or a sialylated HMO. In some embodiments, the HMO is a LNTII-derived HMO, for example lacto-N-neotetraose (LNnT) or lacto-N-tetraose (LNT). In some embodiments, the HMO is a sialylated HMO, for example 3′-sialyllactose (3′-SL) or 6′-sialyllactose (6′-SL).

In some embodiments, the microorganism comprises 1, 2, 3, 4, or more copies of the first heterologous gene. In some embodiments, the microorganism comprises 1, 2, 3, 4, or more copies of the second heterologous gene. The microorganism may further comprise additional heterologous genes. In some embodiments, the microorganism comprises additional heterologous genes encoding one or more additional HMO formation enzymes. In some embodiments, the microorganism comprises additional heterologous genes encoding one or more additional transporters.

In some embodiments, the transporter is a variant of CDT-1. In some embodiments, the CDT-1 has an amino acid sequence of SEQ 1D NO: 4 or a sequence with at least 80%, 85%, 90%, 95%, 98% or 99% homology thereto. In some embodiments, the CDT-1 comprises a PESPR motif (SEQ ID NO: 43). In some embodiments, the CDT-1 comprises a sequence having one or more amino acid replacements at positions corresponding to amino acid positions 91, 209, 213, 256, 262, 335, 411 of SEQ ID NO:4. In some embodiments, the CDT-1 is encoded by a codon optimized nucleic acid. In some embodiments, at least the first 90 nucleotides of the nucleic acid are codon optimized for yeast or at least 5% of the nucleic acid is codon optimized for yeast. In some embodiments, the CDT-1 comprises an amino acid replacement selected from the group consisting of 91A, 209S, 213L, 256V, 262Y, 262W, 335A, 411A and any combination thereof. In some embodiments, the CDT-1 selected from the group consisting of CDT-1 N209S F262Y, CDT-1 G91A, CDT-1 F213L, CDT-1 L256V, CDT-1 F335A, CDT-1 S411A, and CDT-1 N209S F262W, or wherein the CDT-1 comprises an amino acid replacement at a position near the sugar substrate binding pocket and/or the PESPR motif (SEQ ID NO: 43), such as G336, Q337, N341, or G471 In some embodiments, the engineered microorganism utilizes lactose as an HMO substrate. In some embodiments, the variant of CDT-1 is capable of lactose import and HMO export, the variant of CDT-1 has an increased capability of lactose import as compared to CDT-1 (SEQ ID NO: 4), or the variant of CDT-1 has an increased capability of HMO export as compared to CDT-1 (SEQ ID NO: 4). In some embodiments, the engineered microorganism further comprises a genetic modification encoding a second transporter for import of HMO substrate. In some embodiments, the second transporter is lac12 or a variant thereof. In some embodiments, the lac12 has an amino acid sequence of SEQ ID NO: 41 or a sequence with at least 80%, 85%, 90%, 95%, 98% or 99% homology thereto. In some embodiments, the microorganism is selected from the group consisting of an Ascomycetes fungus, a Saccharomyces spp., a Schizosaccharomyces spp., a Pichia spp., Trichoderma, Kluyveromyces, Yarrowia, Aspergillus, and Neurospora. In some embodiments, the HMO formation enzyme is a β 1,3 GlcNAc Transferase or a glycosyltransferase. In some embodiments, the HMO formation enzyme is a β 1,3 GlcNAc Transferase. In some embodiments, the β 1,3 GlcNAc Transferase is encoded by lgtA. In some embodiments, the β 1,3 GlcNAc Transferase has an amino acid sequence selected from SEQ ID NOs: 17-19, 42 or a sequence with at least 80%, 85%, 90%, 95%, 98% or 99% homology thereto. In some embodiments, the HMO formation enzyme is a β 1,3 Gal Transferase. In some embodiments, the β 1,3 Gal Transferase is encoded by wbgO. In some embodiments, the β 1,3 Gal Transferase has an amino acid sequence selected from SEQ ID NOs: 20-22 or a sequence with at least 80%, 85%, 90%, 95%, 98% or 99% homology thereto. In some embodiments, the HMO formation enzyme is a β 1,4 Gal Transferase. In some embodiments, the β 1,4 Gal Transferase is encoded by 103. In some embodiments, the β 1,4 Gal Transferase has an amino acid sequence selected from SEQ ID NOs: 23-25 or a sequence with at least 80%, 85%, 90%, 95%, 98% or 99% homology thereto. In some embodiments, the HMO formation enzyme is a NeuNAc Synthase. In some embodiments, the NeuNAc Synthase has an amino acid sequence selected from SEQ ID NOs: 26-28 or a sequence with at least 80%, 85%, 90%, 95%, 98% or 99% homology. In some embodiments, the HMO formation enzyme is a α-2,6-sialyltransferase. In some embodiments, the α-2,6-sialyltransferase has an amino acid sequence of SEQ ID NO: 34 or a sequence with at least 80%, 85%, 90%, 95%, 98% or 99% homology. In some embodiments, the HMO formation enzyme is a CMP-NeuNAc Synthetase. In some embodiments, the CMP-NeuNAc Synthetase has an amino acid sequence selected from SEQ ID NOs: 29-30 or a sequence with at least 80%, 85%, 90%, 95%, 98% or 99% homology. In some embodiments, the HMO formation enzyme is a α-2,3-sialyltransferase. In some embodiments, the α-2,3-sialyltransferase has an amino acid sequence selected from SEQ ID NOs: 31-33 or a sequence with at least 80%, 85%, 90%, 95%, 98% or 99% homology. In some embodiments, the HMO formation enzyme is a sialyltransferase (PmST). In some embodiments, the sialyltransferase (PmST) has an amino acid sequence of SEQ ID NO: 35 or a sequence with at least 80%, 85%, 90%, 95%, 98% or 99% homology. In some embodiments, the HMO formation enzyme is a UDP-GlcNAc 2-epimerase. In some embodiments, the UDP-GlcNAc 2-epimerase has an amino acid sequence selected from SEQ ID NOs: 36-40 or a sequence with at least 80%, 85%, 90%, 95%, 98% or 99% homology. In some embodiments, the HMO is a sialylated and the HMO formation enzyme is selected from the group consisting of sir 1975 gene from Synechocystis sp. PCC6803, nanA gene from E. coli W3110, neuB gene from E. coli K1, age from Anabaena sp. CH1, neuB from E. coli K12, α-2,3-sialyltransferase gene from Neisseria gonorrhoeae, α-2,6-sialyltransferase from Photobacterium sp. JT-ISH-224, neuC from Campylobacter jejuni, neuB from C. jejuni ATCC 43438, neuA from C. jejuni ATCC 43438, sialyltransferase PmST from Pasteurella multocida, neuB from N. meningitidis MC58 group B, neuC gene from N. meningitidis MC58 group B, Sialidase (Tr6) from Trypanosoma rangeli, alpha-2,3-sialyltransferase from Neisseria meningitidis, NeuNAc Synthase from Campylobacter jejuni, and CMP-NeuNAc Synthetase from Neisseria meningitidis. In some embodiments, the microorganism comprises CMP-NeuNAc Synthetase and α-2,3-sialyltransferase, and wherein the engineered microorganism is capable of producing a sialylated HMO when grown in the presence of sialic acid. In some embodiments, the gene encoding the transporter and the gene encoding the formation enzyme are integrated into the microorganism chromosome. In some embodiments, the gene encoding the transporter and the gene encoding the formation enzyme are episomal. In some embodiments, the microorganism is capable of producing and exporting the HMO. In some embodiments, the CDT-1 is capable of exporting at least 20%, 30%, 40%, 50%, or 60% of the produced HMO. In some embodiments, the microorganism is capable of exporting at least 50% more of the HMO than a parental microorganism lacking the transporter.

In some aspects, the transporter, e.g., CDT-1 or variant CDT-1, includes a leader or targeting sequence for targeting the protein to a particular organelle or location in the cell. For example, the leader/targeting sequence can direct the protein to the cell membrane, the endoplasmic reticulum or the golgi. In some aspects, the leader/targeting sequence is a heterologous sequence (i.e., not part of the native transporter). In some aspects, the leader/targeting sequence directs a portion of the protein to an organelle (e.g., golgi, endoplasmic reticulum) and a portion of the protein is found in a different cellular location, such as the cytoplasmic membrane.

In one aspect, a method of producing an HMO is provided. Numerous embodiments are further provided that can be applied to any aspect of the present invention described herein. For example, in some embodiments, the method comprises providing the engineered microorganism according to those described herein, wherein the engineered microorganism is capable of producing and exporting an HMO, and culturing the engineered microorganism in the presence of a substrate. In some embodiments, a substantial portion of the HMO is exported into the culture medium. In some embodiments, the method further comprises separating the culture medium from the engineered microorganism. In some embodiments, the method further comprises isolating the HMO from the culture medium. In some embodiments, the substrate is selected from the group consisting of lactose, UDP-galactose, Pyruvate/PEP, and CTP. In some embodiments, the transporter is capable of importing lactose and/or exporting the HMO. In some embodiments, the culture medium comprises lactose.

In one aspect, a product suitable for animal consumption is provided. Numerous embodiments are further provided that can be applied to any aspect of the present invention described herein. For example, in some embodiments, the product comprises the microorganism described herein and an HMO produced by the engineered microorganism described herein. In some embodiments, the product further comprises at least one additional consumable ingredient. In some embodiments, the additional consumable ingredient is selected from a protein, a lipid, a vitamin, a mineral or any combination thereof. In some embodiments, the product is suitable for human consumption. In some embodiments, the product is an infant formula, an infant food, a nutritional supplement or a prebiotic product. In some embodiments, the product is suitable for mammalian consumption. In some embodiments, the product is suitable for use as an animal feed. In some embodiments, the product further comprises at least one additional human milk oligosaccharide.

In one aspect, provided herein is an engineered microorganism capable of producing a human milk oligosaccharide (HMO) comprising: a first heterologous gene encoding an HMO formation enzyme and a second heterologous gene encoding a variant of CDT-1, wherein the CDT-1 variant comprises a sequence having one or more amino acid replacements at positions corresponding to amino acid positions 91, 209, 213, 256, 262, 335, 411 of SEQ ID NO:4, or the CDT-1 variant is selected from the group consisting of CDT-1 N209S F262Y, CDT-1 G91A, CDT-1 F213L, CDT-1 L256V, CDT-1 F335A, CDT-1 S411A, and CDT-1 N209S F262W, or the CDT-1 variant comprises an amino acid replacement at a position near the sugar substrate binding pocket and/or the PESPR motif (SEQ ID NO: 43), such as G336, Q337, N341, or G471; and wherein the engineered microorganism produces an HMO and is improved in the uptake of lactose into the microorganism as compared to a parent microorganism that lacks CDT-1, or variant thereof. In some aspects, the HMO is a Lacto-N-Triose II (LNTII)-derived HMO or a sialylated HMO, such as lacto-N-neotetraose (LNnT), lacto-N-tetraose (LNT), 3′-sialyllactose (3′-SL) or 6′-sialyllactose (6′-SL). Numerous embodiments are further provided that can be applied to any aspect of the present invention described herein. For example, in some embodiments, the CDT-1 variant comprises a sequence having one or more amino acid replacements at positions corresponding to amino acid positions 91, 209, 256, 262, 335, 411 of SEQ ID NO:4. In some embodiments, the CDT-1 variant is selected from the group consisting of CDT-1 N209S F262Y, CDT-1 G91A, CDT-1 L256V, CDT-1 F335A, CDT-1 S411A, and CDT-1 N209S F262W. In some embodiments, the HMO is a Lacto-N-Triose II (LNTII)-derived HMO or a sialylated HMO.

### Definitions

For convenience, certain terms employed in the specification, examples, and appended claims are collected here.

As used herein, the singular forms “a,” “an” and “the” are intended to include the plural forms as well, unless the context clearly indicates otherwise.

The term “about” means within an acceptable error range for the particular value as determined by one of ordinary skill in the art, which will depend in part on how the values measured or determined, i.e., the limitations of the measurement system. Where the terms “about” or “approximately” are used in the context of compositions containing amounts of ingredients or conditions such as temperature, these values include the stated value with a variation of 0-10% around the value (X±10%).

The terms “including,” “includes,” “having,” “has,” “with,” or variants thereof are inclusive in a manner similar to the term “comprising.” The term “consisting” and the grammatical variations of consist encompass embodiments with only the listed elements and excluding any other elements. The phrases “consisting essentially of” or “consists essentially of” encompass embodiments containing the specified materials or steps and those including materials and steps that do not materially affect the basic and novel characteristic(s) of the embodiments.

Ranges are stated in shorthand to avoid having to set out at length and describe each and every value within the range. Therefore, when ranges are stated for a value, any appropriate value within the range can be selected, and these values include the upper value and the lower value of the range. For example, a range of two to thirty represents the terminal values of two and thirty, as well as the intermediate values between two to thirty, and all intermediate ranges encompassed within two to thirty, such as two to five, two to eight, two to ten, etc.

The term “genetic modification” as used herein refers to altering the genomic DNA in a microorganism. Typically, a genetic modification alters the expression and/or activity of a protein encoded by the altered gene. A genetic modification encompasses a “variant”, which is a gene or protein sequence that deviates from a reference gene or protein, as further detailed below.

The term “oligosaccharide” refers to saccharide multimers of varying length and includes but is not limited to: sucrose (1 glucose monomer and 1 fructose monomer), lactose (1 glucose monomer and 1 galactose monomer), maltose (1 glucose monomer and 1 glucose monomer), isomaltose (2 glucose monomers), isomaltulose (1 glucose monomer and 1 fructose monomer), trehalose (2 glucose monomers), trehalulose (1 glucose monomer and 1 fructose monomer) cellobiose (2 glucose monomers), cellotriose (3 glucose monomers), cellotetraose (4 glucose monomers), cellopentaose (5 glucose monomers), cellohexaose (6 glucose monomers), 2′-Fucosyllactose (2′-FL, 1 fucose monomer, 1 glucose monomer, and 1 galactose monomer), 3-Fucosyllactose (3′-FL, 1 fucose monomer, 1 glucose monomer, and 1 galactose monomer), 6′-Fucosyllactose (6′-FL, 1 fucose monomer, 1 glucose monomer, and 1 galactose monomer), 3′-Sialyllactose (3′-SL, 1 N-Acetylneuraminic acid monomer, 1 glucose monomer, and 1 galactose monomer), 6′-Sialyllactose (6′-SL, 1 N-Acetylneuraminic acid monomer, 1 glucose monomer, and 1 galactose monomer), Di-fucosyllactose (DF-L, 2 fucose monomers, 1 glucose monomer, and 1 galactose monomer), Lacto-N-triose (LNT II, 1 N-acetylglucosamine monomer, 1 glucose monomer, and 1 galactose monomer), Lacto-N-neotetraose (LNnT, 1 N-acetylglucosamine monomer, 1 glucose monomer, and 2 galactose monomers), Lacto-N-tetraose (LNT, 1 N-acetylglucosamine monomer, 1 glucose monomer, and 2 galactose monomers), Lacto-N-fucopentaose I (LNFP I, 1 fucose monomer, 1 N-acetylglucosamine monomer, 1 glucose monomer, and 2 galactose monomers), Lacto-N-fucopentaose II (LNFP H, 1 fucose monomer, 1 N-acetylglucosamine monomer, 1 glucose monomer, and 2 galactose monomers), Lacto-N fucopentaose III (LNFP III, 1 fucose monomer, 1 N-acetylglucosamine monomer, 1 glucose monomer, and 2 galactose monomers), Lacto-N-fucopentaose IV (LNFP IV, 1 fucose monomer, 1 N-acetylglucosamine monomer, 1 glucose monomer, and 2 galactose monomers), Lacto-N-Fucopentaose V (LNFP V, 1 fucose monomer, 1 N-acetylglucosamine monomer, 1 glucose monomer, and 2 galactose monomers), Lacto-N-fucopentaose VI (LNFP VI, 1 fucose monomer, 1 N-acetylglucosamine monomer, 1 glucose monomer, and 2 galactose monomers), Lacto-N-hexaose (LNH, 2 N-acetylglucosamine monomers, 1 glucose monomer, and 3 galactose monomers), Lacto-N-neohexaose (LNnH, 2 N-acetylglucosamine monomer, 1 glucose monomer, and 3 galactose monomers), Monofucosyllacto-N-hexaose I (MFLNH I, 1 Fucose monomer, 2 N-acetylglucosamine monomer, 1 glucose monomer, and 3 galactose monomers), Monofucosyllacto-N-hexaose II (MFLNH II, 1 Fucose monomer, 2 N-acetylglucosamine monomer, 1 glucose monomer, and 3 galactose monomers), Difucosyllacto-N-hexaose I (LNDFH I, 2 N-acetylglucosamine monomers, 1 glucose monomer, 2 fucose monomers and 3 galactose monomers), Difucosyllacto-N-hexaose II (LNDFH II, 2 N-acetylglucosamine monomers, 1 glucose monomer, 2 fucose monomers and 3 galactose monomers), Difucosyllacto-N-neohexaose (LNnDFH, 2 N-acetylglucosamine monomers, 1 glucose monomer, 2 fucose monomers and 3 galactose monomers), Difucosyl-para-lacto-N-Hexaose (DFpLNH, 2 N-acetylglucosamine monomers, 1 glucose monomer, 2 fucose monomers and 3 galactose monomers), Difucosyl-para-lacto-N neohexaose (DFpLNnH, 2 N-acetylglucosamine monomers, 1 glucose monomer, 2 fucose monomers and 3 galactose monomers), Trifucosyllacto-N-hexaose (TFLNH, 2 N-acetylglucosamine monomers, 1 glucose monomer, 3 fucose monomers and 3 galactose monomers), Sialyllacto-N-neotetraose c (LSTc, 1 N-acetylneuraminic acid monomer, 1 N-acetylglucosamine monomer, 1 glucose monomer, and 2 galactose monomers), Sialyllacto-N-tetraose a (LSTa, 1 N-acetylneuraminic acid monomer, 1 N-acetylglucosamine monomer, 1 glucose monomer, and 2 galactose monomers), Sialyllacto-N-tetraose b (LSTb, 1 N-acetylneuraminic acid monomer, 1 N-acetylglucosamine monomer, 1 glucose monomer, and 2 galactose monomers), Disialyllacto-N-tetraose (DSLNT, 2 N-acetylneuraminic acid monomers, 1 N-acetylglucosamine monomer, 1 glucose monomer, and 2 galactose monomers), FucosylSialyllacto-N-tetraose a (FLSTa, 1 fucose monomer, 1 N-acetylneuraminic acid monomers, 1 N-acetylglucosamine monomer, 1 glucose monomer, and 2 galactose monomers), FucosylSialyllacto-N-tetraose b (FLSTb, 1 fucose monomer, 1 N-acetylneuraminic acid monomers, 1 N-acetylglucosamine monomer, 1 glucose monomer, and 2 galactose monomers), Fucosylsialyllacto-N-hexaose (FSLNH, 1 fucose monomer, 1 N-acetylneuraminic acid monomers, 2 N-acetylglucosamine monomer, 1 glucose monomer, and 3 galactose monomers), Fucosylsialyllacto-N-neohexaose I (FSLNnH I, 1 fucose monomer, 1 N-acetylneuraminic acid monomers, 2 N-acetylglucosamine monomer, 1 glucose monomer, and 3 galactose monomers) and Fucosyldisialyllacto-N-hexaose II (FDSLNH II, 1 fucose monomer, 2 N-acetylneuraminic acid monomers, 2 N-acetylglucosamine monomer, 1 glucose monomer, and 3 galactose monomers).

The terms “human milk oligosaccharide”, “HMO”, and “human milk glycans” refer to oligosaccharides group that are be found in high concentrations in human breast milk. The dominant oligosaccharide in 80% of all women is 2′-fucosyllactose. Other HMOs include 3-fucosyllactose, 6′-fucosyllactose, 3′-sialyllactose, 6′-sialyllactose, di-fucosyllactose, lacto-N-neotetraose, lacto-N-tetraose, lacto-N-fucopentaose I, lacto-N-fucopentaose II, lacto-N-fucopentaose III, lacto-N-fucopentaose IV, lacto-N-fucopentaose V, lacto-N-fucopentaose VI, lacto-N-hexaose, lacto-N-neohexaose, monofucosyllacto-N-hexaose I, monofucosyllacto-N-hexaose II, difucosyllacto-N-hexaose I, difucosyllacto-N-hexaose II, difucosyllacto-N-neohexaose, difucosyl-para-lacto-N-neohexaose, difucosyl-para-lacto-N-hexaose, trifucosyllacto-N-hexaose, sialyllacto-N-neotetraose a, sialyllacto-N-tetraose b, sialyllacto-N-tetraose c, disialyllacto-N-tetraose, fucosylsialyllacto-N-tetraose a, fucosylsialyllacto-N-tetraose b, fucosylsialyllacto-N-hexaose, fucosylsialyllacto-N-neohexaose I, fucosyldisialyllacto-N-hexaose II.

The term “degree of polymerization”, or DP, is the number of monomeric units in a macromolecule or polymer or oligomer molecule.

The term “microorganism” refers to prokaryote or eukaryote microorganisms capable of oligosaccharides production or utilization with or without modifications.

The term, “enhanced utilization” refers to an improvement in oligosaccharide production by a microorganism compared to a parental microorganism, specifically an increase in the oligosaccharides production rate, a decrease in die initial time before oligosaccharides production begins, an increase in the yield, defined as the ratio of product made to the starting material consumed, and/or a decrease in an overall time the microorganisms take to produce a given amount of an oligosaccharide.

The term “parental microorganism” refers to a microorganism that is manipulated to produce a genetically modified microorganism. For example, if a gene is mutated in a microorganism by one or more genetic modifications, the microorganism being modified is a parental microorganism of the microorganism carrying the one or more genetic modifications.

The term, “consumption rate” refers to an amount of oligosaccharides consumed by the microorganisms having a given cell density in a given culture volume in a given time period.

The term, “production rate” refers to an amount of desired compounds produced by the microorganisms having a given cell density in a given culture volume in a given time period.

The term “gene” includes the coding region of the gene as well as the upstream and downstream regulatory regions. The upstream regulatory region includes sequences comprising the promoter region of the gene. The downstream regulatory region includes sequences comprising the terminator region. Other sequences may be present in the upstream and downstream regulatory regions. A gene is represented herein in small caps and italicized format of the name of the gene, whereas, a protein is represented in all caps and non-italicized format of the name of the protein. For example, cdt-1 (italicized) represents a gene encoding the CDT-1 protein, whereas CDT-1 (non-italicized and all caps) represents CDT-1 protein.

The sequence identity of at least 60%, 65%, 70%, 75%, 80%, 85%, 90%, 95%, 96%, 97%, 98%, 99% or 100% to a reference sequence refers to a comparison made between two sequences, preferably using the BLAST algorithm. Algorithms for comparisons between two protein sequences that use protein structural information, such as sequence threading or 3D-1D profiles, are also known in the field.

A “variant” is a gene or protein sequence that deviates from a reference gene or protein. The terms “isoform,” “isotype,” and “analog” also refer to “variant” forms of a gene or a protein. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Suitable amino acid residues that may be substituted, inserted, or deleted, and which are “conservative” or “nonconservative” may be determined by those of skill in the art, including by using computer programs well known in the art.

“Exogenous nucleic acid” refers to a nucleic acid, DNA, or RNA, which has been artificially introduced into a cell. Such exogenous nucleic acid may or may not be a copy of a sequence or fragments thereof which is naturally found in the cell into which it was introduced.

“Endogenous nucleic acid” refers to a nucleic acid, gene, polynucleotide, DNA, RNA, mRNA, or cDNA molecule that is naturally present in a microorganism. An endogenous sequence is “native” to, i.e., indigenous to, the microorganism.

The term “mutation” refers to genetic modification to a gene including modifications to the open reading frame, upstream regulatory region, and/or downstream regulatory region.

A heterologous host cell for a nucleic acid sequence refers to a cell that does not naturally contain the nucleic acid sequence.

A “chimeric nucleic acid” comprises a first nucleotide sequence linked to a second nucleotide sequence, wherein the second nucleotide sequence is different from the sequence which is associated with the first nucleotide sequence in cells in which the first nucleotide sequence occurs naturally.

A constitutive promoter expresses an operably linked gene when RNA polymerase holoenzyme is available. Expression of a gene under the control of a constitutive promoter does not depend on the presence of an inducer.

An inducible promoter expresses an operably linked gene only in the presence of an inducer. An inducer activates the transcription machinery that induces the expression of a gene operably linked to an inducible promoter.

Microorganisms, Systems and Methods for Exporting Human Milk Oligosaccharides

I. Transporters

Provided herein are microorganisms, systems and methods for exporting oligosaccharides such as Human Milk Oligosaccharides (HMOs). In certain aspects, the present disclosure provides genetically engineered microorganisms capable of exporting oligosaccharides. For example, the microorganism described herein can export HMOs, such as lacto-N-neotetraose (LNnT) or lacto-N-tetraose (LNT), such as into the growth medium where the microorganism resides. The HMO may be 3′-sialyllactose (3′-SL) or 6′-sialyllactose (6′-SL).

In some embodiments, the microorganism is genetically engineered to express a transporter that is capable of exporting oligosaccharides from the microorganism. Exemplary transporters include a cellodextrin transporter, which is CDT-1, or homologs and variants thereof.

The transporter CDT-1 from the cellulolytic fungus Neurospora crassa (GenBank: EAA34565.1) belongs to the major facilitator superfamily (MFS) class of transporters capable of transporting molecules comprising hexoses and related carbohydrates. This class of transporters is defined in PFAM under family PF00083 (see the World Wide Web at pfam.xfam.org/family/PF00083).

An example of CDT-1 is provided by the sequence of SEQ ID NO: 4, which is CDT-1 from Neurospora crassa (Uniprot entry Q7SCU1). Homologues of CDT-1 from microorganisms other than N. crassa, particularly, from fungi, can be used in the microorganisms and methods described herein. Non-limiting examples of the homologs of CDT-1 in the instant invention are represented by UniProt entries: A0A0B0E0J3, F8MZD6, G4U961, F7VQY4, Q7SCU1, A0A0J0XVF7, A0A0G2FA71, Q0CVN2, G4T6X5, A0A1Q5T2Z1, A0A0F7VA10, A0A1S9RFP6, A0A0U1LZX5, A0A0C2J3L3, U7PNA2, A0A0F2M9E7, A0A2I1D8G2, A0A2J5HR99, A0A2I2EZ95, A0A0C2IUQ7, U7PNU1, A0A1L7XY52, A0A2J6PQH9, A0A165JU51, A0A167P382, A0A1W2TJP3, A0A175 VST0, A1CN94, S3DBB4, L7IWM4, G4NAG6, L7HX81, G4NAG7, A0A1Y2BF2S, G0SC27, A0A0F7SHM7, A0A2P5HRQ8, A0A194VWR4, A0A194UTG8, B8M4C1, A0A2J6RYZ2, S8AIR7, R9UR53, Q4WR71, B0XPA9, A0A0J5PH40, A0A0K8LME8, A0A1Y2V0X9, A0A0F8VMB5, A1D134, A0A0S7EAY9, A0A2T3AJM0, Q5B9G6, A0A2I1C7L5, A0A167H9D2, A0A2J6SE99, J3PJL4, A0A0C4EGH0, A0A135LD10, A0A0A2I302, A0A0G4NZP3, K9G9B1, K9G7S2, A0A161ZL14, A0A0A2KJ45, A0A136JJM0, and A0A090D3 T9.

An example of CDT-1 is provided by the sequence of SEQ ID NO: 4, which is CDT-1 from Neurospora crassa (Uniprot entry Q7SCU1).

(SEQ ID NO: 4)

| | |
|---|---|
| 1 | MSSHGSHDGA STEKHLATHD IAPTHDAIKI VPKGHGQTAT |
| | KPGAQEEEVR NAALFAAIKE |
| 61 | SNIKPWSKES IHLYFAIFVA FCCACANGYD GSLMTGIIAM |
| | DKFQNQFHTG DTGPKVSVIF |
| 121 | SLYTVGAMVG APFAAILSDR FGRKKGMFIG GIFIIVGSII |
| | VASSSKLAQF VVGRFVLGLG |
| 181 | IAIMTVAAPA YSTEIAPPHW RGRCTGFYNC GWFGGSIEAA |
| | CTTYGCYFIK SNWSWRIPLI |
| 241 | LQAFTCLIVM SSVEFLPESP RFLFANGKDA EAVAFLVKYH |
| | GNGDPNSKLL LLETEEMRDG |
| 301 | IRTDGVDKVW WDYRPLFMTR SGRWRMAQVL MISIFGQFSG |
| | NGLGYENTVI FKNIGVTSTS |
| 361 | QQLAYNILNS VISAIGALTA VSMTDRMPRR AVLIIGTEMC |
| | AAALATNSGL SATLDKQTQR |
| 431 | GTQINLNQGM NEQDAKDNAY LHVDSNYAKG ALAAYFLFNV |
| | IFSPTYTPLQ GVIPTEALET |
| 431 | TIRGKGLALS GFIVNAMGFI NQFAGPIALH NIGYKYIFVP |
| | VGWDLIETVA WYFFGVESQG |
| 541 | RTLEQLEWVY DQPNPVKASL KVEKVVVQAD GHVSEAIVA |

Another example of cellodextrin transporter is CDT-2 from Neurospora crassa (UniProt entry: Q7SD12). CDT-2 is provided by the sequence of SEQ ID NO: 9.

(SEQ ID NO: 9)

| | |
|---|---|
| 1 | MGTFNKKPVA QAVDLNQIQE EAPQFERVDV KKDPGLRKLY |
| | FYAEILCTAS ATTGYDGMFE |
| 61 | NSVQNFETWI KYFGDPRGSE LGLLGALYQT GSIGSIPFVP |
| | LLTDNFGRKT PIIIGCVIMT |
| 121 | VGAVLQATAK NLDTFMGGRT MLGFGNSLAQ IASPMLLTEL |
| | ARPQHRARLT TIYNCLWNVG |
| 181 | ALVVSWLAFG TNYINNDWSW RIPALLQAFP SIIQLLGTWW |
| | VPESPRFLIA KDKHDEALHI |
| 241 | LAKYHANGDP NHPTVQFEFR EIKETIRLEN ESTENSSYLD |
| | FFKSRENRYR LATLLSLGFF |
| 301 | SQWSGNAIIS NYSSKLYETA GVTDSTAKLG LSAGQTGLAL |
| | IVSVIMALNV DKLGRRLAFL |
| 361 | ASTGGMCGTE VIWTLTAGLY GEHRLKGADK AMIFFIWVFG |
| | IFYSLAWSGL LVGYAIEILP |
| 421 | YRLRGKGLMV MNMSVQCALT LNTYANPVAF DYFGPDHSWK |
| | LYLIYTCWIA AEFVFVFEMY |
| 481 | VETRGPTLEE LAKVIDGDEA DVARIDIHQV EKEVEIHEHE |
| | GKSVA |

Other examples of cellodextrin transporter are Cellodextrin transporter cdt-g (UniProt entry: R9USL5), Cellodextrin transporter cdt-d (UniProt entry: R9UTV3). Cellodextrin transporter cdt-c (UniProt entry: R9UR53), Cellodextrin transporter CdtG (UniProt entry: S8A015), Putative Cellodextrin transporter CdtD (UniProt entry: A0A0U5GS76), Cellodextrin transporter CdtC (UniProt entry: S8AIR7), Cellodextrin transporter CdtD (UniProt entry: S8AVE0), and Putative Cellodextrin transporter cdt-c (UniProt entry: A0A0F7VA10).

The UniProt entries listed herein are incorporated by reference in their entireties. Additional homologs of CDT-1 are known in the art and such embodiments are within the purview of the invention. For example, the homologs of CDT-1 have at least 60%, 65%, 70%, 75%, 80%, 85%, 90%, 95%, or 99% sequence identity to SEQ ID NO: 1.

CDT-1 is a substrate-proton symporter from the MFS family. It facilitates the import of beta-1,4-linked disaccharides such as lactose or cellobiose out of the growth medium into the cell. Prior to the discoveries described herein, CDT-1 has been characterized as an importer of substrates such as cellobiose (such as used in the biofuel industry). For example, Ryan et al. (2014) have shown that variants of CDT-1, such as CDT-1 N209S and CDT-1-F262Y have an improved capability to import the oligosaccharide cellobiose. A variant with both mutations CDT-1 N209S/F262Y (or shortly: CDT-1SY) exhibited a further improved uptake of cellobiose. Mapping of the mutations on related MFS transporters revealed that the position N209 of the wildtype CDT-1 is predicted to interact with the oligosaccharide molecule inside the channel. However, neither CDT-1 nor any variants have been shown to be an exporter. To the contrary, outside of the discoveries herein, CDT-1 has been characterized as lacking activity that would provide utility as an exporter (see e.g., Hollands K. et al., Metab Eng. 2019 March; 52:232-242).

CDT-1-N209S/F262Y (or shortly: CDT-ISY)
SEQ ID NO: 1

| | |
|---|---|
| 1 | MSSAGSHDGA STEKHLATHD IAPTNDAIKI VPKGHGQTAT |
| | XPGRQEKEVR NAALFAAIKE |
| 61 | SNIKPWSKES IHLYFAIFVA FCCACANGYD GSLMTGIIAM |
| | DKFQNQFHTG DTGFKVSVIE |
| 121 | SLYTVGAMVG APFAAILSDR FGRKKGMFIG GIFIIVGSII |
| | VABSSKLAQR VVGRFVLGLG |
| 181 | IAIMTVAAPA YSTEIAPPHW RGRCTGFYSC GWFGGSIEAA |
| | CITYGCYFIK SNWSWRIPLI |
| 241 | LOAFTCLIVM SSVFFLPESP RYLFANGRDA EAVAFLVKYH |
| | GNGDPNSKLV LLETEEMRDG |
| 301 | TRTDGVDKVW WDYRPLFMTR SGRWRMAQVL MISTFGQFSG |
| | NGLGYENTVI FKNIGVTSTS |
| 361 | QQLAYNTLNS VISATGALTA VBMTDPMPRR AVLIIGTFMC |
| | AAALATNSGT SATLDKQTQR |
| 421 | GTQINLNQGM NEQDAKDNAY LHVDSNYAKG ALAAXELENV |
| | IFSFTYTPLQ GVIPTEALET |
| 481 | TIRGKGLALS GFTVNAMGFI NQFAGPIALE NTGYKYIFVF |
| | VGWDLIETVA WYFPGVESQG |
| 541 | RTLEQLEWVY DQPNPVKASL KVEKVVVQAD GHVSEAIVA |

CDT-1-N209S (or shortly: CDT-1s):
SEQ ID NO: 2

| | |
|---|---|
| 1 | MSSHGSHDGA STEKHLATHD IAPTHDAIKI VPKGHGQTAT |
| | KFGAQEKEVR NAALFAAIKE |
| 61 | SNIKPWSKES IHLYFAIFVA FCCACANGYD GSLMTGIIAM |
| | DKFQNQFHTG DTGPRVSVIE |
| 121 | SLYTVGAMVG APFAAILSDR FGRKKGMFIG GIFIIVGSII |
| | VASSSKLAQF VVGPFVLGLG |
| 181 | IAIMTVAAPA YSIEIAPPHW RGRCTGFYSC GWFGGSIPAA |
| | CITYGCYFIK SNWSWRIPLI |
| 241 | LQAFTCLIVM SSVFFLPESP RFLFANGRDA EAVAFLVKYH |
| | GNGDPNSKLV LLETEEMRDG |
| 301 | IRTDGVDKVW WDYPPLFMTH SGRWRMAQVL MISIFGQFSG |
| | NGLGYFNTVI FXNIGVTSTS |
| 361 | QQLAYNTLNS VISATGALTA VSMTDRMPRR AVLIIGTFMC |
| | AAALATNSGI SATLDKQTQR |
| 421 | GTQINLNQGM NEQDAKDNAY LHVDSNYAKG ALAAYPLFNV |
| | IFSFTYTPLQ GVIPTEALET |
| 481 | TIRGKGLALS GFIVNAMGFI NQRAGPIALH NTGYKYIFVF |
| | VGWDLIETVA WYTFGVESQG |
| 541 | RTLEQLEWVY DQPNPVKASL KVEKVVVQAD GHVSEAIVA |

CDT-1-F262Y (or shortly: CDT-1y):
SEQ ID NO: 3

| | |
|---|---|
| 1 | MSSBGSHDGA STEKHLATHD IAPTHDATKI VPKGHGQTAT |
| | KPSAQEKEVR NAALFAAIKE |
| 61 | SNIKPWSKES IHLYFAIFVA FCCACANGYD GSLMTGIIAM |
| | DKFQNQFHTG DTGPKVSVIE |
| 121 | SLYTVGAMVG APFAAILSDR FGRKKGMFIG GIPIIVGSII |
| | VASSSKLAQE VVGRPVLGLG |
| 181 | IAIMTVAAPA YSIEIAPPHW RGRCTGFYNC GWFGGSIPAA |
| | CITYSCYFIK SNWSWRIPLI |
| 241 | LQAFTCLIVM SSVFFLPESP RYLFANGRDA EAVAFLVKYH |
| | GNGDPNSKLV LLETEEMRDG |
| 301 | IRTDGVDKVW WDYRPLFMTH SGRWRMAQVL MISIFGQFSG |
| | NGLGYFNTVI FKNTGVTSTS |
| 361 | QQLAYNILNS VISAIGALTA VSMTDRMPRR AVLIIGTEMC |
| | AAALATNSCL SATLDKQTQR |
| 421 | GTQLNLNQGN NEQDAKDNAY LHVDSNYAKG ALAAYFLFNV |
| | IFSETYTPLQ GVTPTEALET |
| 481 | TIRGKGLALS GETVNAMGFI NQFAGPIALH NIGYKYIFVF |
| | VGWDLIETVA WYFFGVESQG |
| 541 | RTLBQLEWVY DQPNPVEASL KVERVVVQAD GHVSEAIVA |

A lactose permease, a membrane protein, is a member of the major facilitator superfamily. Lactose permease can be classified as a symporter, which uses the proton gradient towards the cell to transport β-galactosides such as lactose in the same direction into the cell. In some embodiments, LAC12 is utilized herein as an importer, such that the presence of LAC12 or a variant of lac12 expressed in an engineered microorganism facilitates import of an HMO substrate.

In some embodiments, the engineered microorganism includes an importer that facilitates the import of a substrate into the engineered microorganism such that the substrate can be used for production of an HMO. In some embodiments, the substrate is lactose. In some embodiments, the lactose is imported by the importer LAC 12. Homologues of LAC 12 can be used in the microorganisms and methods described herein. Non-limiting examples of the homologs of LAC12 in the instant invention are represented by UniProt entries: Q9FLB5, B9FJH4, P07921, A0A1J6J8V9, A0A251 TUB0, A0A0A9W318, D0E8H2, W0THP1, A0A1S9RK01, A0A151V9Y9, A0A1C1CDD3, W0TAG2, A0A151W5N5, A0A151VVE7, A0A151 WBL8, A0A151V6X4, A0A151W4U2, A0A1C7LPV6, W0T7D8, W0T8B1, A0A1C1CKJ6, A0A1C1CH50, A0A1C1DO58, A0A1C1C6W6, A0A1C1CIT2, A0A1C1CFR6, A0A2N6NU09, A0A1C1C6I1, A0A1C7LTH2, A0A2N6N8U0, A0A2N6NP59, A0A0F8AZD4, Q8X109, A0A1J6I EJ6, A0A034W1B8, A0A1C7LRQ8, A0A1C1CWY2, A0A1C1CT17, A0A1C1CQ74, A0A1C7M6U6, A0A1C7LT95, A0A2N6NIJ0, A0A2C5X4W3, A0A1C7M1E6, A0A2H8TQZ2, A0A2N6NWY5, A0A1T41ZL8, A0A1T41ZJ1, A0A1T41ZJ3, A0A1T4IZM1, A0A1T4IZL0, A0A1T41ZJ8, A0A0A9YFY8, W8BTJ3, A0A1C7LK22, A0A0C9QF59, and A0A0A9WYQ6.

Other examples of lactose permease are encoded by LacY gene (UniProt entry: P02920, P22733, P47234, P18817, P59832), LacE (UniProt entry: P11162, P24400, P23531, Q4L869, Q5HE15, P50976, Q931G6, Q8CNF7, Q5HM40, Q99S77, Q7A092, Q6GEN9, Q6G7C4, A0A0H3BYW2), LacS gene (UniProt entry: P23936, Q48624, Q7WTB2), LacP (UniProt entry: 033814).

The Uniprot entries listed herein are incorporated by reference in their entireties.

Lactose permease can be expressed in a microorganism and provide lactose uptake. In some aspects, lactose can then be used by the microorganism as a substrate for the production of other oligosaccharides such as HMOs.

Lactose transporter (Lac12)
[Kluyveromyces lactis]
SEQ ID NO: 41

| | |
|---|---|
| 1 | MADHSSSSSS LQKKPINTTE HKDTLGNDRD HKEALNSDND |
| | NTSGLKINGV PTEDAREEVL |
| 61 | LPGYLSKQYY KLYGLCFITY LCATMQGYDG ALMGSTYTED |
| | AYLKYYHLDI NSSSGTGLNF |
| 121 | SIFNVGQICG AFFVPLMDWK GRKPAILIGC LGVVIGAIIS |
| | SLTTTKSALI GGRWFVAFFA |
| 181 | TIANAAAPTY CAEVAPAEDR GKVAGLYNTL WSVGSIVAAF |
| | STYGTNKNFP NSSKAFRIPL |
| 241 | YLQMMFPGLN CIFGWLIPES PRWLVGVGRE EEAREFITKY |
| | HLNGDRTHPL LDMEMARIIE |
| 301 | SFHGTDLSNP LEMLDVRSLE RTRSDRYRAN LVILMAWEGQ |
| | FSGNNVCSYY LPTMLRNVGM |
| 361 | RSVSLNVLMN GVYSIVTWIS SICGAFFIDK IGRREGFLGS |
| | ISGAALALTG LSICTARYEK |
| 421 | TKKRSASNGA LVFIYLFGGI FSFAFTPMOS MYSTEVSTNL |
| | TRSKAQLLNF VVSGVAQFVN |
| 481 | QEATPKAMKN TKYWFYVFYV FFDIFEFTVI YFFFVETKGR |
| | SLEELEVVFE APNPRKASVD |
| 541 | QAFLAQVRAT LVQRNDVRVA NAQNLKEQEP LKSDADNVEK |
| | LSEAESV |

As described herein, a cellobiose transporter acting as an importer within Neurospora crassa can act as an exporter when expressed in a microorganism such as when expressed in Saccharomyces cerevisiae strains producing an HMO. In some embodiments, the HMO exported by such transporter is a non-branched HMO comprised of a lactose core with modifications to the galactose ring. In some embodiments, the HMO is 3′-sialyllactose (3′-SL), 6′-sialyllactose (6′-SL), lacto-N-neotetraose (LNnT), lacto-N-tetraose (LNT), Lacto-N-Triose II (LNTII)-derived HMO or sialylated HMO or any combinations thereof. In some embodiments, the HMO is Lacto-N-Triose II (LNTII)-derived HMO or sialylated HMO. The HMO may be 3′-sialyllactose (3′-SL) or 6′-sialyllactose (6′-SL).

In some embodiments, the transporter for export of HMOs is a CDT-1 or homolog thereof. In some embodiments, the transporter for export of HMOs is a variant, such as a mutant CDT-1, where one or more amino acids are altered as compared to a CDT-1 amino acid sequence. In some embodiments, a mutant CDT-1 for exporting HMOs comprises an amino acid sequence of SEQ ID NO: 1 or an amino acid sequence having 80%, 85%, 90%, 95%, 98%, 99% or greater than 99% homology with SEQ ID NO: 1. The mutant CDT-1 can have one or more amino acid changes that correspond to one or more of positions 91, 209, 213, 256, 262, 335, and 411 of SEQ ID NO: I. The mutant CDT-1 can comprise SEQ ID NO:1 having one or more amino acid substitutions selected from G91A, N209S, F213L, L256V, F262Y, F262W, F335A, S411A. In some embodiments, the mutant CDT-1 is CDT-1 N209S F262Y (SEQ ID NO: 1), CDT-1 G91A (SEQ ID NO: 10), CDT-1 F213L (SEQ ID NO: 11), CDT-1 L256V (SEQ ID NO: 12), CDT-1 F335A (SEQ ID NO: 13), CDT-1 S411A (SEQ ID NO: 14), or CDT-1 N209S F262W (SEQ ID NO: 15). The CDT transporter, such as a CDT-1 or mutant CDT-1 when expressed in a microorganism exports HMO such as Lacto-N-Triose II (LNTII)-derived HMO or sialylated HMO. For example, cdt-1sy gene (encoding CDT-1 N209S/F262Y) is expressed within a background strain (microorganism) producing Lacto-N-Triose II (LNTII)-derived HMO or sialylated HMO and Lacto-N-Triose II (LNTII)-derived HMO or sialylated HMO accumulation in the growth medium during a fermentation experiment is compared to the same strain without the cdt-1-sy gene. The expression of CDT-1 N209S/F262Y increases the accumulation of Lacto-N-Triose II (LNTII)-derived HMO or sialylated HMO within the growth medium indicating that CDT-1 SY can act as an efficient substrate exporter.

| Lactose permease mutant (CDT-1 G91A) | |
|---|---|
| [Neurosporacrassa] | |
| SEQ ID NO: 10 | |
| 1 | MSSHGSHDGA STEKHLATHD IAPTHDAIKI VPKGHGQTAT KPGAQEKEVP NAALEAAIKE |
| 61 | SNIKPWSKES IRLYFAIFVA FCCACANGYD ASLMTGIIAM DEFQNQFHTG DTGPRVSVIF |
| 121 | SLYTVGAMVG APFAAILSDR FGRKKGMFIG GIFIIVGSII VASSSKLAQF VVGRFVLGLG |
| 181 | IAIMTVAAPA YSIETAPPHW RGRCTGFYNC GWFGGSIPAA CITYGCYFTK SNWSWRIPLI |
| 241 | LQAFTCLIVM SSVFFLFESP RFLFANGRDA EAVAFLVKYH GNGDPNSKLV LLETEEMRDG |
| 301 | IRTDGVDEVW WDYRPLTMTH SGRWRMAQVL MISIFGQFSG NGLGYFNTVI FKNIGVTSTS |
| 361 | QQLAYNTLNS VISAIGALTA VSMTDRMPRR AVLIIGTFMC AAALATNSGL SATLDEGTQR |
| 421 | GTQINLNQGM NEQDAKDNAY LHVDSNYARG ALAAYFLFNV IFSFTYTPLQ GVIFTEALET |
| 481 | TTRGKGLALS GFIVNAMGFI NQFAGPTALH NTGYKYIFVF VGWDLIETVA WYFFGVESQG |
| 541 | RTLEQLEWVY DQPNPVKASE KVEKVVVQAD GHVSEAIVA |
| Lactose permease mutant (CDT-1 F213L) | |
| [Neurosporacrassa] | |
| SEQ ID NO: 11 | |
| 1 | MSSHGSHDGA STEKHLATHD TAPTHDAIKT VPKGHGQTAT KPGAQEKEVR NAALEAAIKE |
| 61 | SNIKPWSKES IHTYFAIFVA FCCACANGYD GSLMTGIIAM DKFQNQFRTG DTGPKVSVIE |
| 121 | SLYTVGAMVG APFAAILSDR FGRKKGMFTG GIFTIVGSII VASSSKLAQF VVGRFVLGLG |
| 181 | IAIMTVAAPA YSTEIAPPHW RGRCTGFYNC GWLGGSIPAA CITYGCYFTK SNWSWRIPLI |
| 241 | LQAFTCLIVM SSVFFLPESP RFLFANGRDA EAVAFLNKYH GNGDPNSKLV LLETEEMRDG |
| 301 | IRTDGVDKVW WDYRPLFMTR SGRWRMAQVL MISIFGQFSG NGLGYFNTVI FKNIGVTSTS |
| 361 | QQLAYNILNS VISAIGALTA VSMTDRMPRR AVLIXGTEMC AAALATNSGL SATLDKQTOR |
| 421 | GTQINLNQGM NEQDAKDNAY LHVDSNYAKG ALAAYFLFNV IFSFTYTPLQ GVIPTEALET |
| 481 | TIRGKGLALS GFIVNAMGFI NQFAGPIALH NIGYKYIFVF VGWDLIETVA WYFFGVESQG |
| 541 | RTLEQLEWVY DQPNPVKASL KVEKVVVQAD GHVSEAIVA |
| Lactose permease matant (CDT-1 L256V) | |
| [Nearospora crassa] | |
| SEQ ID NO: 12 | |
| 1 | MSSHGSHDGA STEKHLATHD IAPTHDAIKI VPKGHGQTAT KPGAQEKEVR NAALFAAIKE |
| 61 | SNIKPWSKES IRLYFAIFVA FCCACANGYD GSLMTGIIAM DRFQNQFHTG DTGPRVSVIE |
| 121 | SLYTVGAMVG APFAAILSDR FGRKKGMFIG GIFIIVGSII VASSSKXAQF VVGRFVLGLG |
| 181 | IAIMTVAAPA YSTEIAPPHW RGRCTGFYNC GWFGGSIPAA CTTYGCYFIK SNWSWRIPLI |
| 241 | LQAFTCLTVM SSVFFVPESP RFLFANGRDA EAVAFLVKYH GNGDPNSKLV LLETEENRDG |
| 301 | IRTDGVDKVW WDYRPLFMTR SGRWRMAQVL MISIFGQFSG NGLGYENTVI FKNIGVTSTS |
| 361 | QQLAYNILNS VISAIGALTA VSMTDRMPRR AVLIIGTFMC AAALATNSGL SATLDKQTQR |
| 421 | GTQINLNQGM NEQDAKDNAY LHVDSNYAKG ALAAYFLENV IFSFTYTPLQ GVIPTEALET |
| 481 | TIRGKGLALS GFIVNAMGFI NQFAGPIALH NIGYKYIFVP VGWDLIETVA WYFPGVESOG |
| 541 | RTLEGLEWVY DQPNPVKASL KVEKVVVQAD GHVSEAIVA |
| Lactose permease mutant (CDT-1 F335A) | |
| [Neurospora crassa] | |
| SEQ ID NO: 13 | |
| 1 | MSSHGSHDGA STEKHLATHD IAPTHDAIKI VPKGHGQTAT KPGAQEKSVR NAALFAAIKE |
| 51 | SNIKPWSKES THLYFAIFVA FCCACANGYD GSIMTGIIAM DKFONQFHTG DTGPKVSVIF |
| 121 | SLYTVGAMVG APFAAILSDR FGRKKGMFIG GTFIIVGSII VASSSKLAQF VVGRFVLGLG |
| 181 | IAIMTVAAPA YSIEIAPEHW RGRCTGFYNC GWFGGSIPAA CTTYGCYFIK SNWSWRIPLI |
| 241 | LQAFTCLIVM SBVFFLPESP RFLFANGRDA EAVAFLVKYH GNGDPNSKLV LLETEEMRDG |
| 301 | IRTDGVDKVW WDYRPLFMTH SGRWRMAQVL MTSIAGQFSG NGLGYFNTVI FKNIGVTSTS |
| 361 | QQLAYNILNS VISAIGALTA VSMTDRMPRR AVLIIGTFMC AAALATNSGL SATLDKQTQR |
| 421 | GTQINLNQGM NEQDAKDNAY LHVDSNYAKG ALAAYFLFNV IFSFTYTPLQ GVIPTEALET |
| 481 | TIRGKGLALS GFTVNAMGFT NQFAGPIALH NIGYKYIFVF VGWDLIETVA WYFFGVESOG |
| 541 | RTLEQLEWVY DQPNPVKASL KVEKVVVQAD GHVSEAIVA |
| lactose permease mutant (CDT-1 S411A) | |
| [Neurospora crassa] | |
| SEQ ID NO: 14 | |
| 1 | MSSHGSHDGA STEKHLATHD IAPTHDATKI VPKGHGQTAT KPGAQEKEVR NAALFAAIKE |
| 61 | SNTKPWSKES THLYFAIFVA FCCACANGYD GSLMTGIIAM DKRQNQFRTG DTGPKVSVIF |
| 121 | SLYTVGAMVG APFAAILSDR FGRKKGMFIG GIPIIVGSII VASSSKLAQF VVGRFVLGLG |
| 181 | IAIMTVAAPA YSIEIAPPHW RGRCTGFYNC GWFGGSIPAA CTTYGCYFIK SNWSWRIPLI |
| 241 | LQAETCLIVM SSVFFLPESP RFLFANGRDA EAVAFLVKYH GNGDPNSKLV LLETEEMRDG |
| 301 | IRTDGVDKVW WDYRPLFMTH SGRWRMAQVL MXSIFGQFSG NGLGYFNTVI FKNIGVTSTS |
| 361 | QQLAYNILNS VISAIGALTA VSMTDRMPRR AVLIIGTFMC AAALATNSGL AATLDKQTQR |
| 421 | GTQINLNQGM NEQDAKDNAY LHVDSNYAKG ALAAYFLFNV IFSFTYTPLQ GVIPTEALET |
| 481 | TIRGKGLALS GFTVNAMGFT NQFAGPIALH NIGYKYIFVF VGWDLIETVA WYFFGVESQG |
| 541 | RTLEQLEWVY DQPNPVKASL KVEKVVVQAD GHVSEAIVA |
| lactose permease mutant (CDT-1 N209S F252W) | |
| [Neurospora crassa] | |
| SEQ ID NO: 15 | |
| 1 | MSSHGSHDGA STEKHLATHD IAPTRDAIKI VPKGHGQTAT KPGAQEKEVR NAALFAAIKE |
| 61 | SNIKPWSKES IHLYFAIFVA FCCACANGYD GSLMTGITAM DKFQNQFHTG DTGPKVSVIE |
| 121 | SLYTVGAMVG APFAAILSDR FGRKKGMFIG GIFIIVGSII VASSSKLAQF VVGRFVLGLG |
| 181 | IALMTVAAPA YSIEIAPPHW RGRCTGFYSC GWFGGSIPAA CTTYGCYFIK SNWSWRIPLI |
| 241 | LQAPTCLIVM SSVFFLPESP RWLEANGRDA EAVAFLVKYH GNGDPNSKLV LLETEEMRDG |
| 301 | IRTDGVDKVW WDYRPLFMTA SGRWRMAQVL MXSIFGQFSG NGLGYFNTVI FKNIGVTSTS |
| 361 | QQLAYNILNS VISAIGALTA VSMTDRMPRR AVLIIGTFMC AAALATNSGL SATLDKQTQR |
| 421 | GTQINLNQGM NEQDAKDNAY LHVDSNYAKG ALAAYFLFNV IFSFTYTPLQ GVIPTEALET |
| 481 | TIRGKGLALS GFTVNAMGFI NQFAGPIALH NJGYKYIFVF VGWDLIETVA WYFFGVESQG |
| 541 | RTLEQLEWVY DQPNPVEAST EVEKVVVQAD GEVSEAIVA |
| lactose permease mutant (CDT-1 209S 262Y first | |
| 30 amino acid codons optimized by yeast) | |
| [Neurospora crassa] | |
| SEQ ID NO: 16 | |
| 1 | ATGTCCTCYC ATGGTTCTCA TGATGGTGCT TCTACTGAAA AACATTTGGC CACTCATGAT |
| 61 | ATTGCTCCAA CTCATGATGC TATCAAGATC GTGCCCAAGG GCCATGGCCA GACAGCCACA |
| 121 | AAGCCCGGCG CCCAAGAGAA GGAGGTCCGC AACGCCGCCC TATTTGCGGC CATCAAGGAG |
| 181 | TCCAATATCA AGCCCTGGAG CAAGGAGTCC ATCCACCTOT ATTTCGCCAT CTTCGTCGCC |
| 241 | TTPTGTTGTG CATGCGCCAA CGGTTACGAT GGTTCACTCA TGACCGGAAT CATCGCTATG |
| 301 | GACAAGTECC AGAACCAATT CCACACTGGE GACACTGGTC CTAAAGPCTC PGTCATCTTT |
| 361 | TCTCTCTATA CCGTGGGTGC CATGCTTGGA GCTCCCTTCG CTGCTATCCT CTCTGATCGT |
| 421 | TTTGGCCGTA AGAAGGGCAT GTTCATCGGT GGTATCTTTA TCATTGTCGG CTCCATTATT |
| 481 | GFPGCTAGCT CCTCCAAGCT CGCTCAGTPT GTCGTTGGCC GCTTCGTTCT TGGCCTCGGT |
| 541 | ATCGCCATCA TGAGCGTTGC TGCCCCGGCC TACPCCATCG AAATOGCCCC TCCTGACYGG |
| 601 | ceceGccecr GCACTGGCTT CTACAgCTGC GGTTGGTTCG GAGGTTCGAT TCCTGCCGCC |
| 661 | TGCATCACCT ATGGCTGCTA CTTCATTAAG AGCAACTGGT CATGGCGTAT CCCCTTGATC |
| 721 | CTTCAGGCTT TCACGTGCCT TATCGTCATG TCCTCCGTCT TCTTCCTCCC AGAATCCCCT |
| 781 | CGCTACCTAT TTGCCAACGG CCGCGACGCT GAGGCTGTTG CCTTTCTTGT CAAGTATCAC |
| 841 | GGCAACGGCG ATCCCAATTC CAAGCTGGTG TTGCTCGAGA CTGAGGAGAT GAGGGACGGT |
| 901 | ATCAGGACCG ACGGTGTCGA CAAGGTCTOG TGGGATTACC GCCCGCTCTT CATGACCCAC |
| 961 | AGCGGCCGCT GGOGCATOGC CCAGGTGCTC ATGATCTCCA TCTTTGGCCA GTTCTCCGGC |
| 1021 | AACGGTCTOG GTTACTTCAA TACCGTCATC TTCAAGAACA TTGGTETCAG CAGCACCTCC |
| 1081 | CAACAGCTCG CCTACAACAT CCTCAACTCC GTCATCTCCG CTATCGGTGC CTTGACCGCC |
| 1141 | GTCTCCATGA CTGATCGTAT GCCCCGCCGC GCGGTGCTCA TTATCGGTAC CTTCATGTGC |
| 1201 | GCCGCTGCTC TTGCCACCAA CTCGGGTCTT TCGGCTACTG TCGACAAGCA GACTCAAAGA |
| 1261 | GGCACGCAAA TCAACCTGAA CCAGGGTATG AACGAGCAGG ATGCCAAGGA CAACSCCTAC |
| 1321 | CTCCACGTCG ACAGCAACTA CGCCAAGGGT GCCCTGGCCG CTTACTTCCT CTTCAACGTC |

| | |
|---|---|
| 1381 | ATCTTCTCCT TCACCTACAC TCCCCTCCAG GGTGTTATTC CCACCGAGGC TCTCGAGACC |

| | |
|---|---|
| 1441 | ACCATCCGTG GCAAGGGTCT TGCCCTTTCC GGCTTCATTG TCAACGCCAT GGGCTTCATC |
| 1501 | AACCAGTTCG CTGGCCCCAT CGCTCTCCAC AACATTGGCT ACAAGTACAT CTTTGTCTTT |
| 1561 | GTCGGCTGGG ATCTTATCGA GACCGTCGCT TGGTACTTCT TTGGTGTCGA ATCCCAAGGC |
| 1621 | CGTACCOTCG AGCAGCTCGA ATGGGTCTAC GACCAGCCCA ACCCCGTCAA GGCCTCCCTA |
| 1681 | AAAGTOEAAA AGGTCGTCGP CCAGGCCGAC GGCCAPGTGT CCGAAGCTAT CGTTGCTTAA |

In some embodiments, a variant of CDT-1 and related transporters for use as an HMO exporter can include one or more mutations of amino acids predicted to be near the sugar substrate binding pocket (e.g., N209S in CDT-1) or near the highly-conserved PESPR motif (SEQ ID NO: 43) in the sugar porter family PF00083 (e.g., F262Y in CDT-1). Exemplary mutations include amino acids in CDT-1 predicted to be in the substrate binding pocket such as G336, Q337, N341, and G471.

In some embodiments, modifications of a microorganism expressing a transporter such as CDT-1 or a CDT-1 mutant can be engineered to increase the activity of the transporter. Non-limiting examples of genetic modifications to cdt-1 that can increase the activity of CDT-1 as a substrate exporter in the microorganisms compared to CDT-1 substrate import activity in the parental microorganisms include one or more of: a) replacement of an endogenous promoter with an exogenous promoter operably linked to the endogenous cdt-1; b) expression of a cdt-1 via an extrachromosomal genetic material; c) integration of one or more copies of cdt-1 into the genome of the microorganism; d) a modification to the endogenous cdt-1 to produce a modified CDT-1 that encodes a transporter protein that has an increased activity as a substrate exporter; e) introduction into the microorganism on extrachromosomal genetic material comprising a cdt-1 or a variant of cdt-1 (mutant cdt-1) such as encoding CDT-1 N209S F262Y or one or more of the variants described herein (e.g., CDT-1 G91A, CDT-1 F213L, CDT-1 L256V, CDT-1 F335A, CDT-1 S411A, or CDT-1 N209S F262W); f) integration into the genome of the microorganism of one or more copies of cdt-1 or a variant of cdt-1 encoding a transporter such as CDT-1 N209S F262Y, CDT-1 G91A, CDT-1 F213L, CDT-1 L256V, CDT-1 F335A, CDT-1 S411A, or CDT-1 N209S F262W; (g) introduction through extrachromosomal genetic material or through integration of a variant of cdt-1 encoding CDT-1 with one or more mutations of amino acids predicted to be near the sugar substrate binding pocket and/or the PESPR motif (SEQ ID NO: 43) such as positions G336, Q337, N341, and G471; and/or (h) codon optimization of part of or all of cdt-1 or a variant of cdt-1.

Any combinations of the modifications (a) to (h) described in this paragraph are also envisioned. In some embodiments, an expression of cdt-1 or its variants is varied by utilizing different promoters or changes immediately adjacent to the introduced cdt-1 gene. For example, in certain embodiments the deletion of a URA3 cassette adjacent to an introduced cdt-1sy expression cassette leads to a further improvement of HMO export, such as lacto-N-neotetraose (LNnT) or lacto-N-tetraose (LNT) export. The HMO may be 3′-sialyllactose (3′-SL) or 6′-sialyllactose (6′-SL).

In some embodiments, the endogenous promoter is replaced with an exogenous promoter that induces the expression of cdt-1 at a higher level than the endogenous promoter. In certain embodiments, the exogenous promoter is specific for the microorganism in which the exogenous promoter replaces the endogenous promoter. For example, a yeast specific exogenous promoter can be used if the microorganism being modified is a yeast. The exogenous promoter can be a constitutive promoter or inducible promoter.

Non-limiting examples of constitutive yeast specific promoters include: pCYC1, pADH1, pSTE5, pADH1, pCYC100 minimal, pCYC70 minimal, pCYC43 minimal, pCYC28 minimal, pCYC16, pPGK1, pCYC, pGPD or pTDH3. Additional examples of constitutive promoters from yeast and examples of constitutive promoters from microorganisms other than yeast are known to a skilled artisan and such embodiments are within the purview of the invention.

Non-limiting examples of inducible yeast specific promoters include: pGAL1, pMFA1, pMFA2, pSTE3, pURA3, pFIG1, pENO2, pDLD, pJEN1, pmCYC, and pSTE2. Additional examples of inducible promoters from yeast and examples of inducible promoters from microorganisms other than yeast are known to a skilled artisan and such embodiments are within the purview of the invention.

In certain embodiments, the microorganisms comprise a modification to the wildtype cdt-1 to produce a modified cdt-1 that encodes a transporter with an increased capability to export Lacto-N-Triose II (LNTII)-derived HMO or sialylated HMO from the cell.

Accordingly, in certain embodiments, modification of the wildtype cdt-1 produces a modified cdt-1 that encodes a CDT-1 with increased export rates of Lacto-N-Triose II (LNTII)-derived HMO or sialylated HMO. In certain embodiments, wildtype cdt-1 is mutated around the conserved PESPR motif (SEQ ID NO: 43) which is conserved in hexose transporters. In certain embodiments, cdt-1 is modified leading to the production of a protein CDT-1-F262Y. The mutant CDT-1 can have one or more amino acid changes that correspond to one or more of positions 91, 209, 213, 256, 262, 262, 335, and 411 of SEQ ID NO:1. The mutant CDT-1 can comprise SEQ ID NO: 1 having one or more amino acid substitutions selected from G91A, N209S, F213L, L256V, F262Y, F262W, F335A, S411A. In some embodiments, the mutant CDT-1 is CDT-1 N209S F262Y, CDT-1 G91A, CDT-1 F213L, CDT-1 L256V, CDT-1 F335A, CDT-1 S411A, or CDT-1 N209S F262W. The mutant CDT-1 can have one or more amino acid changes that correspond to one or more of positions predicted to be near the sugar substrate binding pocket and/or the PESPR motif (SEQ ID NO: 43) such as positions G336, Q337, N341, and G471.

In certain embodiments wild-type cdt-1 is mutated around the amino acid residues within CDT-1 which are interacting with the oligosaccharide substrate. In certain embodiments cdt-1 is modified leading to the production of a protein CDT-1-N209S. In yet other embodiments cdt-1 is modified leading to the production of a protein CDT-1-N209S F262Y. In some certain embodiments cdt-1 is modified leading to the production of a protein CDT-1 G91A. In some certain embodiments cdt-1 is modified leading to the production of a protein CDT-1 F213L. In some certain embodiments cdt-1 is modified leading to the production of a protein CDT-1 L256V. In some certain embodiments cdt-1 is modified leading to the production of a protein CDT-1 F335A. In some certain embodiments cdt-1 is modified leading to the production of a protein CDT-1 S411A. In some certain embodiments cdt-1 is modified leading to the production of a protein CDT-1 N209S F262W.

In specific embodiments, a microorganism, preferably, a fungus such as a yeast, preferably, a Saccharomyces spp., and preferably, S. cerevisiae is provided, the microorganism comprising the genetic modifications or the combinations of genetic modifications listed below:
1) A genetic modification producing a CDT-1 conferring the cell with oligosaccharide-, and in particular, HMO-export activity, such as lacto-N-neotetraose (LNnT) or lacto-N-tetraose (LNT)-export activity. The HMO may be 3′-sialyllactose (3′-SL) or 6′-sialyllactose (6′-SL).
2) A genetic modification producing a CDT-1 with mutated amino acid residues increasing export activity of CDT-1 for oligosaccharides, HMO-export activity, such as and in particular lacto-N-neotetraose (LNnT) or lacto-N-tetraose (LNT). The HMO may be 3′-sialyllactose (Y-SL) or 6′-sialyllactose (6′-SL).

In some embodiments, the microorganisms provided herein are engineered to express CDT-1 with one or more mutated amino acid residues and such microorganisms are altered in their uptake of lactose as compared to a parent microorganism (e.g., as compared to the microorganism not containing a CDT-1 or CDT-1 variant or as compared to the microorganism engineered to express the nonmutated (wildtype) form of CDT-1). In some aspects, the engineered microorganism is increased in lactose uptake as compared to the parent microorganism. In some embodiments, the engineered microorganism is decreased in lactose uptake as compared to the parent microorganism. In some aspects, the microorganism engineered with the CDT-1 variant also can be altered in its HMO-export activity as compared to a parent microorganism. In some aspects, the microorganism is engineered with a CDT-1 variant where the mutated amino acid corresponds to one or more of positions 91, 209, 213, 256, 262, 262, 335, and 411 of SEQ ID NO:1. The CDT-1 variant can comprise SEQ ID NO:1 having one or more amino acid substitutions selected from G91A, N209S, F213L, L256V, F262Y, F262W, F335A, S411A. In some embodiments, the mutant CDT-1 is CDT-1 N209S F262Y, CDT-1 G91A, CDT-1 F213L, CDT-1 L256V, CDT-1 F335A, CDT-1 S411A, or CDT-1 N209S F262W. The CDT-1 variant can have one or more amino acid changes that correspond to one or more of positions predicted to be near the sugar substrate binding pocket and/or the PESPR motif (SEQ ID NO: 43) such as positions 0336, Q337, N341, and G471. In some aspects, the CDT-1 variant does not have a mutation at position 213.

II. Formation Enzymes

Provided herein are microorganisms, systems and methods for producing and exporting oligosaccharides such as Human Milk Oligosaccharides (HMOs). In certain aspects, the present disclosure provides genetically engineered microorganisms capable of exporting oligosaccharides. For example, the microorganism described herein can export HMOs, such as lacto-N-neotetraose (LNnT) or lacto-N-tetraose (LNT), such as into the growth medium where the microorganism resides. The HMO may be 3′-sialyllactose (3′-SL) or 6′-sialyllactose (6′-SL).

In some embodiments, the microorganism is genetically engineered to express one or more formation enzymes that are capable of producing oligosaccharides that arc not naturally present in the microorganism, or not naturally present at high levels. Exemplary formation enzymes include β 1,3 GlcNAc Transferase, β 1,3 Gal Transferase, β 1,4 Gal Transferase, NeuNAc Synthase, CMP-NeuNAc Synthetase, α-2,6-sialyltransferase, α-2,3-sialyltransferase, sialyltransferase (PmST), and UDP-GlcNAc 2-epimerase, or homologs and variants thereof. Other examples of formation enzymes are encoded by genes including slr1975 gene from Synechocystis sp. PCC6803, nanA gene from E evil W3110, neuB gene from E. coli K1, age from Anabaena sp. CH1, neuB from E. coli K12, α-2,3-sialyltransferase gene from Neisseria gonorrhoeae, α-2,6-sialyltransferase from Photobacterium sp. JT-ISH-224, neuC from Campylobacter jejuna, neuB from C. jejuni ATCC 43438, neuA from C. jejuna ATCC 43438, sialyltransferase PmST from Pasteurella multocida, neuB from N. meningitidis MC58 group B, neuC gene from N. meningitidis MC58 group B, Sialidase (Tr6) from Trypanosoma rangeli, alpha-2,3-sialyltransferase from Neisseria meningitidis, NeuNAc Synthase from Campylobacter jejuni, and CMP-NeuNAc Synthetase from Neisseria meningitides.

β-1,3-N-acetylglucosaminyltransferase (β 1,3 GlcNAc Transferase) is an enzyme involved in the synthesis of poly-N-acetyllactosamine and catalyzes the initiation and elongation of poly-N-acetyllactosamine chains. In some embodiments, the β 1,3 GlcNAc Transferase is encoded by lgtA gene. Non-limiting examples of β 1,3 GlcNAc Transferase are an amino acid sequence selected from: SEQ ID NOs: 17-19 or a sequence with at least 80%, 85%, 90%, 95%, 98% or 99% homology thereto.

β 1,3 galactosyltransferase (β 1,3 Gal Transferase) is an enzyme that transfers galactose from UDP-galactose to substrates with a terminal beta-N-acetylglucosamine (beta-GlcNAc) residue. It is also involved in the biosynthesis of the carbohydrate moieties of glycolipids and glycoproteins. In some embodiments, the β 1,3 Gal Transferase is encoded by wbgO gene. Non-limiting examples of β1,3 GlcNAc Transferase are an amino acid sequence selected from: SEQ ID NOs: 20-22 or a sequence with at least 80%, 85%, 90%, 95%, 98% or 99% homology thereto.

β-1,4-galactosyltransferase (β 1,4 Gal Transferase) catalyzes the production of lactose in the lactating mammary gland and could also be responsible for the synthesis of complex-type N-linked oligosaccharides in many glycoproteins as well as the carbohydrate moieties of glycolipids. In some embodiments, the β 1,4 Gal Transferase is encoded by lgtB gene. Non-limiting examples of β1,4 Gal Transferase are an amino acid sequence selected from: SEQ ID NOs: 23-25 or a sequence with at least 80%, 85%, 90%, 95%, 98% or 99% homology thereto.

N-acetylneuraminate (NeuNAc) Synthase is an enzyme that functions in the biosynthetic pathways of sialic acids. Non-limiting examples of NeuNAc Synthase are an amino acid sequence selected from: SEQ ID NOs: 26-28 or a sequence with at least 80%, 85%, 90%, 95%, 98% or 99% homology thereto.

Cytidine monophosphate N-acetylneuraminic acid synthetase (CMP-NeuNAc Synthetase) converts N-acetylneuraminic acid (NeuNAc) to cytidine 5′-monophosphate N-acetylneuraminic acid (CMP-NeuNAc). This process is important in the formation of sialylated glycoprotein and glycolipids. Non-limiting examples of CMP=NeuNAc Synthetase are an amino acid sequence selected from: SEQ ID NOs: 29-30 or a sequence with at least 80%, 85%, 90%, 95%, 98% or 99% homology thereto. In some embodiments herein, the genetically engineered microorganism capable of exporting oligosaccharides has one or more pathway enzymes and produces CMP-NeuNAc. In some aspects, the genetically engineered microorganism further includes an enzyme to produce a sialyllactose from the CMP-NeuNAc. In some aspects, sialyllactose is 3′SL and/or 6′SL.

α-2,3-sialyltransferase transfers a sialic acid moiety from cytidine-5′-monophospho-N-acetyl-neuraminic acid (CMP-NeuAc) to terminal positions of various key glycoconjugates, which play critical roles in cell recognition and adherence. Non-limiting examples of α-2,3-sialyltransferase are an amino acid sequence selected from: SEQ ID NOs: 31-33 or a sequence with at least 80%, 85%, 90%, 95°i°, 98% or 99% homology thereto.

α-2,6-sialyltransferase is used in resialylation and restoration of sialic acids (SAs). A non-limiting example of α-2,6-sialyltransferase is an amino acid sequence of: SEQ ID NO: 34 or a sequence with at least 80%, 85%, 90%, 95%, 98% or 99% homology thereto.

Sialyltransferase (PmST) is an enzyme that transfer sialic acid to nascent oligosaccharide. A non-limiting example of sialyltransferase is an amino acid sequence of: SEQ 1D NO: 35 or a sequence with at least 80%, 85%, 90%, 95%, 98% or 99% homology thereto.

UDP-N-acetylglucosamine 2-epimerase (UDP-GlcNAc 2-epimerase) is an enzyme that catalyzes the first two steps of the cytosolic formation of CMP-N-acetylneuraminic acid from UDP-N-acetylglucosamine. Non-limiting examples of UDP-GlcNAc 2-epimerase are an amino acid sequence selected from: SEQ ID NOs: 36-40 or a sequence with at least 80%. 85%, 90%, 95%, 98% or 99% homology thereto.

Table 1 identifies exemplary heterologous HMO formation enzymes for LNT and LNnT production:

| SEQID | | Gene and | | |
|---|---|---|---|---|
| NO | Enzyme | Organism | References | Notes |
| SEQ ID | β-1,3-N- | IgtA from | 2014, F | Produces |
| NO: 17 | acetylglucosaminyltransferase | Neisseria | Baumgrtner, et | LNTII |
| | | meningitidis | al. | |
| | | MC58 | Chembiochem. | |
| | | | 2014 Sep | |
| | | | 5;15(13):1896- | |
| | | | 900 | |
| SEQ ID | β-1,3-N- | Neisseria | 93% to Seq. ID | Produces |
| NO: 18 | acetylglucosaminyltransferase | polysaccharea | NO 17 | LNTII |
| | | ATCC 43768 | | |
| SEQ ID | β-1,3-N- | Homo sapiens | 33% to Seq. ID | Produces |
| NO: 19 | acetylglucosaminyltransferase | (Human) | NO 17 | LNTII |
| SEQ ID | β-1,3-galactosyltransferase | wbgO from | 2014, | Produces |
| NO: 20 | | Escherichia | Baumgrtner F., | LNT |
| | | coli O55:H7 | et al. | |
| | | | Chembiochem | |
| | | | 2014 Sep | |
| | | | 5;15(13):1896- | |
| | | | 900 | |
| SEQ ID | β-1,3-galactosyltransferase | Homo sapiens | 67% to Seq. ID | Produces |
| NO: 21 | | (Human) | NO 20 | LNT |
| SEQ ID | β-1,3-galactosyltransferase | Salmonella | 65% to Seq. ID | Produces |
| NO: 22 | | enterica | NO 20 | LNT |
| SEQID | β-1,4-galactosyltransferase | IgtB from | Dong X., et. | Produces |
| NO: 23 | | Neisseria | Biotechnol | LNnT |
| | | meningitides | Biofuels. 2019 | |
| | | | Sep 9,12:212 | |
| SEQID | β-1,4-galactosyltransferase | Neisseria | 90% to Seq. ID | Produces |
| NO: 24 | | polysaccharea | NO 23 | LNnT |
| SEQ ID | β-1,4-galactosyltransferase | Homo sapiens | 26% to Seq. ID | Produces |
| NO: 25 | | (Human) | NO 23 | LNnT |
| SEQ ID | glycosyltransferase family 2 | Neisseria | Priem B. et al. | Produces |
| NO: 42 | protein (IgtA) | meningitidis | Glycobiology, | LNTII |
| | | | 2002, Apr; | |
| | | | 12(4): 235- | |
| | | | 240. | |

Table 2 identifies exemplary heterologous HMO formation enzymes for 3′-SL and 6′-SL production:

| | | Gene and | | |
|---|---|---|---|---|
| SEQ ID NO | Enzyme | Organism | References | Notes |
| SEQ ID NO: | NeuNAc Synthase | Campylobacter | Sundaram A K., | Produces |
| 26 | | jejuni | el al. Biochem J. | NeuNAc |
| | | | 2004 Oct | |
| | | | 1;383(Pt 1):83-9 | |
| SEQ ID NO: | NeuNAc Synthase | E. coli | Zhang et al., | Produces |
| 27 | | | Biotechnol | NeuNAc |
| | | | Bioeng 2018 | |
| | | | Sep;115(9):2217- | |
| | | | 2231 | |
| SEQ ID NO | NeuNAc Synthase | C. jejuni ATCC | Fierfort N, | Produces |
| 28 | | 43438 | Samain E. J | NeuNAc |
| | | | Biotechnol. 2008 | |
| | | | Apr 30; 134(3- | |
| | | | 4):261-5 | |
| SEQ ID NO: | CMP-NeuNAc | Neisseria | Priem P., et al. | Produces CMP- |
| 29 | Synthetase | meningitidis | Glycobiology. | NeuNAc |
| | | | 2002 | |
| | | | Apr; 12(4):235- | |
| | | | 40. | |
| SEQ ID NO: | CMP-NeuNAc | C. jejwii A TCC | Fierfort N, | Produces CMP- |
| 30 | Synthetase | 43438 | Samain E. J | NeuNAc |
| | | | Biotechnol. 2008 | |
| | | | Apr 30;134(3- | |
| | | | 4):261-5 | |
| SEQ ID NO: | α-2,3- | Neisseria | Priem P., et al | Produces 3′-SL |
| 31 | sialyl transferase | meningitidis | Glycobiology | |
| | | | 2002 | |
| | | | Apr;12(4):235- | |
| | | | 40. | |
| SEQ ID NO: | α-2,3- | Trypanosoma | Wilbrink M. H., | Produces 3′-SL |
| 32 | sialyltransferase | | et al., Appl | |
| | | | Environ | |
| | | | Microbiol 2014 | |
| | | | Oct;80(19):5984- | |
| | | | 91. | |
| SEQ ID NO: | α-2,3- | Neisseria | Fierfort N, | Produces 3′-SL |
| 33 | sialyltransferase | gonorrhoeae | Samai n E. J | |
| | | | Biotechnol. 2008 | |
| | | | Apr 30;134(3- | |
| | | | 4):261-5 | |
| SEQ ID NO: | α-2,6- | Photobacterium | Drouillard S., et | Produces 6′-SL |
| 34 | sialyltransferase | sp. JT-ISH-224 | al Carbohydr | |
| | | | Res. 2010 Jul | |
| | | | 2,345(10):1394- | |
| | | | 9. | |
| SEQ ID NO: | sialyltransferase | Pasteurella | Guo Y., et al. J | Produces |
| 35 | (PmST) | multocida | Biotechnol. 2014 | 3′-SL 6′-SL |
| SEQ ID NO: | UDP-GlcNAc2- | Synechocystis | Jan 20; 170:60-7. | simultaneously |
| 36 | epimerase | sp. PCC6803, | Ishi kawa and | Produces |
| | slr1975 | | Koizumi | ManNAc |
| | | | Carbohydr Res. | |
| | | | 2010 Dec | |
| | | | 10;345(18):2605- | |
| | | | 9. | |
| SEQ ID NO: | UDP-GlcNAC 2- | Escherichia coli | Vann W F, et al. | Produces |
| 37 | epimerase | O1:K1 | J Bacterial. 2004 | ManNAc |
| | (neuC) | | Feb;186(3):706- | |
| | | | 12. | |
| SEQ ID NO: | UDP-GlcNAc 2- | Acinetobacter | Tzu-Ping Ko., et. | Produces |
| 38 | epimerase | baumannii | J Biol Chem. | ManNAc |
| | (neuC) | | 2018 Jun 29; | |
| | | | 293(26): 10119- | |
| | | | 10127. | |
| SEQ ID NO: | UDP-GlcNAc 2- | Neisseria | Zhang L., et al. | Produces |
| 39 | epimerase | meningitidis | Carbohydr Res. | ManNAc |
| | (neuC) | | 2016 Jan,419:18- | |
| | | | 28 | |
| SEQ ID NO: | UDP-GlcNAc 2- | Streptococcus | Vann W F., et al, | Produces |
| 40 | epimerase | agalactiae | J Bacteriol. 2004 | ManNAc |
| | (neuC) | | Feb; 186(3):706- | |
| | | | 12. | |

| IgtA from Neisseria meningitidis MC58 | |
|---|---|
| >WP_002257440.1 glycosyltransferase family 2 protein | |
| [Neisseria meningitidis] | |
| SEQ. ID NO: 17: | |
| 1 | MPSEAFRPHR AYRENKLQPL VSVLICAYNV EKYEAQSLAA VVNQTWRNLD ILIVDDGSTD |
| 61 | GTLAIAQRFQ EQDGRIRILA QPRNSGLIFS LNIGLDELAK SGGGGEYIAR TDADDIAAFD |
| 121 | WTEKIVGEME KDRSTIAMGA WLEVLSEEKD GNRLARHHEH GKIWKKPTRH EDIADFFPFG |
| 181 | NPIHNNTMIM RRSVIDGGLR YNTERDWAED YQFWYDVSKL GRLAYYPEAL VKYRLHANGV |
| 241 | SSKYSIRQHE TAQGTOKTAR NDFLQSMGFK TRFDSLEYRQ IKAVAYELLE KHLPEEDFER |
| 301 | ARRFLYQCFK RTDTLPAGAW LDFAADGRMR RLFTLRQYFG ILHRLLKNR |
| 93% to SEQ. ID NO: 17: | |
| >EFH23419.1 lacto-N-neotetraose biosynthesis glycosyl | |
| transferase LgtA | |
| [Neisseria polysaceharea ATCC 43768] | |
| SEQ. ID NO: 18: | |
| 1 | MTCAYNVEKY EAQALDAVVG QTWRNLDTLI VDDGSTDGTL ALAKDFQKRD SRTKLLAQAQ |
| 61 | NSGLIPSLNI GLDELAKSGG GEYIARTDAD DIAAPDWIEK IVGEMEKDRS IIAMGAWLEV |
| 121 | LSEEKDGNRL ARHHEHGKIW KKPTRHEDIA AFFPFGNPIH NNTMIMRRSV IDGGLRYNTE |
| 181 | RDWAEDYQFW YDVSKLGRLA YYPEALVKYR LHANQVSSKH SIRQHEIAQG IQKTARNDFL |
| 241 | QSMGFKTRFD SLEYRQTKAA AYELLEKDLP EEDFERARRP LYQCFKRTDT PPAGAWLDEA |
| 301 | ADGPMRRLFT LRQYFSILHR LIRNRRQARS DSAGKEQEI |
| 33% to SEQ. ID NO: 17 (Human): | |
| >NP_001306004.1 N-acetyllactosaminide | |
| beta-1,3-N-acetylglucosaminyltransferase 2 | |
| [Homo sapiens] | |
| SEQ. ID NO: 19: | |
| 1 | MSVGRRRIKL LGILMMANVF IYFTMEVSKS SSQEKNGKGE VIIPKEKFWK ISTPPEAYWN |
| 61 | REQEKLNPQY NPTLSMLTNQ TCEAGRLSNT SHLNYCEPDL RVTSVVTGFN NLPDRFKDFL |
| 121 | LYLRCRNYSL LIDQPDKCAK KPFLLLATKS LTPHFARRQA IRESWCQESN AGNQTVVRVE |
| 181 | LLGQTPPEDN HPDLSDMLKP ESEKHQDILN WNYRDTPTNL SLKEVLFLRW VSTSCPDTEF |
| 241 | VFKGDDDVFV NTHHILNYLN SLSKTKAKDL FIGDVINNAG FHRDKRLKYY IPEVVYSGLY |
| 301 | PPYAGGGGFL YSGHLALRLY HITDQVHLYP IDDVYTGMCL QKLGLVPEKH KGFRTFDIEE |
| 361 | KNKNNICSYV DLMLVHSRKP QEMIDIWSQL QSAHL |
| wbgO (WbgO from Escherichia coli O55:H7) | |
| >WP_000582563.1 Putative glycosyltransferase WbgO | |
| OS = Escherichia coli O55:H7 | |
| SEQ. ID NO: 20: | |
| 1 | MIIDEAESAE STHPWVSVIL PVNKKNPFLD EAINSILSQT FSSFEIIIVA NCCTDDFYNE |
| 61 | LKHKVNDKIK LIRTNIAYLP YSLNKAIDLS NGEFTARMDS DDISHPDRFT KQVDFLKNNP |
| 121 | YVDVVOTNAI FTDDKGREIN KTKLPEENLD IVKNLPYKCC TVHPSVMFRK KVIASTGGYM |
| 181 | FSNYSEDYEL WNRLSLAKTK FQNLPEYLFY YRLHEGQSTA KKNLYMVMVN DLVIKMKCFF |
| 241 | LTGNINYLFG GIRTIASFIY CKYIK |
| 67% to SEQ. ID NO: 20: | |
| >NP_001009096.1 B3GT1_HUMAN Beta-1,3-galactosyl | |
| transferase 1 [Homo sapiens] | |
| SEQ. ID NO: 21: | |
| 1 | MASKVSCLTV LTVVCWASAL WYLSITRPTS SYTGSKPFSH LTVARKNFTE GNTRTRPINP |
| 61 | HSFEFLINEP NKCEKNTPFL VILISTTHRE FDARQAIRET WGDENNFKGI KIATLFLLGK |
| 131 | NADPVLNQMV EQESQIPHDI IVEDFIDSYH NTTLKTIMGM RWVATFCSKA KYVMKTDSDI |
| 181 | FVNMDNLIYK LLKPSTKPRR RYFTGYVING GPIRDVRSKW YMPRDLYPDS NYPPFCSGTG |
| 241 | YIFSADVAEL IYKTSLHTRL LHLEDVYVGL CLRKLGIHPF QNSGFNHWKM ATSLCRYRRV |
| 301 | ITVHQISPEE MHRIWNDMSS KKHLRC |
| 65% to SEQ. ID NO: 20: | |
| >EBL7411458.1 glycosyl transferase | |
| [Salmonella enterica] | |
| SEQ. ID NO: 22: | |
| 1 | MLPVNKFNPY LDRATHSILS QSYPSIELII IANNCTNDFE DALKKRECET IKVLRTNTAY |
| 61 | LPYCLNKGLD LCNGDFVARM DSDDISHPER LDRQVDFLIN NPDIDVVGTN AVYIDEDDVE |
| 121 | LEKSNLPENN NAIKKMLPYK CCLVHPSVME RENWVTSSGG YMFANYSEDY ELWNRLAVEG |
| 181 | RTFYNLSEYL LYYRLHNNQS TSKNNLEMVM VNDVAIKVKY FLLTKKVSYL LGIIRTVFSV |
| 241 | FYCKYIK |
| IgfB from Neisseria meningitidis | |
| >WP_0022258244 LGTB NEIMB Lacto-N-neotetraose | |
| biosynthesis glycosyl transferase LgtB | |
| [Neisseria meningitidis serogroup B (strain MC58)] | |
| SEQ. ID NO: 23: | |
| 1 | MQNAVISLAS AAERRAHIAD TFGRHGIPFQ FFDALMPSER LEQAMAELVP GLSAHPYLSG |
| 61 | VEKACFMSHA VLWKQALDEG LPYITVFEDD VLLGEGAEKF LAEDAWLQER FDPDTAFIVR |
| 121 | LETMFMHVLT SPSGVADYCG RAFPLLESER WGTAGYIISR KAMRFFLDRF AALPPEGLHP |
| 181 | VDLMMFSDFF DREGMPVCQL NPALCAQELH YAKPRDQNSA LGSLIEHDRL LNRKQQRRDS |
| 241 | PANTFKHRLI RALTRISRER EKRRQRREQF IVPFQ |
| 90% to SEQ. ID NO: 23: | |
| >WP_003751005.1 glycosyltransferase family | |
| 25 protein. [Neisseria polysaccharea] | |
| SEQ. ID NO: 24: | |
| 1 | MQNHVISLSS AAERRAHZAA TFGAHGIPFG FFDALMPSER LEQAMAELVL GLSAHPYLSG |
| 61 | VEKACFMSHA VLWKQALDEG TFYTAVFEDD VLLSEGAEQF LAEDAWLQER FDPDSAETVR |
| 121 | LBTMFMHVLT SFSGVADYCG RAFPLLESEE WGTAGYITSK KAMRFFLDRF AALPSEGLHP |
| 181 | IDWMMFGNPD DRERMPVFQL NPALCAQELH YAKFHDQNSA LGSLTEHDRG LNRKQQRRDS |
| 241 | PANTFKHRLI RALTKISRER EKRPQRREQL IGKIIVPFQ |
| 26% to SEQ. ID NO: 23: | |
| >NP_004766.2 beta-1,4-galactosyltransferase 6 | |
| isoform 1 [Homo sapiens] | |
| SEQ. ID NO: 25: | |
| 1 | MSVLRRMMRV SNRSLLAFIF FFSLBSSCLY FIYVAPGIAN TYLFMVQARG IMLRENVETT |
| 61 | GRMTRLYTNK NSTLNGTDYP EGNNSSDYLV QTTTYLPENF TYSPYLPCPE KLPYMRGFLN |
| 121 | VNVSEVSFDE THQLFSKDLD IEPGGHWRPK DCKPRWKVAV LLPFRNRHEE LPIFFLHLIP |
| 181 | MLQKQRLEFA FYVIEQTGTQ PFNRAMLFNV GFKEAMKDSV WDOVIEHDVD HLPENDRNYY |
| 241 | GCGEMPRREA AKLDKYMYIL PYKEFFGGVS GLTVEQFRKI NGFPNAFWGW GGEDDDLWNR |
| 301 | VHYAGYNVTR PEGDLGKYKS IPHHHRGEVQ FLGRYKLLRY SKERQYIDGL NNLIYRPKIL |
| 361 | VDRLYTNISV NLMPELAPIE DY |
| >WP_002858213.1 Sialic acid synthase | |
| [Campylobacter jejuni] | |
| SEQ. ID NO: 26 | |
| 1 | MQIKIDKLTI SQKNPLIIPE IGINHNGSLE LAKLMVDAAK RAGAKTIKRQ TRIVEDEMSQ |
| 61 | EAKNVIPGNA NISIYEIMEQ CALNYKDELA LKEYVEKQGL VXLSTPFBRA AANRLEDMGV |
| 121 | SAYKTGSGEC NNYPLLKHIA QFKKPMIIST GMNSTESIKP TVKILRDYEI PFVLLHTTNL |
| 181 | YPTPSELVRL QAMLELYKEF NCLYGLSDHT TNNLACIGAI ALGASVLERE FTDTMDRKGP |
| 241 | DIVCSMDEST LKDLINQTQE MVLLRGDNNK NPLKEEQVTI DFAFASVVSI KDIKKGEILS |
| 301 | MDNIWVKRPS KGGISAKDFE AILGKRAKKD IKNNIQLTWD DFE |
| >WP_000066000.1 Sialic acid synthase NeuB | |
| [Escherichia coli O1:K1] | |
| SEQ. ID NO: 27 | |
| 1 | MSNTYIVAEI GCNHNGSVDI AREMTLKAKE AGVNAVKEQT FKADKLISAI APKAEYQIKN |
| 61 | TGELESQLEM TKKLEMKNNY YLHIMEYAVS LNLDVPSTPE DEDSIDFLAS LKQKIWKIPS |
| 121 | GELLNLPYLE KIAKLPIPDK KIIISTGMAT IDEIKQSVSI FINNKVPVGN ITILHCNTEY |
| 181 | PTPFEDVNLN AINDLKKHFP KNNIGFSDBS SGFYAAIAAV PYGITFIEKE FTLDRSMSGP |
| 241 | DHLASIEPDE LKHLCIGVRC VERSLGSNSK VVTASERKNK IVARRSIIAK TEIKKGEVFS |
| 301 | EKNTTTKRPG NGISPMEWYN LLGKIAEQDE IPDELIIHSE FKNQGE |
| >WP_002874241.1 N-acetylneuraminate synthase | |
| [Campylobacter jejuni] | |
| SEQ. ID NO: 28 | |
| 1 | MKEIKIQNII ISEEKAPLVV PEIGINHNGS LELARIMVDA AFSAGAKIIK HQTHIVEDEM |
| 61 | SKAAKKVIPG NAKISTYEIM QKCALDYKDE LALKEYTEKL GLVYLSTPFS RAGANRLEDM |
| 121 | GVSAFKTGSG ECNNYPLIKH LAAFKKEMIV STGMNSIESI KPTVKILLDN EIPFVLMHTT |
| 181 | NLYPTPHNTN RLNAMLELKK EPSCMVGLBD HTTDNLACLG AVVLGACVLE RHFTDSMHRS |
| 241 | GPDIVCSMDT KALKELTIQS EQMAIIRGNN ESKKAARQEQ VTLDFAFASV VBTKDLKRGE |
| 301 | VESMDNIWVK RPGLGGISAA EFENTLGKKA LRDTENDAQL SYEDFA |
| >AAA20476.1 CMP-NeuNAc synthetase | |
| [Neisseria meningitidis] | |
| SEQ. ID NO: 29 | |
| 1 | MEKGNIAVIL ARQNSKGLPL KNLRRMNGIS LLGHTINAAI SSKCFDRIIV STDGGLIAEE |
| 61 | AKNFGVEWVL RPAELASDTA SSISGVIHAL ETIGSNSGTV TLLQPTSPLR TGAHIREAFS |
| 121 | LFDEKIKGSV VSACPMEHHP LKTLLQINNG EYAPMRRLSD LEQPRQQLPQ APRENGAIYI |
| 181 | NDTASLIANN CFFIAPTKLY IMSHQDSIDI DTELDLQQAE NILNHKES |
| >WP_002869017.1 Acylneuraminate cytidylyltransferase | |
| [Campylobacter jejuni] | |
| SEQ. ID NO: 30 | |
| 1 | MSLAIIPARG GSKGTKNKNL VLLNNKPLIY YTIEAALNAK SISKVVVSSD SDEILNYAKS |
| 61 | QNVDILRRPI SLAQDDTTSD KVLLHALRFY KDYEDVVFLQ PTSPLRTNIH IDRAFNLYKN |
| 121 | SNANALISVS ECDNKILKAE VCNDYGDLAG ICNDEYPEMP RQKLPKTYMS NGAIYILKIK |
| 181 | EFLNNPSFLC NKTKHFLMDE SSSLDIDCLE DLKKVEQIWK K |
| >AJC62560.1 CMP-N-acetylneuraminate-beta- | |
| galactosamide-alpha-2 3-sialyltransferase | |
| [Neisseria meningitidis LNP21362] | |
| SEQ. ID NO: 31 | |
| 1 | MFNLSEWSFR DMGLKKACLT VLCLIVFCFG IFYTFDRVNQ GERNAVSLLK EKLFNEEGEP |
| 61 | VNLIFCYYIL QMKVAERINA QHPGERFYVV LMSENRNEKY DYYFNQIKDK AERAYFFHLP |
| 121 | YGLNKSFNFI PTMAELKVRS MLLPKVKRIY LASLERVSIA AFLSTYPDAE IKTFDDGTGN |
| 131 | LIQSSSYLGD BFSVNGTIKR NFARMMIGDW SIARTRNASD EHYTIFRGLE NIMDDGRRKM |
| 241 | TYLPLFDASE LKTGDETGGT VRTLLGSPDK EMKBISEKAA KNFKIQYVAP HPRQTYGLSG |
| 301 | VTTLNSPYVI EDYILREIKK NPHTRYEIYT PFSGAALTMK DFPNVHVYAL KPASLPEDYW |
| 361 | LKPVYALFTQ SGIPTLTFDD KN |
| >AAA99444.1 trans-sialidase [Trypanosoma cruzi] | |
| SEQ. ID NO: 32 | |
| 1 | MLAPGSSRVE LPKRQSSKVP FEKDGKVTER VVHSFRLPAL VNVDSVMVAI ADARYETSND |
| 61 | NSLIDTVVKY SVDDGETWET QTAIKNSRAS SVSRVVDPTV TVKGNKLYVI VGSYNSSRSY |
| 121 | WTSHGDARDW NTLLAVGEVT KSTAGGKITA SIKWGSPVSL KEFFPAEMEG MHTNQFLGGA |
| 181 | GVAIVASNGN LNYPVQVTNE RKQVFSKIFY SEDDGKTWKE CRGRSDFGCS EPVALEWEGR |
| 241 | LIINTRVDYR RRLVYESSDM GNSWVEAVGT LSRVWGPSPK SNQPGSQSSF TAVTTEGMRV |
| 301 | MLETHPLNFK GRWLRDRLNL WLTONQRIYN VGQVSIGDEN SAYSSVLYKD DKLYCLHEIN |
| 361 | SNEVYSLVFA RLVGELRIIK SVLQSWKNWD SHLSSICTPA DPAASSSERG CGPAVTTVGL |
| 421 | VGFLSHSATK TEWEDAYRCV NASMANAERV PNGLKFAGVG GGALWEVSQQ GQNQRYRFAN |
| 481 | HAFTVVASVT THEVPSVASP LLGASLDSSG GKKLLGLSYD EKHQWQPIYG STPVTPTGSW |
| 541 | ETGKRYHVVL TMANKIGSVY TDGEFLEGSG QTVVPDERTP DISHFYVGGY KRSDMPTISH |
| 601 | VTVNNVLLYN RQLNAEEIRT LFLSQDLIGT EAHMDSSSDT SA |
| >sp\|P72074 alpha-2,3-sialyltransferase | |
| [Neisseria gonorrhoeae] | |
| SEQ. ID NO: 33 | |
| 1 | MGLKKVCLTV LCLIVFCFGI FYTFDRVNQC ERNAVSLLRD KLFNEEGKPV NLIFCYTILQ |
| 61 | MKVAERIMAQ HPGERFYVVL MSENRNEKYD YYFNOIKDKA ERAYFFYLPY GLNKSFNFIP |
| 121 | TMAELKVKSM LLPKVKRIYL ASLEKVSIAA FLSTYPDAEI KTFDDGTNNL IRESSYLGGE |
| 181 | EAVNGAIKRN EARMMVGDWS IAKTPNASDE HYTIFKGLKN IMDDGRRKMT YLPLFDASEL |
| 241 | KAGDETGGTV RTLLGSPDKE MKBISEKAAK NFNTQYVAPH FRQTYGLSGV TALNSPYVIE |
| 301 | DYTLRETKKN PHTRYEIYTF FSGAALTMKD FPNVHVYALK PABLPEDYW1 KPVYALFRQA |
| 361 | DIPILTFDDK N |
| >pdb\|2Z4T\|A Beta-galactoside alpha-2,6-sialyltransferase | |
| [Photobacterium sp. JT-ISH-224] | |
| SEQ. ID NO: 34 | |
| 1 | MKNFLLLTLI LLTACNNSEE NTQSIIKNDI NKTIIDEEYV NLEPINQSNI SPTKHSWVQT |
| 61 | CGTQQLLTEQ NKESISLSVV APRLDDDEEX CFDFNGVSNK GEKYITKVTL NVVAPSLEVY |
| 121 | VDHASLPTLQ QLMDIIKSEE ENPTAQRYIA WGRIVPTDEQ MKELNITSFA LINNHTPADL |
| 181 | VQETVKQAQT KHRLNVKLSS NTAHSFDNLV PILKELNSFN NVTVTNTDLY DDGSAEYVNL |
| 241 | YNWRDTLNKT DNLKTGKDYL EDVINGINED TSNTGTSSVY NWQKLYPANY HFLRKDYLTL |
| 301 | EPSLHELRDY IGDSLKOMQW DGFKKFNSKQ QELFLSIVNF DKQKLQNEYN SSNLPNFVFT |
| 361 | GTTVWAGNHE REYYAKQQIN VINNAINESS PHYLGNSYDL FFKGHPGGGI INTLIMONYP |
| 421 | SMVDIPSKIS FEVLMMTDML PDAVAGIASS LYFTIPAEKI KFTVFTSTET TTDRETALRS |
| 481 | PLVQVMTRLG TVKEENVLEW ADLPNCETGV CIAV |
| >AAY89061.1 alpha-2,3/2,6-sialyltransferase/sialidase | |
| [Pasteurella multocida] | |
| SEQ. ID NO: 35 | |
| 1 | MKNRRLNFKL FFLIIFSLFS TLSWSKTITL YLDPASLEAL NQLMDFTQNN EDKTHPRIFG |
| 61 | LSRFKIPDNI TTQYONIHFV ELKDNRPTEA LFTILDQYPG NIELNIHLNT AHSVQLIRPI |
| 121 | LAYRFKHLDR VSTQQLNLYD DGSMEYVDLE KEENKDISAE IKQAEKQLSE YLLTGKIKFD |
| 181 | NPTIARYVSQ SAFPVKYHFL STDYFEKAEE LQPLKEYLAE NYQKMDWTAY QQLTPEQQAF |
| 241 | YLTLVGFNDE VKQSLEVQQA KFIFTGTTTW EGNTDVREYY AQQQLNLLNE FTQAEGDLFI |
| 301 | GDHYKIYFRG HPRGGEINDY ILNNAKNTTN IPANISFEVL MMTGLLPDKV GGVASSLYFS |
| 361 | LPKEKTSHII FTSNKQVKSK EDALNNPYVK VMRRLGIIDE SQVIFWDSLK QL |
| >WP_010872833.1 AGE family epimerase/isomerase | |
| [Synechocystis sp. (strain PCC 6803/Kazusa)] | |
| SEQ. ID NO: 36 | |
| 1 | MIANRRQELA QQYYQALHQD VLPFWEKYSL DBQGGGYFTC LDRKGQVPDT DKFIWLONRQ |
| 61 | VWQFAVFYNB LEPKPQWLEI ARHGADELAR HGRDQDGNWY PALDQEGKFL BQPYNVFSDC |
| 121 | FAAMAFSQYA LASGAQEAKA TALQAYNNVL RRQENPKGQY EKSYPGTRPL KSLAVPMILA |
| 181 | NLTDEMEWLL PPTTVEEVLA QTVREVMTDF LDPEIGLMRE AVTPTGEFVD SFEGRLLNPG |
| 241 | RGIEAMWEMM DIAQRSGDRQ LQEQAIAVVL NTLEYAWDEE FGGIFYFLDR QGHPPQQLEW |
| 301 | DQKLWWVHLE PLVALAKGHQ ATGQEKCWQW FERVHDYAWS BFADPEYGEW FGYLNRRGEV |
| 361 | LLNLKGGKWK GCFHVPRALN LCAETLQLPV S |
| >WP_000723250.1 UDP-N-acetylglucosamine 2-epimerase | |
| (hydrolyzing) [Escherichia coli O1:K1] | |
| SEQ. ID NO: 37 | |
| 1 | MKKTLYVTGS RAEYGTVRRL LTMLRETPEI QLDLAVTGMH CDNAYGNTIH IIEQDNFNII |
| 61 | KVVDINTNTT SRTHTLRSMS VCLNSFGDPF SNNTYDAVMV LGDRYEIFSV AIAASMHNIP |
| 121 | LIHIHGGEKT LANYDEFTRH STTKMSKLHL TSTEEYKKRV TQLGEKPGSV FNIGSLGAEN |
| 181 | ALSLHLPNKQ ELELKYGSLL KRYFVVVEHP ETLSTQSVND QIDELLSATS FFKNTHDFIF |
| 241 | IGSNADTGSD IIQRRVKYFC KEYXFRYLIS IRSEDYLAMI KYSCGLIGNS SSGLIEVPSL |
| 301 | KVATINIGDR QKGRVRGASV IDVFVEKNAI VRGINISQDE KFISVVOSSS NPYFKENALI |
| 361 | NAVRIIKDFI KSKNKDYKDF YDTPECTTSY D |
| >WP_005281413.1 UDP-N-acetylglucosamine 2-epimerase | |
| (hydrolyzing) [Acinetobacter baumanmi] | |
| SEQ. ID NO: 38 | |
| 1 | MKKIAVFTGT RAEYGLLYWL MKDIQQDPEL ELQILATAMH ISPEHGETWK TIVQDGFEIT |
| 61 | ESVEMLLSSD PSSAVVKSMG VGLLGFADAL KBMQPNTLVV LGDRFEALAV TQAALIMQVP |
| 121 | VAALHGGEIT EGAYDESIRR AITKMSNIHE AAAEEYKKRI IQLGEQPERV FNVGALGLDH |
| 181 | IQRTTFKNIA BLSELYDFDF SKPYFLITYH PETNLLEENV SPLFDALKQI KDVNEVFSYP |
| 241 | NADNGNTNTV KAMLDLKAQL PDRVLLVKSF GIQNYLSVLK NALAMVGNSS SGLSEAPALQ |
| 301 | VPTVNIGDRQ KGRLRCESIL DVKLDENEIL BALQKAINFP DDQLSGVVPP LGLGNTSQKI |
| 361 | IELIKTTDFR KKAPFYDL |
| >AAA20475.1 SiaA [Neisseria meningitidis] | |
| SEQ. ID NO: 39 | |
| 1 | MKRILCITGT RADFGKLKPL LAYIENHPDL BLHLIVTGMH MMKTYGRTYK EVTRENYOHT |
| 61 | YLFSNQTQGE PMGAVLGNTI TFTSRLSDEI EPDMVMIHGD RLEALAGAAV GALSSRLVCH |
| 121 | TBGGELSGTV DDSIRHSISK LSHIHLVANE QRVTRLVGMG EKRKHIHIIG SPDLDVMASS |
| 181 | TLPSLEEVKE YYGLPYENYG ISMFRPVTTE AHLMPQYAAQ YFKALELSGQ NTTSIYENND |
| 241 | TGTESILQEL LKYQSDKFIA FPSIRFEYFL VLLKHAKEMV GNSSAGTREA PLYGVPSIDV |
| 301 | GTRQNNRHMG KSIIHTDYET ENTFDAIQQA CSLGKFEADD TFNGGDTRTS TEREAEVINN |
| 361 | PETWNVSAQK RFTDLNL |
| >AADS3075.1 NeuC [Streptococcus agafactiae COH1] | |
| SEQ. ID NO: 40 | |
| 1 | MKKTCIVTGS RAEYGIMKPL TQRLSKDKEV NLQTIATAMH LEEKYGYTYR QIEEDGPDIA |
| 61 | YKVPLHLYDT DRRTVSTAMA HLQLGLTKIF DKEDYDLVII LGDRYEMLPV VNVALIYNVP |
| 121 | VCHLHGGETS LGNFDEYIRR AITKMSHLHL VSTEDFPQRV IQMGEQPQFV INTGALGVEN |
| 181 | ALSIPSLTKE AIEKQLGIVL EESYFWVLYH PVTFEQGKSA GEQMKAVLSA LSKFGVQCLE |
| 241 | IGSNSDTGSD DIAKAINTYL INHENSYCFA SLSTQLYHSL IRHSLGLIGE SSSGLIEVPS |
| 301 | LMKPTLNIGD RQKGRLHGES VVSVPVETPS VLEGLSKLNE VTNFDNPYYK ENASSIAYEA |
| 361 | IKLYLKDEPS TSQPFYDLKE NNLK |
| >WP_033911588.1 glycosyltransferase family 2 protein | |
| [Neisseria meningitidis] | |
| SEQ ID NO 42 | |
| 1 | MQPLVSVLTG AYNVEKYEAQ SLAAVVNQTW RNLDILIVDD GSTDGTLAIA QRFQEQDGRI |
| 61 | RTLAQPRNSG LIPSLNIGLD ELAKSGGGGE YIARTOADDT AAPDWIEKTV GEMEKDRSII |
| 121 | AMGAWLEVLS EEKDGNRLAR HHEHGKIWKK PTRHEDIADP FPPGNETHNN TMIMRRSVID |
| 181 | GGLRYNTERD WAEDYQFWYD VSKLGRLAYY PEALVKYRIH ANQVSSEYSI RQKEIAQGIQ |
| 241 | KTARNDFLQS MGFKTRFDSL EYRQTKAVAY ELLEKHLPEE DFERARRFLY QCFKRTDTLP |
| 301 | AGAWLDFAAD GRMRRLFTLR QYFGILHRLE KNR |

Genetically engineered microorganisms for use in combination with the HMO transporters (e.g., CDT-1 and variants) and with the methods can include other pathway enzymes. For example, for the production and export of LNnT or LNT, enzymes such as disclose in any of CN111534503, US2004175807, US2002142425, US2013030040, WO12168495, US2017204443 can be combined with CDT-1 or a variant of CD-1 to achieve export of LNnT or LNT. For example, for the production and export of 3′-SL or 6′SL, enzymes such as disclosed in any of US2005260718, US2017175155, CN106190938, CN111394292, CN101525627, US2008145899, US2009186377, WO19228993, US2020332331, US2008199942, US2018163185. US2005260729, US2005260729, KR20150051206, U.S. Pat. No. 9,637,768 can be combined with CDT-1 or a variant of CD-1 to achieve export of 3′-SL or 6′SL.

III. Production of HMOs in Microorganisms

HMOs are generally comprised of monosaccharides linked together, and typically with a lactose molecule at one end. Generally, the production of HMOs in microbes requires the presence of a starting monomer and one or more heterologous enzymes introduced into the microorganism. In some aspects, the monomer is a monosaccharide. In some aspects, the monomer is glucose, galactose, N-acetylglucosamine, fucose, and/or N-acetylneuraminic acid.

In one aspect, an engineered microorganism capable of producing a human milk oligosaccharide (HMO) is provided. Numerous embodiments are further provided that can be applied to any aspect of the present invention described herein. For example, in some embodiments, the microorganism comprises a first heterologous gene encoding an HMO formation enzyme. In some embodiments, the microorganism further comprises a second heterologous gene encoding a transporter, where the transporter facilitates the export of the produced HMO from the cell. In some embodiments, the HMO is an Lacto-N-Triose II (LNTII)-derived HMO or a sialylated HMO. In some embodiments, the HMO is a LNTII-derived HMO selected from lacto-N-neotetraose (LNnT) or lacto-N-tetraose (LNT). In some embodiments, the HMO is a sialylated HMO selected from 3′-sialyllactose (3′-SL) or 6′-sialyllactose (6′-SL).

In some embodiments, an engineered microorganism expressing one or more heterologous sequences, such as for an HMO formation enzyme and/or a transporter, includes regulatory sequences for such expression. In some embodiments, the endogenous promoter of a gene, such as that encoding an HMO formation enzyme and/or a transporter, is replaced with an exogenous promoter that induces the expression at a higher level than the endogenous promoter. In certain embodiments, the exogenous promoter is specific for the microorganism in which the exogenous promoter replaces the endogenous promoter. For example, a yeast specific exogenous promoter can be used if the microorganism being modified is a yeast. The exogenous promoter can be a constitutive promoter or inducible promoter.

Non-limiting examples of constitutive yeast specific promoters include: pCYC1, pADH1, pSTE5, pADH1, pCYC100 minimal, pCYC70 minimal, pCYC43 minimal, pCYC28 minimal, pCYC16, pPGK1, pCYC, pGPD or pTDH3. Additional examples of constitutive promoters from yeast and examples of constitutive promoters from microorganisms other than yeast are known to a skilled artisan and such embodiments are within the purview of the invention.

Non-limiting examples of inducible yeast specific promoters include: pGAL1, pMFA1, pMFA2, pSTE3, pURA3, pFIG1, pENO2, pDLD, pJEN1, pmCYC, and pSTE2. Additional examples of inducible promoters from yeast and examples of inducible promoters from microorganisms other than yeast are known to a skilled artisan and such embodiments are within the purview of the invention.

Microorganisms used to produce the genetically modified microorganisms described herein may be selected from Saccharomyces spp., such as S. cerevisiae, S. pastorianus, S. beticus, S. fermentati, S. paradoxus, S. uvarum and S. bayanus; Schizosaccharomyces spp., such as S pombe, S. japonicus, S. octosporus and S. cryophilus; Torulaspora spp. such as T. delbrueckii; Kluyveromyces spp. such as K. marxianus; Pichia spp. such as P. stipitis, P. pastoris or P. angusta, Zygosaccharomyces spp. such as Z. bailii; Brettanomyces spp. such as B. intermedius, B. bruxellensis, B. anomalus, B. custersianus, B. naardenensis, B. nanus; Dekkera spp., such as D. bruxellensis and D. anomala; Metschmkowia spp.; Issatchenkia spp. such as Lorientalis, Kloeckera spp. such as K. apiculata; Aureobasidium spp. such as A. pullulans; Torulaspora spp., Torulaspora delbrueckii, Zygosaccharomyces spp., Zygosaccharomyces bailiff, Brettanomyces spp., Brettanomyces intermedius, Brettanomyces bruxellensis, Brettanomyces anomalus, Brettanomyces custersianus; Brettanomyces naardenensis, Brettanomyces nanus, Dekkera spp., Dekkera bruxellensis, Dekkera anomala, Metschmkowia spp., Issatchenkia spp., Issatchenkia orientalis, Issatchenkia terricola, Kloeckera spp., Kloeckera apiculate, Aureobasidium spp., Aureobasidium pullulans, Rhodotorula spp., Rhodotorula glutinis, Rhodotorula cladiensis, Rhodosporidium spp., Rhodosporidium toruloides, Cryptococcus spp., Cryptococcus neoformans, Cryptococcus albidus, Yarrowia spp, Yarrowia lipolytica, Kuraishia spp, Kuraishia capsulata, Kuraishia molischiana, Komagataella spp., Komagataella phaffi, Komagataella pastoris, Hanseniaspora spp., Hanseniaspora guilliermondii, Hanseniaspora uvarum, Hasegawaea spp., Hasegawaea japonicas, Ascoidea spp., Ascoidea asiatica, Cephaloascus spp., Cephaloascus fragrans, Lipomyces spp., Lipomyces starkeyi, Kawasakia Spp., Kawasakia arxii, Zygozyma spp, Zygozyma oligophaga, Metschnikowia spp., Metschnikowia pulcherrima, Coccidiodes spp., Coccidiodes immitis, Neurospora discreta, Neurospora africana, Aspergillus spp., Aspergillus niger, Aspergillus nidulans, Aspergillus oryzae, Aspergillus fumigates, Mucor spp., Mucor circinelloides, Mucor racemosus, Rhizopus spp., Rhizopus oryzae, Rhizopus stolonifera, Umbelopsis spp., Umbelapsis isabelline, Mortierella spp, Mortierella alpine, Alternaria spp., Alternaria alternate, Botrytis spp., Botrytis cinereal, Fusarium spp., Fusarium graminarium, Geotrichum spp., Geotrichum candidum, Penicillium spp., Penicillium chrysogenum, Chaetomium spp., Chaetomium thermophila, Magnaporthe spp., Magnaporthe grisea, Emericella spp., Emericella discophora, Trichoderma spp., Trichoderma reesei, Talaromyces spp., Talaromyces emersonii, Sordaria spp., or Sordaria macrospora.

In specific embodiments, a microorganism, preferably, a fungus, such as a yeast, more preferably, a Saccharomyces spp., and even more preferably, S. cerevisiae is provided as the microorganism host. Yeast such as Saccharomyces spp. can be genetically engineered as described herein or using a multitude of available tools.

Other Ascomycetes fungi can also serve as suitable hosts. Many ascomycetes are useful industrial hosts for fermentation production. Exemplary genera include Trichoderma, Kluyveromyces, Yarrowia, Aspergillus, Schizosaccharomyces, Neurospora, Pichia (Hansenula) and Saccharomyces. Exemplary species include Pichia pastoris, Saccharomyces cerevisiae, Schizosaccharomyces pombe, Trichoderma reesei, Aspergillus stager, Aspergillus oryzae, Kluyveromyces lactis, Kluyveromyces marxianus, Neurospora crassa, Hansenula polymorpha, Yarrowia lipalytica, and Saccharomyces boulardii.

Cloning tools are widely known to those skilled in the art. See e.g., Cellulases and beyond: the first 70 years of the enzyme producer Trichoderma reesei, Robert H. Bischof, Microbial Cell Factories Volume 15, Article number: 106 (2016)), Development of a comprehensive set of tools for genome engineering in a cold- and thereto-tolerant Kluyveromyces marxianus yeast strain, Yumiko Nambu-Nishida, Scientific Reports volume 7, Article number: 8993 (2017); Engineering Kluyveromyces marxianus as a Robust Synthetic Biology Platform Host, Paul Cernak, mBio September 2018, 9 (5) e01410-18; DOI: 10.1128/mBio.01410-18; How a fungus shapes biotechnology: 100 years of Aspergillus niger research, Timothy C. Cairns, Fungal Biology and Biotechnology Volume 5, Article number: 13 (2018), GoldenPiCS: a Golden (late-derived modular cloning system for applied synthetic biology in the yeast Pichia pastoris, Roland Prielhofer, BMC Systems Biology Volume 11, Article number: 123 (2017)), Aiko Ozaki, “Metabolic engineering of Schizosaccharomyces pombe via CRISPR-Cas9 genome editing for lactic acid production from glucose and cellobiose,” Metabolic Engineering Communications Volume 5, December 2017, Pages 60-67, World 1 Microbiol Biotechnol. 2019; 35(1): 10. “Yarrowia lipolytica: a beneficious yeast in biotechnology as a rare opportunistic fungal pathogen: a minireview,” Bartlomiej Zieniuk (2014) “Functional Heterologous Protein Expression by Genetically Engineered Probiotic Yeast Saccharomyces boulardii.” PLOS ONE 9(11)); “Metabolic Engineering of Probiotic Saccharomyces boulardii,” Liu J-J, Kong II, 2016. Metabolic engineering of probiotic Saccharomyces boulardii. Appl Environ Microbiol 82:2280-2287; David Havlik. “Establishment of Neurospora crassa as a host for heterologous protein production using a human antibody fragment as a model product”. Microb Cell Fact. 2017; 16: 128; Ho, C. C. (April 1986). “Identity and characteristics of Neurospora intermedia responsible for oncom fermentation in Indonesia”. Food Microbiology. 3 (2): 115-132.

IV. Enhancement of Production and Export of HMOs

In some embodiments, the production and/or export of an HMO can be enhanced through genetic modification of an HMO-producing microorganism. For example, an HMO-producing microorganism can be modified by one or more of the following:

i) a genetic modification that increases the activity of PMA1 in the microorganism compared to PMA1 activity in the parental microorganism,

ii) a genetic modification that decreases the activity of SNF3 in the microorganism compared to SNF3 activity in the parental microorganism,

iii) a genetic modification that decreases the activity of RGT2 in the microorganism compared to RGT2 activity in the parental microorganism, and

iv) a genetic modification that decreases the activity of GPR1 in the microorganism compared to GPR1 activity in the parental microorganism.

In particular embodiments, i) the genetic modification that increases the activity of PMA1 is a genetic modification to plasma membrane ATPase gene (pma1), ii) the genetic modification that decreases the activity of SNF3 is a genetic modification to sucrose non-fermenting gene (snf3), iii) the genetic modification that decreases the activity of RGT2 is a genetic modification to glucose transport gene (rgt2), and iv) the genetic modification that decreases the activity of GPR1 is a genetic modification to G protein-coupled receptor 1 gene (gpr1). Examples of PMA1, SNF3, RGT2, and GPR1 are described in International Patent Application No. PCT/US2018/040351, the contents of which are incorporated herein by reference.

An example of PMA1 is provided by the sequence of SEQ ID NO: 5, which is PMA1 from Saccharomyces cerevisiae. Homologs of PMA1 from microorganisms other than S. cerevisiae, particularly, from yeast, can be used in the microorganisms and methods of the present disclosure. Non-limiting examples of the homologs of PMA1 useful in the instant disclosure are represented by Uniprot entries: A0A1U819G6, A0A1U8H4C1, A0A093V076, A0A1U8FCY1, Q08435, A0A1U7Y482, A0A1U8GLU7, P22180, A0A1U8G6C0, A0A1U8IAV5, A0A1U8FQ89, P09627, A0A199VNH3, P05030, P28877, A0A1U813U0, Q0EXL8, A0A1U813V7, P49380, Q07421, A0A1D8PJ01, P54211, P37367, P07038, Q0Q5F2, G8BGS3, A0A167F957, M5ENE2, A0A1B8GQT5, O74242, Q9GV97, Q6VAU4, A0A177AKN9, A0A1J6KB29, A0A2H9ZYJ6, A0A251UIM1, A0A251USM2, D2DVW3, MSBX73, Q6FXU5, A3LP36, G3ARI4, 9NSP9, A0A167C712, G2WE85, F2QNM0, A6ZUY5, C7GK65, A0A142GRJ4, W0T7K4, B3LDT4, A0A0H5BY16, A0A1B2J5T9, E7DB83, Q9UR20, F4NA03, Q96TH7, F4NA02,12G7P2, C4PGL3, F4NA00, F4N9Z6, Q7Z8B7, F4N9Z9, A0A1L4AAP4, O94195, A0A1D1YKT6, A0A0U1 YLR0, A0A0F8DBR8, A0A1C7N6N1, A0A2N6P2L5, A0A2C5WY03, O14437, T1VYW7, T1VY71, A1KAB0, C0QE12, K0NAG7, A0A0H3J1I1, A0A1Q9D817, A0A068MZP7, D1JED6, A0A2K8 WRE9, A0A1A8YFD7, A0A1A8YG89, I2G7P8, D9PN36, D1JI19, B61UJ9, B1XP54, H8W7G4, H6SL18, G8LCW3, L8AJP6, Q5ZFR6, A0A1D7QSR3, A0A1Q2TYG8, F4N054, A0A1Q9CTB2, A0A1Q9EJV5, A0A1D1XEE3, A0A0F7GAE0, D2DVW4, A0A0A9YX23, A0A1Q9ELW6. The Uniprot entries listed herein are incorporated by reference in their entireties.

Additional homologs of PMA1 are known in the art and such embodiments are within the purview of the present disclosure. For example, the homologs of PMA1 have at least 60%, 65%, 70%, 75%, 80%, 85%, 90%, 95%, or 99% sequence identity to SEQ ID NO: 5.

SEQ ID NO: 5:

| | |
|---|---|
| 1 | mtdtssasss ssassvsahq ptgekpakty ddaasesscd |
| | ddidalieel qsnhgvdded |
| 61 | sdndgpyaag earpvpeeyl qtdpsyglts devlkrrkky |
| | glnqmadeke slvvkfvmff |
| 121 | vgplqfviea aailaaglsd wydfgviagl lmlnagvgfy |
| | qefgagsivd elkktlanta |
| 181 | vvirdggive ipanevvpgd ilqledgtvi ptdgrivted |
| | cflqidqsaj tgeslavdkh |
| 241 | ygdqtfssst vkrgegfmvy tatgdntfvg raaalvnkaa |
| | ggqghftevl ngigiillvl |
| 301 | viatlllvwt acfyrtngiv rilrytlgit iigvpvglpa |
| | vvtttmavga aylakkqaiv |
| 361 | qklsaiesla gveilcsdkt gt1tknklsl hepytvegvs |
| | padimitacl sasrkkkgld |
| 421 | aidkaflksl kqypkakdal tkykvlethp fdpvskkvta |
| | vvespegeri vcvkgaplfv |
| 481 | 1ktveedhpl pedvhenyen kvaelasrgf xaigvarkrg |
| | aghweilgyt pcmdpprddt |
| 541 | aqtvsearhl gixvkmltge avgiaketcr qlglgtniyn |
| | aerlglgggg dmpgseladf |
| 601 | venadgiaev fpqhkyrwei ilqnrgylva mtgogvndap |
| | slkkadtgia vegatdaars |
| 661 | aadivflapg 1saiidalkt srqithrmya yvvyrialsl |
| | hleiflglwi aildnsldid |
| 721 | livfiaifad vatlaiaydr apyspkpvkw nlprlwgmsi |
| | ilgivlaigs witlttmflp |
| 781 | kggiiqntga mngimflqis ltenwlifit raagptwssi |
| | pswqlagavf avdiiatmft |
| 841 | lfgwwsenwt divtvvrvwi wsigifcvlg gfyyemstse |
| | afdrlmngkp mkekkstrsv |
| 961 | edfmaamgrv stgheket |

An example of SNF3 is provided by the sequence of SEQ ID NO: 6, which is SNF3 from S. cerevisiae. Homologs of SNF3 from microorganisms other than S. cerevisiae, particularly, from yeast, can be used in the microorganisms and methods of the present disclosure. Non-limiting examples of the homologs of SNF3 useful in the instant disclosure are represented by Uniprot entries: W0TFH8, Q6FNU3, A0A0W0CEX1, G2WBX2, A6ZXD8, J6EGX9, P10870, C7GV56, B3LH76, A0A0L8RL87, A0A0K3C9L0, M7WSX8, A0A1U8HEQ5, G5EBN9, A8X3G5, A3LZS0, G3AQ67, A0A1E4RGT4, A0A1B2J9B3, F2QP27, E3MDL0, A0A2C5X04S, G0NWE1, A0A0H5S3Z1, A0A2G5VCG9, A0A167ER19, A0A167DDU9, A0A167CY60, A0A167CEW8, A0A167ER43, A0A167F8X4, A0A1B8GC68, A0A177A9B0, E3EIS7, E3E8B6, A0A0A9Z0Q2. The Uniprot entries listed herein are incorporated by reference in their entireties.

Additional homologs of SNF3 are known in the art and such embodiments are within the purview of the present disclosure. For example, the homologs of SNF3 have at lost 60%, 65%, 70%, 75%, 80%, 85%, 90%, 95%, or 99% sequence identity to SEQ ID NO: 6.

SEQ ID NO: 6:

| | |
|---|---|
| 1 | mdpnanssse llrgekggfl dkalqrvkgi alrrnnsnkd |
| | httddttgsi rtptslqrqn |
| 61 | sdrqsamtsy ftddistidd nsilfseppg kqsmmmsicv |
| | gvfvavggfl fgydtglins |
| 121 | itsmnyvssh vapnhdsfta qqmsilvsfl slgtffgalt |
| | apfisdsygr kptiifstif |
| 181 | ifsignslqy gaggitlliv grvisgigig aisavvplyq |
| | aeathkslrg aiistyqwai |
| 241 | twgllvssav sggtharada ssyripiglq yywssflaig |
| | mi£lpespry yvlkakldea |
| 301 | akslsfirgv pvhdsgllee lveikatydy easfgssnfi |
| | dofissksrp kqtlrmftgi |
| 361 | alqafqqfsg infifyygvn ffnktgvsns ylvsfityav |
| | nvyfnvpglf fveffgrrky |
| 421 | lvvggvimti anfivaivgc slktvaaakv miaficlfia |
| | afsatwggvv wvisaelypl |
| 481 | gyrskotaic aaanwlvafi calitpyivd tgshtsslga |
| | kiffiwgsln amgvivvylt |
| 541 | vyetkgltle aidelyikss tgvvsphfnk direralkfq |
| | ydplqrledg kntfvakrni |
| 601 | fddetprndf rntisgeidh spagkevhsi pervdiptst |
| | eilespnkss gmtvpvspsl |
| 661 | gdvpipgtte paeirckyvd lgnglginty nrgppslssd |
| | ssedybedei ggpssqgdqs |
| 721 | nrstmndind ymarlihsts tasnttakfs gngstlryht |
| | asshsdttee dsnlmdlgng |
| 781 | lalnaynrgp psilmssde eanggetsdn intaqdlagm |
| | kermagfags yidkrgglep |
| 841 | etqsnilsts lsvmadtneh nneilhssee natnqpvnen |
| | ndlk |

An example of RGT2 is provided by the sequence of SEQ 113 NO: 7, which is RGT2 from S. cerevisiae. Homologs of RGT2 from organisms other than S. cerevisiae, particularly, from yeast, can be used in the microorganisms and methods of the present disclosure. Non-limiting examples of the homologs of RGT2 are represented by Uniprot entries: A0A0U1MAJ7, N4TG48, A0A1Q8RPY1, N4U710, A0A1L7SSQ2, A0A1L7VB15, A0A0C4E497, A0A1L7UAN6, A0A0J0CU17, A0A1L7VMA9, S0ED22, A0A1L7SD48, N1R8L8, A0A1L7V0N4, S3BYD3, E4UUU6, N4UPT5, N4U030, A0A0I9YK83, S0DJS4, A0A0U1LWH9, A0A0K6FSJ2, N1S6K7, A0A0J6F3E5, A0A1E4RS51, N4UTN2, A0A0G2E6D5, A0A1J9R914, A0A0F4GQX7, A0A1S9RLB9, A3MON3, J9PF54, A0A074WC52, A0A0K6GI66, N1QHS4, G2WXK0, B2VVL4, B2WDK7, A0A1J9S6A1, G4N0E9, L7JEU7, L71NA5, A0A0L1HE99, A0A0J8QL36, A0A0H5CKW2, A0A0J6Y4E2, W0VMG0, G2WQD8, A0A1C1WV61, A0A1S9RL33, C9SBA9, A0A0G2HY75, J3P244, N1QK04, A0A0N0NQR9, A0A1S7UJ19, G2XFE7, C9SWZ3, R8BUY9, M7SYH1, A0A1E1MIV2, A0A1E1LLK3, A0A1E1LJE1, L7J4Y3, L7I304, A0A1L7XU29, A0A136JCY3, A0A0J8RG81, A0A177DW33, A0A1L7X792, W9C8U1, B2VXL1, A0A0L1HMG8, A0A178DQW4, A0A167V6F7, A0A166WR60, A0A162KLT6, A0A1L7X3D1, G3JQX8, Q7S9U8, E9F7A6, A0A1S7HPX9, A0A0G2G564, A0A0W0D0B3, A6ZXI9, Q12300, C7GKZ0, G2WC23, A0A0H5CAT9, J4U3Y8, A0A0L8RL54. The Uniprot entries listed herein are incorporated by reference in their entireties.

Additional homologs of RGT2 are known in the art and such embodiments are within the purview of the present disclosure. For example, the homologs of RGT2 have at least 60%, 65%, 70%, 75%, 80%, 85%, 90%, 95%, or 99% sequence identity to SEQ U) NO: 7.

SEQ ID NO: 7:

| | |
|---|---|
| 1 | mndsqneirq teenshlnpg ndfgihqgae ctinhnnmph |
| | rnaytestnd teaksivmcd |
| 61 | dpnaygssyt nnepagdgai ettsillsqp lplrsnvmav |
| | lvgifvavgg flfgydtgli |
| 121 | nsitdmpyvk tyiapnbsyf ttsqiailvs flslgtffga |
| | llapyisdsy grkptimfst |
| 181 | avifsigusl qvasgglvll ivgrvisgig igiisavvpl |
| | yqaeaaqknl rgaiisayqw |
| 241 | aitigiivss avsqgthskn gpssyripig lqyvwssila |
| | vgmiflpaap ryyvikdeln |
| 301 | kaakslsflr glpiedprll eelveikaty dyeasfgpst |
| | lldcfktsen rpkailrift |
| 361 | giaiqafqqa sginfifyyg vnffnntgvd nsylvsfisy |
| | avnvafsipg mylvdrigrr |
| 421 | pyllaggvim aianlviaiv gvsegktvva skimiaficl |
| | fiaafsatwg gvvwvvsael |
| 481 | yplgvrskct aicaaanwlv nftcalitpy ivdvgshtss |
| | mgpkiffiwg glnvvavivv |
| 541 | yfavyetrgl tleeidelfr kapnsvissk wnkkirkrcl |
| | afpisqqiem ktniknagkl |
| 601 | dnnnspivqd dshniidvdg flenqiqsnd hmiaadkgsg |
| | slvniidtap ltstefkpve |
| 661 | bppvnyvdlg nglgintynr gppsiisdst defyeendss |
| | yynnnterng ansvntymaq |
| 721 | linsssttsn dtsfspshns nartssnwts dlaskhsqyt |
| | spq |

An example of GPR1 is provided by the sequence of SEQ II) NO: 8, which is GPR1 from S. cerevisiae. Homologs of GPR1 from microorganisms other than S. cerevisiae, particularly, from yeasts, can be used in the microorganisms and methods of the present disclosure. Non-limiting examples of the homologs of GPR1 are represented by Uniprot entries: A0A1S3ALF0, A0A0Q3MD2S, A0A146RBQ8, A0A0P5SHA9, A2ARI4, Q9BXB1, Q9Z2H4, F1MLX5, U3DQD9, I2CVT9, I0F144, K7D663, K7ASZ6, A0A1U7Q769, U3ESI5, T1E5B8, A0A0F7ZA01, J3RZW5, A0A094ZHC9, W6UL90, A0A0P6J7Q8, L5KYC3, B7P6N0, B0BLW3, A2AHQ2, A0A151N8W7, A0A146RCW3, A0A0X3NYB9, A0A0P5Y3G9, W5UAB2, A0A0P5IC44, A0A090XF51, A0A146NRV7, A0A0X3Q0R0, A0A0P61RD7, L9JFB7, A0A146YGG2, A0A146WG88, Q12361, B3LGT6, A0A0N8A6F9, P0DM44, W6JM29, A0A1A8LC80, A0A0N8A4D4, Q7Z7M1, A0A1S3G1Q8, A0A1U7QGH1, A6ZXT8, A0A1U8C0F6, D3ZJU9, A0A1S3KGL3, G5B385, L9KNY9, A0A1S3AQM3, A0A087UXX9, A0A0L8VW24, A0A0P6AR08, Q9HBX8, Q3UVD5, A0A1U7UEF2, A0A146XMF9, A0A146QTV1, A0A1S31D45, L5KTU9, A0A1A8ELT4, A0A0N7ZMX8, A0A0PSQ3T8, A0A1A8N9Z4, A0A1A8D807, A0A1A8CVG1, A0A1A8UMB1, A0A1A8JQ07, A0A1A8P7N2, A0A1A8H1,38, E7FE13, A0A1S3FZL3, A0A0P7WLQ9, H2KQN3, A0A1S3WJA9, A0A146PKA1, L5LLQ3, F1Q989, A0A0F8AKY3, A0A0P7VR95, A0A1U8C8I3, A0A034VIM3, A0A0N8BFD4, A0A146XMJ1, A0A0N8BDM1, A0A1A8KTJ1, A0A1A7X706, A0A0R4ITE3, A0A1U7S4H0, A0A1S3AQ94, A0A1U7UCP2, L8HMA8, A0A0Q3P3V6, A0A1A8CDG3, D6W7N2, A0A1E1XMY8, A0A1A8ACL5, A0A1S3WNV2, T0MHY5, A0A1S3G113, V8P2X5, A0A1S3KV51, A0A1S3G018, A0A1S3PUP5, A0A1U8C7X5, S9WP18, A0A1S3AQL8, A0A0N8ENF1, K7C1G0, A0A147BFY7, A0A1S3FZK9, A0A1U7TUH0, A0A1U8BX93, A0A091DKN5, A0A146W919, A0A147B2K7, A0A146XNL4, A0A091DTX9, A0A0Q3UQB0, A0A146WH37, E9QDD1, Q58Y75, A0A096MKI0, A0A1S3S901, Q14BH6, A0A1S3AQ42, A0A0P5SV49, A0A0P5P299, A0A0P5WCR4, K7CHT8, A0A1U7U0Q5, A0A1S3EXD4, A0A146Y6G0, A0A061HXQ0, A0A1S3AQ84, A0A1S2ZNQ3, A0A1U7UEE6, A0A1S3G013, A0A1U7QJG4, S7N7M1, A0A1S3G108, A0A1U8C8H8, and A0A1U8C7X0.

Additional homologs of GPR1 are known in the art and such embodiments are within the purview of the present disclosure. For example, the homologs of GPR1 have at least 60%, 65%, 70%, 75%, 80%, 85%, 90%, 95%, or 99% sequence identity to SEQ ID NO: 8.

SEQ ID 8:

| | |
|---|---|
| 1 | mitegfppnl nalkgsslle krvdsirqln tttvnallgl |
| | pgmtstftap qllqlriiai |
| 61 | tasavsliag clgmfflskm dkrrkvfrhd liafliicdf |
| | lkafilmiyp miilinnsvy |
| 121 | atpaffntlg wftafaiega dmaimifaih failifkpnw |
| | kwrnkrsgnm egglykkrsy |
| 181 | iwpitalvpa ilaslafiny nkinddsdtt iildnnnynf |
| | pdsprqggyk pwsawcylpp |
| 241 | kpywykivls wgpryfiiif ifavylsiyi fitseskrik |
| | aqigdfnhnv leeekekkkl |
| 301 | fglghwgkak wyfrsyfklp llhllrnlkn fftisfidpn |
| | eetddsgssn gtfnfgessn |
| 361 | eiptlfrktn tgsdenvsas ggvrlldyns akplemskya |
| | msegpalern npfdcendit |
| 421 | lnpselvskq kebkytfsve negldtrkss mlghqtfscq |
| | nslesplamy dnkndnsdit |
| 481 | snikekggii nnnsnndddd nnnnndndnd nnnsnnnnnn |
| | nnnnnnnnnn nnnnnnnnnn |
| 541 | nnnnsnnikn nvdnantnpa dniptlsnea ftpsqqfsge |
| | rvnnnadrce nssftnvagh |
| 601 | fqaqtykqmk krraqiqknl raifiyplsy igiwlfpiia |
| | dalqynheik hgptmsvtyi |
| 661 | dtcvrplscl vdvivylfke kpwnyswakt eskyliekyi |
| | lkgelgekei ikfchsnwgk |
| 721 | rgwyyrgkwk krkcwkystn plkrilwfve rffkqlfelk |
| | lhfsfydncd dfeywenyys |
| 781 | akdsndnart esdetktnss drslpsnsle iqamlnnita |
| | eevevplfwr iihhipmigg |
| 841 | idldelazil kirynndhfs lpglkfalnq nkshdkhqdv |
| | stnsmvkssf fssnivendd |
| 901 | ansieedknl rysdasasen ylvkptipgt tpdpiieaqn |
| | dndssdssgi dliaflrngp |
| 961 | l |

Substrates for Production of HMOs

In certain embodiments, the present disclosure provides microorganisms comprising one or more genetic modifications that provide for import and/or enhanced uptake of one or more substrates that can be used by the microorganism to make an HMO. For example, a microorganism can include:
i) a genetic modification that introduces a substrate transporter gene LAC12, or its analogues which increases the uptake of lactose and/or other substrate into the microorganism;
ii) a genetic modification that introduces a transporter which can both import a substrate, such as lactose and export a produced HMO, such as the wild type cellodextrin transporter gene cdt-1 or a variant of the cellodextrin transporter gene cdt-1 such as those described herein (for example, CDT-1 N209S F262Y, CDT-1 G91A, CDT-1 F213L, CDT-1 L256V, CDT-1 F335A, CDT-1 S411A, CDT-1 N209S F262W).

In certain embodiments, the present disclosure provides microorganisms where one or more endogenous transporters are upregulated or otherwise enhanced in activity (such as by upregulation of a transcription factor, which then increases the level of an endogenous transporter) to export. the HMO in addition to the CDT-1 or variant CDT-1. In some aspects, fermentation of the microorganism can include stress responses or other conditions that upregulate an endogenous transporter activity and such activity in combination with the activity of CDT-1 or a CDT-1 variant contributes to the export of the HMO produced by the microorganism. In some aspects, the stress response or condition is created or accentuated in larger scale fermentation conditions.

In certain embodiments, the present disclosure provide a genetic modification that introduces a transporter such as CDT-1 or a variant of CDT-1 (e.g., CDT-1 N209S F262Y, CDT-1 G91A, CDT-1 F213L, CDT-1 L256V, CDT-1 F335A, CDT-1 S411A, CDT-1 N209S F262W) and also a further genetic modification that increases production and/or export of the HMO such as one or more of increasing the activity of PMA1 or decreasing the activity of SNF3, RGT2 or GPR1 in the microorganism. In some aspects, the microorganism includes the introduction of CDT-1 or a variant of CDT-1, and genetic modifications that decrease the activity of SNF3 and RGT2.

Production, Separation and Isolation of HMOs

In some embodiments, the microorganisms described herein are capable of producing HMOs such as Lacto-N-Triose II (LNTII)-derived HMO or sialylated HMO. In some embodiments, the microorganisms are capable of converting lactose into Lacto-N-Triose II (LNTII)-derived HMO or sialylated LIMO. In particular embodiments, the microorganisms described herein have higher capacity, compared to the parental microorganisms, of converting lactose into Lacto-N-Triose II (LNTII)-derived HMO or sialylated HMO. In specific embodiments, the conversion of lactose into Lacto-N-Triose II (LNTII)-derived HMO or sialylated HMO occurs in the cytosol of the microorganisms.

In still another aspect, methods of producing products of interest by culturing the microorganisms described herein in appropriate media containing an appropriate oligosaccharide under appropriate conditions for an appropriate period of time and recovering an oligosaccharide from the culture media, is provided.

In certain embodiments, the disclosure provides methods of producing Lacto-N-Those II (LNTII)-derived HMO or sialylated HMO by culturing the microorganisms described herein in culture media containing lactose under appropriate conditions for an appropriate period of time and recovering Lacto-N-Triose II (LNTII)-derived HMO or sialylated HMO from the culture media.

In preferred embodiments, the microorganisms belong to Saccharomyces spp. In even more preferred embodiments, the microorganisms are S: cerevisiae.

In certain embodiments, the media contains about 10 g/L yeast extract, 20 g/L peptone, and about 40 g/L oligosaccharide, particularly, lactose or sucrose. In particular embodiments, the microorganisms, particularly, yeast, are grown at 30° C.

Additional culture media, conditions appropriate for culturing the microorganisms, and the methods of recovering the products of interest from the culture media are well known in the art and such embodiments are within the purview of the invention.

In certain aspects, the present disclosure provides methods for producing oligosaccharides by culturing the microorganisms described herein in the presence of appropriate oligosaccharides and recovering the products of interest. In some embodiments, an HMO is separated from the cells (microorganism) that produce the HMO. In some cases, an HMO can be further isolated from other constituents of the culture media (fermentation broth) in which the HMO-producing cells arc grown.

In some embodiment, an HMO is recovered from the fermentation broth (also referred to a culture medium). Many methods are available for separation of cells and/or cell debris and other broth constituents from the produced HMO.

For example, cell/debris separation can be achieved through centrifugation and/or filtration. The filtration can be microfiltration or ultrafiltration or a combination thereof. Separation of charged compounds can be achieved through ion exchange chromatography, nanofiltration, electrodialysis or combinations thereof. Ion exchange chromatography can be cation or anion exchange chromatography, and can be performed in normal mode or as simulated moving bed (SMB) chromatography. Other types of chromatography may be used to separate based upon size (size exclusion chromatography) or affinity towards a specific target molecule (affinity chromatography). For example, US 20.1.9/0119314 A1, GRAS applications GRN0005718 and GRN000749.

Drying or concentration steps can be achieved with evaporation, lyophilization, reverse osmosis, or spray drying. Crystallization can serve as a concentration and separation step and can be done with for example evaporative or temperature-based crystallization, or induced by modification of pH or increase in ionic strength. For example, US20170369920A1, WO2018164937A1.

Absorption techniques, such as adsorption using activated charcoal, can also be used as a separation step and in particular is useful for removal of color bodies or separation of oligosaccharides from monomers.

An HMO product can also be pasteurized, filtered, or otherwise sterilized for food quality purposes.

Exemplary Embodiments for Fermentation and Processing

In certain embodiments, microorganisms producing Lacto-N-Triose II (LNTII)-derived HMO or sialylated HMO described herein can be grown in fermentors to prepare larger volumes of HMOs. The fermentations can be operated in batch, fed-batch, feed and draw, or continuous mode. in some embodiments, dextrose (glucose) is used as the primary carbon and energy source for fermentation. In some embodiments, concentrated feeds are used to supply a carbon and energy source and/or lactose. In some embodiments, at least about 20 grams of glucose is used per titer of final working volume of the fermentor. In some aspects, at least about 50 g/L is used in the fermentation. In some aspects, at least about 100 g/L glucose is used, such as 150, 200, 250, 300, 350, 400 wt. In some embodiments, lactose is present or co-fed to the bioreactor at levels of 10-200 g/L final fermentor working volume, at a level of 25-150 g/L, or at 50-100 g/L. In some embodiments, the fed-batch fermentations are run with limiting concentrations of glucose or other nutrients. Non-continuous fermentations are run for 2-10 days or 4-6 days. Fermentor nominal sizes can be at least about 100 L, at least about 1000 L, greater than 10000 L, or at least about 100,000 L.

In some embodiments, the pH of the fermentation is kept constant throughout the culture. In some aspects, one or more of the pH setpoints is between about 3 to about 8, or about 4 to about 7, or about 4.5 to about 6.5 or about 5 to about 6. In some embodiments. the fermentation is controlled to one or more temperature setpoints. In some aspects, one or more of the temperature setpoints is between about 20° C. and about 40° C., or between about 25° C. and about 32° C., or is between about 29° C. and about 31° C. In some embodiments, media and or feed components used for cell culture are undefined (complex) ingredients, such as yeast extract. In some embodiments, defined media and/or feeds are used.

In certain embodiments, the Lacto-N-Triose II (LNTII)-derived HMO or sialylated HMO is present in the fermentation medium. Isolation of HMO product occurs through a series of downstream separations which can be run in continuous or batch mode. Unit operations include cell separation, concentration, desalting, decolorization, removal of impurities, sterilization, and drying (see e.g., Stanbury, P., Whitaker, A. & Hall, S. The recovery and purification of fermentation products. in Principles of Fermentation Technology 619-686 (2017)).

In some aspects, the cells of the microorganism are separated from the HMO by centrifugation. In some aspects, cross-flow (tangential flow) microfiltration clarifies the centrate and the HMO is in the permeate. In some aspects, polymeric or ceramic membranes of molecular weight cut-off values ranging from 501 kDa to 0.65 μm or 100 kDa to 0.45 μm clarify the centrate. In some embodiments, the molecular weight cutoff is 100 kDa. Membranes can be used in plate-and-frame, hollow-fiber, or spiral-wound configurations, in conjunction with diafiltration to improve product recovery in filtrate. Cross-flow microfiltration can be carded out with hollow-fiber or spiral wound configurations and diafiltration to improve product recovery in filtrate.

In some aspects, cross-flow nanofiltration largely desalts and concentrates the HMO and the HMO is in retentate. In some aspects, polymeric membranes with molecular weight cut-off values ranging from 200 to 1000 Da retain HMO product in the clarified centrate, with lower retention of monovalent and divalent salts. In some aspects, molecular weight cut off values range from 400 to 700 Da, for example the molecular weight cut-off is 500 Da. Non-limiting examples of nanofiltration membranes include Koch SR3D, Hydranautics Nitto Hydracore 70, Hydranautics Nitto DairyNF, Suez (GE) DK, Suez (GE) DL, Synder NFW, Synder NFG, Dow FilmTec NF270, Microdyn-Nadir TriSep XN45, Microdyn-Nadir TriSep TS40.

In some aspects, Cation/Anion Exchange: Further desalts and deodorizes the HMO and the HMO is in pass-through. In some aspects the HMO is subjected to 0.2 micron filtration, such as to remove bioburden (e.g., prior to drying). In some aspects the IMO is dried, by spray drying or by lyophilization. Non-limiting examples of anion exchange resins include Diaion HPA75, Diaion HPA2SL, Diaion PA308, and Diaion PA408. Non-limiting examples of cation exchange resins include Diaion PK216, Diaion PK208, and Diaion UBK10.

In some embodiments of the processing of the HMO, centrifugation can be replaced by using a cross-flow filtration step to fully clarify the broth, using lower fluxes as compared to a post-centrifugation filtration step, for example, a 100 kDa cross-flow filtration, optionally with diafiltration to improve product recovery.

In some embodiments of the processing of the HMO, one or both ion exchange steps can be replaced by desalting completely with nanofiltration. In some aspects, color bodies and/or impurities can be removed by activated charcoal or other adsorbents. Ethanol can be used to elute oligosaccharides from the charcoal column after highly water soluble components are rinsed away. Strongly hydrophobic impurities may require higher concentrations of alcohol to elute. In some aspects, the cross-flow filtration clarification step can be replaced by a filter press optionally using filter aid, and concentration of broth can optionally be done using evaporation or vacuum evaporation.

In some embodiments, electrodialysis can be used to remove salts in place of a nanofiltration or ion exchange step. In some embodiment crystallization can be used (for example methanol-based, ethanol-based, temperature-based, or evaporative) to remove organic impurities and/or salts. In some embodiments, pasteurization can replace the 0.2 micron filtration to reduce bioburden.

The methods herein for fermentation and downstream processing also find use in production of other HMOs, for example 2′-FL.

Other methods and components for processing and isolation of the HMOs herein can be employed, such as those disclosed in U.S. Ser. No. 10/377,787, EP3131912, EP3524067, EP3486326, WO201963757, EP3450443, WO201486373, WO2014086373, WO2015188834, U.S. Ser. No. 10/899,782, U.S. Pat. No. 9,896,470, EP3494806, as well as any of Karoly Agoston, et al. Kilogram scale chemical synthesis of 2′-fucosyllactose, Carbohydrate Research, Volume 476, 2019, Pages 71-77, Karina Altmann et al, Nanofiltration Enrichment of Milk Oligosaccharides (MOS) in Relation to Process Parameters, Food Bioprocess Technol (July 2019), Andreas Geisser, et al., Separation of lactose from human milk oligosaccharides with simulated moving bed chromatography, Journal of Chromatography A 1092 (2005) 17-23, Joshua L. Cohena, et al., Role of pH in the recovery of bovine milk oligosaccharides from colostrum whey permeate by nanofiltration, Int Dairy J. (2017 March), 66: 68-75, and Yaoming Wang, et al., Electrodialysis-Based Separation Technologies in the Food Industry, Chapter 10 in book: Separation of Functional Molecules in Food by Membrane Technology, pp 349-381, January 2019.

### Products and Compositions

The microorganisms and methods described herein can be used to produce a variety of products and compositions containing one or more HMOs. In some embodiments, a product suitable for animal consumption includes one or more HMO produced by the microorganisms or methods herein. The product can include one or more additional consumable ingredients, such as a protein, a lipid, a vitamin, a mineral or any combination thereof. The product can be suitable for mammalian consumption, human consumption or consumption as an animal feed or supplement for livestock and companion animals. In some embodiments, the product is suitable for mammalian consumption, such as for human consumption and is an infant formula, an infant food, a nutritional supplement or a prebiotic product. Products can have 1, 2, 3 or more than 3 HMOs, and one or more of the HMOs can be produced by the microorganisms or by the methods described herein. In some cases, the HMO is 3′-sialyllactose (3′-SL), 6′-sialyllactose (6′-SL), lacto-N-neotetraose (LNnT), lacto-N-tetraose (LNT), Lacto-N-Triose II (LNTII)-derived HMO or sialylated HMO or any combinations thereof. The HMO may be 3′-sialyllactose (3′-SL) or 6′-sialyllactose (6′-SL).

### EXAMPLES

### Example 1: LNT Production in Saccharomyces Cerevisiae Expressing a Heterologous Transporter

### Strains and Media

S. cerevisiae is grown and maintained on YPD medium (10 g/L yeast extract, 20 g/I, peptone, 20 g/L glucose) at 30° C. All genes are expressed chromosomally. The cdt-1sy gene and mutants are expressed within a background strain producing LNT and LNT accumulation in the growth medium during a fermentation experiment is compared to the LNT accumulation produced from the same strain with wild type cdt-1 gene.

The LNT producing S. cerevisiae strain contains genome integrated Lac12 and/or cdt-1 or a mutant thereof as transporter and LNT producing pathway consists of β1,3 GlcNAc Transferase (IgtA), β 1,3 Gal Transferase (wbgO).

Verduyn medium (See Verduyn et al., Yeast. 1992 July; 8(7):501-17) with 20 g/L of glucose (V20D) is used for preculture of yeast cells. Verduyn medium with 60 g/L glucose and 6 g/L lactose (V60D6L) is used for LNT production.

### Fermentation and Metabolite Analysis

Triplicates of single colonies are inoculated in 10 mL of Verduyn medium with 20 g/L glucose and incubated at 30° C. overnight. The cell cultures are centrifuged and resuspended in 10 mL V60D6L medium and incubated at 30° C. and 250 rpm for 48 hours. Extracellular lactose, glucose, and LNT concentration is determined by high performance liquid chromatography (HPLC) equipped with Rezex ROA-Organic Acid H 10×7.8 mm column and a refractive index detector (RID). The column is eluted with 0.005 N of sulfuric acid at a flow rate of 0.6 mL/min, 50° C. To measure total (intracellular and extracellular) LNT, the fermentation broth containing yeast cells is boiled to release all of the intracellular LNT. The supernatant is then analyzed by HPLC.

The extracellular and total LNT titer (in percentage) is normalized by the titer of strains with no transporter and/or with a wild type cdt-1 or lac12. Extracellular LNT ratio (%) is calculated as follows: (extracellular LNT titer)/(total LNT titer)×100%.

### Example 2: LNnT Production in Saccharomyces cerevisiae Expressing a Heterologous Transporter

### Strains and Media

S. cerevisiae was grown and maintained on YPD medium (10 g/L yeast extract, 20 g/L peptone, 20 g/L glucose) at 30° C. All transporter genes were expressed chromosomally, whereas pathway genes were expressed from plasmids. The cdt-1sy gene and mutants were expressed within a background strain producing LNnT, and LNnT accumulation in the growth medium and in the total cell culture samples during a fermentation experiment were compared to the LNnT accumulation produced from the same strain with a wild type cdt-1 gene and to a strain containing no transporter.

The LNnT producing S. cerevisiae strain contained genome integrated coat-1 or a mutant thereof as transporter and LNnT producing pathway consisting of β 1,3 GlcNAc Transferase (IgtA) and β 1,4 Gal Transferase (lgtB).

Verduyn medium (See Verduyn et al., Yeast. 1992 July; 8(7):501-17) with 20 g/L, of glucose (V20D) was used for preculture of yeast cells. Verduyn medium with 60 g/L glucose and 1 g/L lactose (V60D6L) was used for LNnT production.

### Fermentation and Metabolite Analysis

A single colony was inoculated in 10 mL of Verduyn medium with 20 g/L glucose and incubated at 30° C. overnight. The cell cultures were centrifuged and resuspended in 30 mL V60D1L medium and incubated at 30° C. and 250 rpm for 72 hours. Extracellular lactose and glucose concentrations were determined by high performance liquid chromatography (HPLC) equipped with Rezex ROA-Organic Acid H 10×7.8 mm column and a refractive index detector (RID). The column was eluted with 0.005 N of sulfuric acid at a flow rate of 0.6 mL/min, 50° C. To measure total (intracellular and extracellular) LNnT, the fermentation broth containing yeast cells was boiled to release all of the intracellular LNnT. The supernatant was then analyzed as described in Example 5; alternatively the LNnT can be analyzed by HPLC or Dionex.

The extracellular and total LNnT titer (shown in percentage) is normalized by the titer of strains with no transporter and/or with wild type cdt-1. Extracellular LNnT ratio (%) is calculated as follows: (extracellular LNnT titer)/(total LNnT titer)×100% Alternatively, samples were analyzed as shown in Example 5).

Lactose concentrations were measured from the shake flask experiments after 3 days of growth. Table 3 shows the residual lactose present, and demonstrates that the CDT-1 expressing strains import and utilize more lactose as compared to a no transporter control.

TABLE 3
Extracellular lactose measurements

| | Extracellular |
|---|---|
| Transporter | Lactose (g/L) |
| CDT-1 | 0.48 |
| CDT-1 G91A | 0.72 |
| CDT-1 F213L | 0.36 |
| CDT-1 L256V | 0.43 |
| CDT-1 F335A | 0.41 |
| CDT-1 S411A | 0.44 |
| CDT-1 N209S/F262W | 0.32 |
| CDT-1 N209S/F262Y first | 0.34 |
| 30 a.a. codon optimized | |
| Without CDT-1 | 1.00 |

### Example 3: 3′-SL Production in Saccharomyces cerevisiae Expressing a Heterologous Transporter

### Strains and Media

S. cerevisiae was grown and maintained on YPD medium (10 g/L yeast extract, 20 g/L peptone, 20 g/L glucose) at 30° C. All transporter genes were expressed chromosomally and pathway genes were expressed on plasmids. The cdt-Ty gene and mutants were expressed within a background strain producing 3′-SL, and 3′-SL accumulation in the growth medium during a fermentation experiment was compared to the 3′-SL accumulation produced from the same strain expressing the wild type cdt-1 gene and no transporter.

The 3′-SL producing strain contains genome integrated Lac12 and/or cdt-1 or a mutant thereof as transporter and the 3′-SL producing pathway consisted of GlcNAc 2-epimerase (nerd) (EC 5.1.3.8), NeuNAc Synthase (neuB) (EC 2.5.1.56), CMP-NeuNAc Synthetase (neuA) (EC:2.7.7.43), and α-2,3-sialyltransferase (EC 2.4.99.4) expressed episomally. Additionally, strains were created which omitted the pathway genes neuB and neuC genes.

Verduyn medium (See Verduyn et al., Yeast. 1992 July; 8(7):501-17) with 20 g/L of glucose (V20D) was used for preculture of yeast cells. Verduyn medium with 60 g/L glucose and 1 g/L lactose (V60D1L) and 0.25 g/L sialic acid was used for 3′-SL production for strains lacking neuB and neuC.

### Fermentation and Metabolite Analysis

A single colony was inoculated in 10 mL V20D and incubated at 30° C. overnight. The cell cultures were centrifuged and resuspended in 30 mL V60D1L medium with 0.25 g/L sialic acid and incubated at 30° C. and 250 rpm for 72 hours. Extracellular lactose, glucose concentration was determined by high performance liquid chromatography (HPLC) equipped with Rezex ROA-Organic Acid H 10×7.8 mm column and a refractive index detector (RID). The column was eluted with 0.005 N of sulfuric acid at a flow rate of 0.6 mL/min, 50° C. 3′-SL concentration may be determined using Dionex ICS-5000+ with a CarboPac PA-200 column; however the 3′-SL concentration in this study was determined as described in Example 5. The column is eluted with 100 mM sodium acetate (pH 4.0) containing 100 mM sodium hydroxide at a flow rate of 0.5 mL/min. The concentration of 3′-SL is calculated based on the peak area as compared to 3′-SL standards. To measure total (intracellular and extracellular) 3′-SL, the fermentation broth containing yeast cells was boiled to release all of the intracellular 3′-SL. The supernatant is then analyzed by Dionex ICS-5000+. Alternatively, 3′-SL abundance was determined as described in Example 5 using QQQ mass spectrometry.

The extracellular and total 3′-SL titer (shown in percentage) is normalized by the titer of strains with no transporter and/or with wild type cdt-1 or lac12. Extracellular 3′-SL ratio (%) is calculated as follows: (extracellular 3′-SL titer)/(total 3′-SL titer)×100%. Alternatively, results were analyzed as described in Example 5.

Lactose concentrations were measured from the shake flask experiments after 3 days of growth. Table 4 shows the residual lactose present, and demonstrates that the CDT-1 expressing strains import and utilize more lactose as compared to a no transporter control.

TABLE 4
Residual Lactose Measurements

| | Extracellular |
|---|---|
| Transporter | Lactose (g/L) |
| CDT-1 | 0.53 |
| CDT-1 G91A | 0.73 |
| CDT-1 F213L | 0.36 |
| CDT-1 L256V | 0.69 |
| CDT-1 F335A | 0.64 |
| CDT-1 S411A | 0.45 |
| CDT-1 N209S/F262W | 0.32 |
| CDT-1 N209S/F262Y first | 0.62 |
| 30 a. a. codon optimized | |
| Without CDT-1 | 0.91 |

### Example 4: 6′-SL Production in Saccharomyces cerevisiae Expressing a Heterologous Transporter

### Strains and Media

S. cerevisiae is grown and maintained on YPD medium (10 g/I. yeast extract, 20 g/L peptone, 20 g/L glucose) at 30° C. All genes are expressed chromosomally. The cdt-1sy gene and mutants are expressed within a background strain producing 6′-SL and 6′-SL accumulation in the growth medium during a fermentation experiment is compared to the 6′-SL accumulation produced from the same strain with wild type cdt-1 gene.

The 6′-SL producing strain contains genome integrated Lac12 and/or cdt-1 or a mutant thereof as transporter and 6′-SL producing pathway consists of GlcNAc 2-epimerase (neuC) (EC 5.1.3.8), NeuNAc Synthase (neuB) (EC 2.5.1.56), CMP-NeuNAc Synthetase (neuA) (EC:2.7.7.43), and α-2,6-sialyltransferase (EC 2.4.99.1).

Verduyn medium (See Verduyn et al., Yeast. 1992 July; 8(7):501-17) with 20 g/L of glucose (V20D) is used for preculture of yeast cells. Verduyn medium with 60 g/L glucose and 6 g/L lactose (V60D6L) is used for 6′-SL production.

### Fermentation and Metabolite Analysis

Triplicates of single colonies are inoculated in 10 mL V20D and incubated at 30° C. overnight. The cell cultures are centrifuged and resuspended in 10 mL V60D6L medium and incubated at 30° C. and 250 rpm for 48 hours. Extracellular lactose, glucose concentration is determined by high performance liquid chromatography (HPLC) equipped with Rezex ROA-Organic Acid H 10×7.8 mm column and a refractive index detector (RID). The column is eluted with 0.005 N of sulfuric acid at a flow rate of 0.6 mL/min, 50° C. 6′-SL concentration is determined using Dionex ICS-5000+ with a CarboPac PA-200 column. The column is eluted with 100 mM sodium acetate (pH 4.0) containing 100 mM sodium hydroxide at a flow rate of 0.5 mL/min. The contents of 6′-SL is calculated based on the peak area as compared to 6′-SL standards. To measure total (intracellular and extracellular) 6′-SL, the fermentation broth containing yeast cells is boiled to release all of the intracellular 6′-SL. The supernatant is then analyzed by Dionex ICS-5000+.

The extracellular and total 6′-SL titer (shown in percentage) is normalized by the titer of strains with no transporter and/or with wild type cdt-1 or lac12. Extracellular 6′-SL ratio (%) is calculated as follows: (extracellular 6′-SL titer)/(total 6′-SL titer)×100%.

### Example 5: Analysis of 3′-SL and LNnT from Yeast Cultures

### Oligosaccharide Extraction

Oligosaccharides were extracted from biological samples (extracellular and total) produced in Examples 2 and 3 following the procedure of Robinson et. al. with minor modification. Samples were centrifuged at 4,000×g for 10 min at room temperature to collect solids, and 250 μL aliquots of the supernatant were transferred to new tubes in duplicate. Two volumes of 500 μL cold ethanol were added to each aliquot and the samples were vortexed briefly before incubation for 1 hour at −30° C. The samples were centrifuged at 4,000×g for 30 min at 4° C. to collect precipitated proteins; the supernatant was subsequently dried by centrifugal evaporation (Genevac MiVac Quattro concentrator, Genevac Ltd., Ipswitch, England).

The samples were re-dissolved in 200 μL 18.2 MΩ·cm (Milli-Q) water and purified by microplate C18 solid phase extraction (Glygen, Columbia, Md., USA). The C18 microplates were conditioned with acetonitrile (ACN) and equilibrated with water. After sample loading the plate was washed with 600 μL of Milli-Q water. The eluate collected during and after sample loading was further purified by microplate graphitized carbon solid phase extraction (Glygen). The graphitized carbon microplates were conditioned with 80% ACN/0.1% trifluoroacetic acid (TFA) and equilibrated with 4% ACN/0.1% TFA. After sample loading the microplate was washed with 1.2 mL of 4% ACN/0.1% TFA. The oligosaccharides were eluted with 600 μL of 40% ACN/0.1% TFA and dried by centrifugal evaporation. The samples were re-dissolved in 400 μL Milli-Q water, diluted 5-fold, and spiked with appropriately diluted xylosyl cellobiose (Megazyme, Bray, Ireland) used as an internal standard for analysis by triple quadrupole mass spectrometry.

### Relative Oligosaccharide Quantification by Liquid Chromatography-Mass Spectrometry

The purified oligosaccharides were chromatographically separated with an Agilent 1260 Infinity II binary pump (Agilent Technologies, Santa Clara, Calif., USA) equipped with an AdvanceBio Glycan Mapping column (2.1×150 mm, 2.7 μm, Agilent Technologies) and an AdvanceBio Glycan Mapping guard column (2.1×5 mm, 2.7 μm, Agilent Technologies). The column temperature was maintained at 35° C. and 1.0 μL of each sample was injected in duplicate. Mobile phase solvents consisted of 3% ACN and 10 mM ammonium acetate in water (A) and 95% ACN with 10 mM ammonium acetate in water (B), each buffered to pH 4.5. The flow rate was set to 0.3 mL/min and the chromatographic gradient was programmed as follows: 0-4 min, 87% B; 4-S min, 87-80% B; 5-9 min, 80-72% B; 9-11 min, 72-57% B; 11-12 min, 57% B; 12-12.5 min, 57-87% B; 12.5-23 min, 87% B.

Following separation, the oligosaccharides were analyzed with an Agilent 6470A triple quadrupole (QQQ) mass spectrometer, equipped with a Jet Stream source (Agilent Technologies). The ionization source drying gas was operated at a flow of 10 L/min and temperature 150° C. Sheath gas flow and temperature were 7 L/min and 350° C., respectively; nebulizer pressure was 45 PSI; capillary voltage was 2200 V; and nozzle voltage was 0V. All data were collected in multiple reaction monitoring (MRM) mode and positive polarity. Two transitions were monitored for each analyte, as described in the Table 5. The default tolerance for each MRM qualifier or quantifier transition identification was set to a default of ±20%. Ion abundances in all samples were compared against a 1 mg/L standard for each respective analyte. Because Lacto-N-tetraose (LNT) did not chromatographically separate from its structural isomer of interest, LNnT, the combined ion abundance for both isomers were reported.

TABLE 5
Optimized MRM conditions for target oligosacchardes.

| | | | | | | Cell | |
|---|---|---|---|---|---|---|---|
| | | | | | Dwell | accelerator | Collision |
| | Precursor | Product | Quantifier/ | Fragmentor | time | voltage | energy |
| Compound | ion m/z | ion m/z | qualifier | (V) | (ms) | (V) | (V) |
| LNnT + | 708.3 | 366.0 | Quantifier | 115 | 25 | 2 | 13 |
| LNT | | | | | | | |
| LNnT + | 708.3 | 204.0 | Qualifier | 115 | 25 | 2 | 33 |
| LNT | | | | | | | |
| 3′-SL | 634.2 | 292.0 | Quantifier | 120 | 25 | 3 | 13 |
| 3′-SL | 634.2 | 274.0 | Qualifier | 120 | 25 | 3 | 25 |
| Xylosyl | 499.2 | 367.0 | Quantifier | 190 | 25 | 7 | 39 |
| cellobiose | | | | | | | |
| Xylosyl | 499.2 | 205.0 | Qualifier | 190 | 25 | 7 | 43 |
| cellobiose | | | | | | | |

### Data Analysis and Interpretation

Following injection and analysis, the raw data were processed in Agilent MassHunter Workstation Quantitative Analysis for QQQ, version 10.1. Chromatographic peaks were integrated and areas were exported in .csv format. Analyte quantitation was expressed as a ratio of either 3′-SL or LNnT/LNT ion abundance relative to the spiked 1 mg/L xylosyl cellobiose internal standard ion abundance. The limit of detection (LOD) for each analyte was defined as the average titer measured in a negative control strain lacking an exogenous CDT-1 (or mutant thereof) transporter gene (n=4) plus 3 standard deviations. The limit of quantitation (LOQ) for each analyte was defined as the average titer measured in a negative control strain lacking a CDT-1 transporter gene (n=4) plus 10 standard deviations. Oligosaccharide transport efficacy (n=2) was reported as a ratio of analyte abundance measured in the extracellular medium relative to its abundance measured in the total fraction. Ratios for transport efficacy were only reported if ion abundances were greater than or equal to the LOQ.

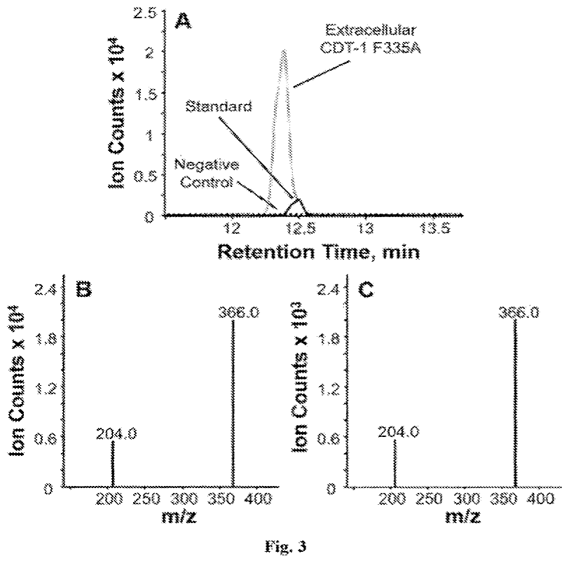

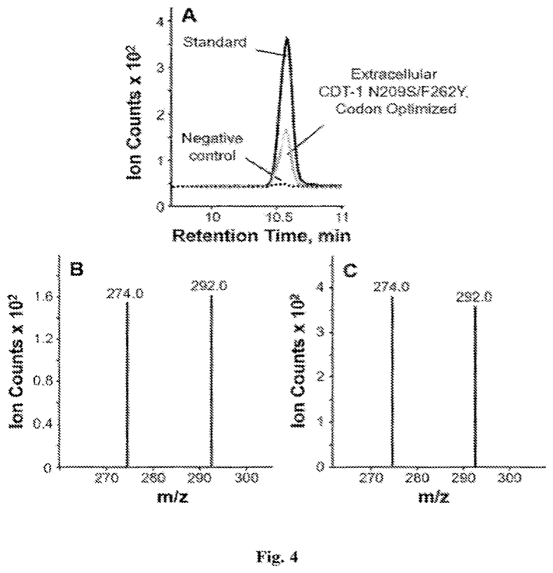

The data demonstrated that all strains produced the target oligosaccharide of interest and exported their respective product to the extracellular medium at quantifiable levels above a negative control strain lacking a product transporter (see FIGS. 3 and 4). For strains expressing the LNnT production pathway described in Example 2, strains harboring the CDT-1 L256V and CDT-1 N209S/F262W were most effective at product excretion to the extracellular medium, with the CDT-1 N209S/F262W and CDT-1 F335A mutants having the highest LNnT+LNT titers measured in the extracellular medium (Table 6). For strains expressing the 3′-SL pathway described in Example 3, 3′-SL was measured at >5-fold abundances in the extracellular medium of the CDT-1 N209S/F262Y codon optimized strain relative to wild type CDT-1; the extracellular/total ratio compared between CDT-1 mutants was within the range of reported assay measurement error.

TABLE 6
LNnT-related product abundance measured in S. cerevisiae mutant strains
harboring genes for LNnT overexpression and mutant CDT-1 transporters.

| | ^{a}Extracellular | ^{a}Total LNnT- | LNnT-related |
|---|---|---|---|
| | LNnT-related | related | product |
| | product/XC | product/XC | Extracellular/Total |
| Transporter | Ratio | Ratio | Ratio |
| CDT-1 | 8 (1) | 11 (1) | 0.7 (0.2) |
| CDT-1 G91A | 0.9 (0.2) | 1.2 (0.2) | 0.8 (0.2) |
| CDT-1 F213L | 2.7 (0.7) | 3.2 (0.9) | 0.9 (0.3) |
| CDT-1 L256V | 10 (1) | 8.3 (0.3) | 1.2 (0.1) |
| CDT-1 F335A | 14 (2) | 15 (3) | 0.9 (0.2) |
| CDT-1 S411A | 11 (3) | 13 (3) | 0.8 (0.3) |
| CDT-1 N209S/F262W | 15 (2) | 14 (3) | 1.0 (0.3) |
| CDT-1 N209S/F262Y first | 2.5 (0.7) | 3.1 (0.8) | 0.8 (0.3) |
| 30 a.a. codon optimized | | | |
| Without CDT-1 | ^{b}0.04 (0.01) | ^{b}0.04 (0.01) | ^{c}N.D. |

^{a}Defined as the proportion of total ion count abundance of LNnT-related product relative to the ion abundance of a spiked xylosyl cellobiose (XC) standard measured in each respective cellular fraction.
^{b}Negative control titers used to determine the limit of detection (LOD) and limit of quanititation (LOQ) for these measurements. LOD is defined as the average LNnT related product/XC ratio measured in the negative control plus 3 standard deviations; LOQ is defined as the same ratio measured in the negative control plus 10 standard deviations.
^{c}Denotes Not Determined. LNnT-related product denotes the abundance of LNnT, and may contain some amounts of LNT, which was not distinguishable under these conditions.

TABLE 7
3′-SL abundances measured in S. cerevisiae mutant strains harboring
genes for 3′-SL overexpression and mutant CDT-1 transporters

| | | | 3-SL |
|---|---|---|---|
| | ^{a}Extracellular | ^{a}Total 3′- | Extracellular/ |
| | 3′-SL/XC | SL/XC | Total |
| Transporter | Ratio × 10² | Ratio × 10² | Ratio |
| CDT-1 | 1.2 (0.3) | 0.8 (0.2) | 1.5 (0.6) |
| CDT-1 G91A | 1.6 (0.2) | 1.1 (0.2) | 1.5 (0.4) |
| CDT-1 F213L | 2.4 (0.7) | 1.4 (0.2) | 1.8 (0.6) |
| CDT-1 L256V | 2.2 (0.3) | 1.8 (0.2) | 1.2 (0.2) |
| CDT-1 F335A | 2.8 (0.5) | 1.3 (0.4) | 2.2 (0.8) |
| CDT-1 S411A | 1.0 (0.2) | ^{c}0.3 (0.1) | ^{d}N.D. |
| CDT-1 N209S/F262W | 0.8 (0.2) | ^{c}0.3 (0.04) | ^{d}N.D. |
| CDT-1 N209S/F262Y | 6.0 (0.2) | 3.6 (0.7) | 1.8 (0.7) |
| first 30 a.a. codon optimized | | | |
| Without CDT-1 | ^{b}0.4 (0.1) | ^{b}0.4 (0.1) | ^{d}N.D. |

^{a}Defined as the proportion of total ion count abundance of 3′-SL relative to the ion abundance of a spiked xylosyl cellobiose (XC) standard measured in each respective cellular fraction.
^{b}Negative control titers used to determine the limit of detection (LOD) and limit of quanititation (LOQ) for these measurements. LOD is defined as the average 3′-SL/XC ratio measured in the negative control plus 3 standard deviations; LOQ is defined as the same ratio measured in the negative control plus 10 standard deviations.
^{c}Measurements falling below the limit of detection cutoff.
^{d}Denotes Not Determined.

### REFERENCE FOR DETECTION METHODS

Robinson, R. C., Poulsen, N. A., Colet, E., Duchene, C., Larsen, L. B., Barile, D. Profiling of aminoxy TMT-labeled bovine milk oligosaccharides reveals substantial variation in oligosaccharide abundance between dairy cattle breeds. Scientific Reports. 2019, 9, 5465.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments described herein. Such equivalents are intended to be encompassed by the following claims.

We claim:

1. An engineered microorganism capable of producing a human milk oligosaccharide (HMO) comprising:
a first heterologous gene encoding an HMO formation enzyme and a second heterologous gene encoding a transporter for export of the HMO, wherein the transporter is CDT-1 or a variant thereof,
wherein the HMO is a Lacto-N-Triose II (LNTII)-derived HMO or a sialylated HMO.

2. The engineered microorganism of claim 1, wherein the HMO is a LNTII-derived HMO selected from lacto-N-neotetraose (LNnT) or lacto-N-tetraose (LNT).

3. The engineered microorganism of claim 1, wherein the HMO is a sialylated HMO selected from 3′-sialyllactose (3′-SL) or 6′-sialyllactose (6′-SL).

4. The engineered microorganism according to any of claims 1-3, wherein the transporter is a variant of CDT-1.

5. The microorganism according to any one of claims 1-4, wherein the CDT-1 or variant thereof has an amino acid sequence of SEQ ID NO: 4 or a sequence with at least 80%, 85%, 90%, 95%, 98% or 99% homology thereto.

6. The microorganism according to any one of claims 1-5, wherein the CDT-1 or variant thereof comprises a PESPR motif (SEQ ID NO: 43).

7. The microorganism according to any one of claims 1-6, wherein the CDT-1 variant comprises a sequence having one or more amino acid replacements at positions corresponding to amino acid positions 91, 209, 213, 256, 262, 335, 411 of SEQ ID NO:4.

8. The microorganism according to any one of claims 1-7, wherein the CDT-1 or variant thereof is encoded by a codon optimized nucleic acid.

9. The microorganism according to claim 8, wherein at least the first 90 nucleotides of the nucleic acid are codon optimized for yeast or at least 5% of the nucleic acid is codon optimized for yeast.

10. The microorganism according to any one of claims 7-9, wherein the CDT-1 variant comprises an amino acid replacement selected from the group consisting of 91A, 209S, 213L, 256V, 262Y, 262W, 335A, 411A and any combination thereof.

11. The engineered microorganism according to any one of claims 1-10, wherein the CDT-1 variant is selected from the group consisting of CDT-1 N209S F262Y, CDT-1 G91A, CDT-1 F213L, CDT-1 L256V, CDT-1 F335A, CDT-1 S411A, and CDT-1 N209S F262W, or wherein the CDT-1 variant comprises an amino acid replacement at a position near the sugar substrate binding pocket and/or the PESPR motif (SEQ ID NO: 43), such as G336, Q337, N341, or G471.

12. The engineered microorganism according to any of claim 1-12, wherein the engineered microorganism utilizes lactose as an HMO substrate.

13. The engineered microorganism according to any of claim 4-12, wherein the variant of CDT-1 is capable of lactose import and HMO export.

14. The engineered microorganism of any one of claims 4-13, wherein the variant of CDT-1 has an increased capability of lactose import as compared to CDT-1 (SEQ ID NO: 4).

15. The engineered microorganism of any one of claims 4-13, wherein the variant of CDT-1 has an increased capability of HMO export as compared to CDT-1 (SEQ ID NO: 4).

16. The engineered microorganism according to any one of claims 1-15, wherein the engineered microorganism further comprises a genetic modification encoding a second transporter for import of an HMO substrate.

17. The engineered microorganism of claim 16, wherein the second transporter is lac12 or a variant thereof.

18. The engineered microorganism of claim 17, wherein the lac12 has an amino acid sequence of SEQ ID NO: 41 or a sequence with at least 80%, 85%, 90%, 95%, 98% or 99% homology thereto.

19. The engineered microorganism according to any one of claims 1-18, wherein the microorganism is selected from the group consisting of an Ascomycetes fungus, a Saccharomyces spp., a Schizosaccharomyces spp., a Pichia spp., Trichoderma, Kluyveromyces, Yarrowia, Aspergillus, and Neurospora.

20. The engineered microorganism according to any one of claims 1-19, wherein the HMO formation enzyme is a β 1,3 GlcNAc Transferase or a glycosyltransferase.

21. The engineered microorganism of claim 20, wherein the β 1,3 GlcNAc Transferase is encoded by lgtA.

22. The microorganism of claim 20 or claim 21, wherein the β 1,3 GlcNAc Transferase has an amino acid sequence selected from SEQ ID NOs: 17-19, 42, or a sequence with at least 80%, 85%, 90%, 95%, 98% or 99% homology thereto.

23. The engineered microorganism according to any one of claims 1-19, wherein the HMO formation enzyme is a β 1,3 Gal Transferase.

24. The engineered microorganism of claim 23, wherein the β 1,3 Gal Transferase is encoded by wbgO.

25. The microorganism of claim 23 or claim 24, wherein the β 1,3 Gal Transferase has an amino acid sequence selected from SEQ ID NOs: 20-22 or a sequence with at least 80%, 85%, 90%, 95%, 98% or 99% homology thereto.

26. The engineered microorganism according to any one of claims 1-19, wherein the HMO formation enzyme is a β 1,4 Gal Transferase.

27. The engineered microorganism of claim 26, wherein the β 1,4 Gal Transferase is encoded by lgtB.

28. The microorganism of claim 26 or claim 27, wherein the β 1,4 Gal Transferase has an amino acid sequence selected from SEQ ID NOs: 23-25 or a sequence with at least 80%, 85%, 90%, 95%, 98% or 99% homology thereto.

29. The engineered microorganism according to any one of claims 1-19, wherein the HMO formation enzyme is a NeuNAc Synthase.

30. The microorganism of claim 29, wherein the NeuNAc Synthase has an amino acid sequence selected from SEQ ID NOs: 26-28 or a sequence with at least 80%, 85%, 90%, 95%, 98% or 99% homology.

31. The engineered microorganism according to any one of claims 1-19, wherein the HMO formation enzyme is a α-2,6-sialyltransferase.

32. The microorganism of claim 31, wherein the α-2,6-sialyltransferase has an amino acid sequence of SEQ ID NO: 34 or a sequence with at least 80%, 85%, 90%, 95%, 98% or 99% homology.

33. The engineered microorganism according to any one of claims 1-19, wherein the HMO formation enzyme is a CMP-NeuNAc Synthetase.

34. The microorganism of claim 33, wherein the CMP-NeuNAc Synthetase has an amino acid sequence selected from SEQ ID NOs: 29-30 or a sequence with at least 80%, 85%, 90%, 95%, 98% or 99% homology.

35. The engineered microorganism according to any one of claims 1-19, wherein the HMO formation enzyme is a α-2,3-sialyltransferase.

36. The microorganism of claim 35, wherein the α-2,3-sialyltransferase has an amino acid sequence selected from SEQ ID NOs: 31-33 or a sequence with at least 80%, 85%, 90%, 95%, 98% or 99% homology.

37. The engineered microorganism according to any one of claims 1-19, wherein the HMO formation enzyme is a sialyltransferase (PmST).

38. The microorganism of claim 37, wherein the sialyltransferase (PmST) has an amino acid sequence of SEQ ID NO: 35 or a sequence with at least 80%, 85%, 90%, 95%, 98% or 99% homology.

39. The engineered microorganism according to any one of claims 1-19, wherein the HMO formation enzyme is a UDP-GlcNAc 2-epimerase.

40. The microorganism of claim 39, wherein the UDP-GlcNAc 2-epimerase has an amino acid sequence selected from SEQ ID NOs: 36-40 or a sequence with at least 80%, 85%, 90%, 95%, 98% or 99% homology.

41. The engineered microorganism according to any one of claims 1-19, wherein the HMO is a sialylated and the HMO formation enzyme is selected from the group consisting of slr1975 gene from Synechocystis sp. PCC6803, nanA gene from E. coli W3110, slr1975 gene from Synechocystis sp. PCC6803, neuB gene from E. coli K1, age from Anabaena sp. CH1, neuB from E. coli K12, α-2,3-sialyltransferase gene from Neisseria gonorrhoeae, α-2,6-sialyltransferase from Photobacterium sp. JT-ISH-224, neuC from Campylobacter jejuni, neuB from C. jejuni ATCC 43438, neuA from C. jejuni ATCC 43438, sialyltransferase PmST from Pasteurella multocida, neuB from N. meningitidis MC58 group B, neuC gene from N. meningitidis MC58 group B, Sialidase (Tr6) from Trypanosoma rangeli, alpha-2,3-sialyltransferase from Neisseria meningitidis, NeuNAc Synthase from Campylobacter jejuni, and CMP-NeuNAc Synthetase from Neisseria meningitidis.

42. The engineered microorganism of claim 41, wherein the microorganism comprises CMP-NeuNAc Synthetase and α-2,3-sialyltransferase, and wherein the engineered microorganism is capable of producing a sialylated HMO when grown in the presence of sialic acid.

43. The microorganism according to any one of claims 1-42, wherein the gene encoding the transporter and the gene encoding the formation enzyme are integrated into the microorganism chromosome.

44. The microorganism according to any one of claims 1-42, wherein the gene encoding the transporter and the gene encoding the formation enzyme are episomal.

45. The microorganism according to any one of claims 1-44, wherein the microorganism is capable of producing and exporting the HMO.

46. The microorganism according to any one of claims 1-45, wherein CUT-1 is capable of exporting at least 20%, 30%, 40%, 50%, or 60% of the produced LIMO.

47. The microorganism according to any one of claims 1-45, wherein the microorganism is capable of exporting at least 50% more of the HMO than a parental microorganism lacking the transporter.

48. A method of producing an HMO comprising:
providing the engineered microorganism according to any of claims 1-47, wherein the engineered microorganism is capable of producing and exporting an HMO; and
culturing the engineered microorganism in the presence of a substrate;
wherein a substantial portion of the HMO is exported into the culture medium.

49. The method of claim 48, further comprising separating the culture medium from the engineered microorganism.

50. The method of claim 48 or claim 49, further comprising isolating the HMO from the culture medium.

51. The method according to any of claims 48-50, wherein the substrate is selected from the group consisting of lactose, UDP-galactose, Pyruvate/PEP, and CTP.

52. The method of claim 51, wherein the microorganism is cultured in the presence of sialic acid.

53. The method according to any one of claims 48-52, wherein the transporter is capable of importing lactose and/or exporting the HMO.

54. The method according to any one of claims 48-53, wherein the culture medium comprises lactose.

55. A product suitable for animal consumption comprising the microorganism according to any one of claims 1-47 and an HMO produced by the engineered microorganism according to any one of claims 1-47.

56. A product suitable for animal consumption comprising the microorganism according to any one of claims 1-47 and the HMO produced according to the method of any one of claims 48-54.

57. The product of claim 55 or 56 further comprising at least one additional consumable ingredient.

58. The product of claim 57, wherein the additional consumable ingredient is selected from a protein, a lipid, a vitamin, a mineral or any combination thereof.

59. The product according to any of claims 55-58, wherein the product is suitable for human consumption.

60. The product of claim 59, wherein the product is an infant formula, an infant food, a nutritional supplement or a prebiotic product.

61. The product according to any of claims 55-58, wherein the product is suitable for mammalian consumption.

62. The product according to any of claims 55-58, wherein the product is suitable for use as an animal feed.

63. The product according to any of claims 55-62, further comprising at least one additional human milk oligosaccharide.

64. An engineered microorganism capable of producing a human milk oligosaccharide (HMO) comprising:
a first heterologous gene encoding an HMO formation enzyme and a second heterologous gene encoding a variant of CDT-1, wherein the CDT-1 variant comprises a sequence having one or more amino acid replacements at positions corresponding to amino acid positions 91, 209, 213, 256, 262, 335, 411 of SEQ ID NO:4, or the CDT-1 variant is selected from the group consisting of CDT-1 N209S F262Y, CDT-1 G91A, CDT-1 F213L, CDT-1 L256V, CDT-1 F335A, CDT-1 S411A, and CDT-1 N209S F262W, or the CDT-1 variant comprises an amino acid replacement at a position near the sugar substrate binding pocket and/or the PESPR motif (SEQ ID NO: 43), such as G336, Q337, N341, or G471; and
wherein the engineered microorganism produces an HMO and is improved in the uptake of lactose into the microorganism as compared to a parent microorganism that lacks CDT-1, or a variant thereof.

65. The method of claim 64, wherein the CDT-1 variant comprises a sequence having one or more amino acid replacements at positions corresponding to amino acid positions 91, 209, 256, 262, 335, 411 of SEQ ID NO:4.

66. The method of claim 64, wherein the CDT-1 variant is selected from the group consisting of CDT-1 N209S F262Y, CDT-1 G91A, CDT-1 L256V, CDT-1 F335A, CDT-1 S411A, and CDT-1 N209S F262W.

67. The method of any of claims 64-66, wherein the HMO is a Lacto-N-Triose II (LNTII)-derived HMO or a sialylated HMO.

68. The method of any of claims 64-66, wherein the HMO is selected from the group consisting of lacto-N-neotetraose (LNnT), lacto-N-tetraose (LNT), 3′-sialyllactose (3′-SL) and 6′-sialyllactose (6′-SL).
